(19) United States
(12) Patent Application Publication
(10) Pub. No.: US 2025/0146000 A1
(43) Pub. Date: May 8, 2025

(54) COMPOSITIONS AND METHODS OF TREATING AMYOTROPHIC LATERAL SCLEROSIS (ALS)

(71) Applicant: Voyager Therapeutics, Inc., Lexington, MA (US)

(72) Inventors: Dinah Wen-Yee SAH, Hopkinton, MA (US); Jinzhao Hou, Lexington, MA (US); Mathieu E. Nonnenmacher, Cambridge, MA (US); Pengcheng Zhou, Lexington, MA (US); Markus Hossbach, Kulmbach (DE); Jochen Deckert, Bayreuth (DE)

(21) Appl. No.: 18/884,711

(22) Filed: Sep. 13, 2024

Related U.S. Application Data
(63) Continuation of application No. 18/085,222, filed on Dec. 20, 2022, now Pat. No. 12,123,002.
Application No. 18/085,222 is a continuation of application No. 17/143,036, filed on Jan. 6, 2021, now Pat. No. 11,542,506.
Application No. 17/143,036 is a division of application No. 16/774,493, filed on Jan. 28, 2020, now Pat. No. 10,920,227.
Application No. 16/774,493 is a division of application No. 15/526,690, filed on May 12, 2017, now Pat. No. 10,597,660.
(60) Provisional application No. 62/079,588, filed on Nov. 14, 2014.
(60) Provisional application No. 62/211,992, filed on Aug. 31, 2015.
(60) Provisional application No. 62/234,466, filed on Sep. 29, 2015.

(51) Int. Cl. C12N 15/113

(52) U.S. Cl. CPC C12N 15/1137; C12Y 115/01001; C12N 2310/14; C12N 2330/51; C12N 2750/14143

(57) ABSTRACT

The present invention relates to small interfering RNA (siRNA) molecules against the SOD1 gene, adeno-associated viral (AAV) vectors encoding siRNA molecules and methods for treating amyotrophic lateral sclerosis (ALS) using the siRNA molecules and AAV vectors.

### CROSS REFERENCE TO RELATED APPLICATIONS

This application is a continuation of U.S. patent application Ser. No. 18/085,222 (now issued as U.S. Pat. No. 12,123,002), filed on Dec. 20, 2022, which is a continuation of U.S. patent application Ser. No. 17/143,036 (now issued as U.S. Pat. No. 11,542,506), filed on Jan. 6, 2021, which is a divisional of U.S. patent application Ser. No. 16/774,493 (now issued as U.S. Pat. No. 10,920,227), filed Jan. 28, 2020, which is a divisional of U.S. patent application Ser. No. 15/526,690, (now issued as U.S. Pat. No. 10,597,660), filed May 12, 2017, which is a national stage filing under 35 U.S.C. § 371 of International Application No. PCT/US2015/060562, filed Nov. 13, 2015, which claims priority to U.S. Provisional Patent Application No. 62/079,588, filed Nov. 14, 2014, U.S. Provisional Patent Application No. 62/211,992, filed Aug. 31, 2015, and U.S. Provisional Patent Application No. 62/234,466, filed Sep. 29, 2015; the contents of each of which are herein incorporated by reference in their entirety.

### SEQUENCE LISTING

The instant application contains a Substitute Sequence Listing which has been submitted electronically in XML format and is hereby incorporated by reference in its entirety. Said XML copy, created on Nov. 21, 2024, is named VTJ-10¹¹USDV2CN.xml and is 664 kilobytes in size.

### FIELD OF THE INVENTION

The present invention relates to compositions, methods and processes for the design, preparation, manufacture, use and/or formulation of modulatory polynucleotides, e.g., small interfering RNA (siRNA) molecules which target the superoxide dismutase 1 (SOD1) gene. As used herein, a “modulatory polynucleotide” is any nucleic acid sequence(s) which functions to modulate (either increase or decrease) the level or amount of a target gene, e.g., mRNA or protein levels. Targeting of the SOD1 gene may interfere with SOD1 gene expression and SOD1 enzyme production. In some embodiments, the nucleic acid sequence encoding the siRNA molecule are inserted into recombinant adeno-associated virus (AAV) vectors. Methods for using the siRNA molecules to inhibit SOD1 gene expression in a subject with a neurodegenerative disease (e.g., amyotrophic lateral sclerosis (ALS)) are also disclosed.

### BACKGROUND OF THE INVENTION

Amyotrophic lateral sclerosis (ALS), also known as Lou Gehrig's disease, is the most fatal progressive neurodegenerative disease, characterized by the predominant loss of motor neurons (MNs) in primary motor cortex, the brainstem, and the spinal cord. The loss of motor neurons devastates basic, fundamental movements, such as breathing, and typically causes death to patients within 2˜5 years after diagnosis. Progressive deterioration of motor function in patients severely disrupts their breathing ability, requiring some form of breathing aid for survival of the patients. Other symptoms also include muscle weakness in hands, arms, legs or the muscles of swallowing. Some patients (e.g., FTD-ALS) may also develop frontotemporal dementia.

According to the ALS Association, approximately 5,600 people in the United States of America are diagnosed with ALS each year. The incidence of ALS is two per 100,000 people, and it is estimated that as many as 30,000 Americans may have the disease at any given time.

Two forms of ALS have been described: one is sporadic ALS (sALS), which is the most common form of ALS in the United States of America and accounts for 90 to 95% of all cases diagnosed; the other is familial ALS (fALS), which occurs in a family lineage mainly with a dominant inheritance and only accounts for about 5 to 10% of all cases in the United States of America. sALS and fALS are clinically indistinguishable.

Pathological studies found that disturbance of some cellular processes occur after disease onset, including increased ER stress, generation of free radicals (i.e., reactive oxygen species (ROS)), mitochondrial dysfunction, protein aggregation, apoptosis, inflammation, and glutamate excitotoxicity, specifically in the motor neurons (MNs).

The causes of ALS are complicated and heterogeneous. In general, ALS is considered to be a complex genetic disorder in which multiple genes in combination with environmental exposures combine to render a person susceptible. More than a dozen genes associated with ALS have been discovered, including, SOD-1 (Cu²⁺/Zn²⁺ superoxide dismutase), TDP-43 (TARDBP, TAR DNA binding protein-43), FUS (Fused in Sarcoma/Translocated in Sarcoma), ANG (Angiogenin), ATXN2 (Ataxin-2), valosin containing protein (VCP), OPTN (Optineurin) and an expansion of the noncoding GGGGCC hexanucleotide repeat in the chromosome 9, open reading frame 72 (C9ORF72). However, the exact mechanisms of motor neuron degeneration are still elusive.

Currently, there is no curative treatment for ALS. The only FDA approved drug is Riluzole, which antagonizes the glutamate response to reduce the pathological development of ALS. However, only about a three-month life span expansion for ALS patients in the early stages has been reported, and no therapeutic benefit for ALS patients in the late stages has been observed, indicating a lack of therapeutic options for the patients (Bensimon G et al., J Neurol. 2002, 249, 609-615). Therefore, a new treatment strategy that can effectively prevent the disease progression is still in demand.

Many different strategies are under investigation for potential treatment of both sporadic and familial ALS. One strategy is based on the neuroprotective and/or regenerative effect of neurotrophic factors, such as Insulin-like growth factor I (IGF-I), Glial cell line-derived neurotrophic factor (GDNF), Vascular endothelial growth factor (VEGF), Colivelin and Activity dependent neurotrophic factor (ADNF) derived peptide, which can promote neuronal survival. Several studies demonstrated that neurotrophic factors can preserve motor neuron functionality, therefore improving motor performance in the SOD1 transgenic mice. However, such treatment often fails to prolong the survival of SOD1 mice, suggesting that neurotrophic factors are not sufficient to prolong neuronal survival (See a review by Yacila and Sari, Curr Med Chem., 2014, 21 (31), 3583-3593).

Another strategy for ALS treatment has focused on stem cell-based therapy. Stem cells have the potential to generate motor neurons, thereby replacing degenerating motor neurons in the ALS-affected CNS such as primary motor cortex, brainstem, and spinal cord. Stem cells derived from multiple sources have been investigated, including induced pluripotent stem cells (iPSCs), mesenchymal stromal cells (MSCs) (e.g., bone marrow mesenchymal stromal cells (BMSCs) and adipocyte stem cells (ASCs)) and neural tissue origin neural stem cells (e.g., fetal spinal neural stem cells (NSCs), multipotent neural progenitor cells (NPCs)) (e.g., reviewed by Kim C et al., Exp. Neurobiol., 2014, 23 (3), 207-214).

Mutations in the gene of superoxide dismutase type I (SOD1; Cu²⁺/Zn²⁺ superoxide dismutase type I) are the most common cause of fALS, accounting for about 20 to 30% of all fALS cases. Recent reports indicate that SOD1 mutations may also be linked to about 4% of all sALS cases (Robberecht and Philip, Nat. Rev. Neurosci., 2013, 14, 248-264). SOD1-linked fALS is most likely not caused by loss of the normal SOD1 activity, but rather by a gain of a toxic function. One of the hypotheses for mutant SOD1-linked fALS toxicity proposes that an aberrant SOD1enzyme causes small molecules such as peroxynitrite or hydrogen peroxide to produce damaging free radicals. Other hypotheses for mutant SOD1 neurotoxicity include inhibition of the proteasome activity, mitochondrial damage, disruption of RNA processing and formation of intracellular aggregates. Abnormal accumulation of mutant SOD1variants and/or wild-type SOD1 in ALS forms insoluble fibrillar aggregates which are identified as pathological inclusions. Aggregated SOD1 protein can induce mitochondria stress (Vehvilainen P et al., Front Cell Neurosci., 2014, 8, 126) and other toxicity to cells, particularly to motor neurons.

These findings indicate that SOD1 can be a potential therapeutic target for both familial and sporadic ALS. A therapy that can reduce the SOD1 protein produced in the central nervous system of ALS patients may ameliorate the symptoms of ALS in patients such as motor neuron degeneration and muscle weakness and atrophy. Agents and methods that aim to prevent the formation of wild type and/or mutant SOD1 protein aggregation may prevent disease progression and allow for amelioration of ALS symptoms. RNA interfering (RNAi) mediated gene silencing has drawn researchers' interest in recent years. Small double stranded RNA (small interfering RNA) molecules that target the SOD1 gene haven been taught in the art for their potential in treating ALS (See, e.g., U.S. Pat. No. 7,632,938 and U.S. Patent Publication No. 20060229268, the contents of which is herein incorporated by reference in its entirety).

The present invention develops an RNA interference-based approach to inhibit or prevent the expression of SOD1 in ALS patients for treatment of the disease.

The present invention provides novel double stranded RNA (dsRNA) constructs and siRNA constructs and methods of their design. In addition, these novel siRNA constructs may be synthetic molecules or be encoded in an expression vector (one or both strands) for delivery into cells. Such vectors include, but are not limited to adeno-associated viral vectors such as vector genomes of any of the AAV serotypes or other viral delivery vehicles such as lentivirus, etc.

### SUMMARY OF THE INVENTION

The present invention relates to RNA molecule mediated gene specific interference with gene expression and protein production. Methods for treating motor neuron degeneration diseases such as amyotrophic lateral sclerosis are also included in the present invention. The siRNA included in the compositions featured herein encompass a dsRNA having an antisense strand (the antisense strand) having a region that is 30 nucleotides or less, generally 19-24 nucleotides in length, that is substantially complementary to at least part of an mRNA transcript of the SOD1 gene.

The present invention provides short double stranded RNA molecules such as small interfering RNA (siRNA) duplexes that target SOD1 mRNA to interfere with SOD1 gene expression and/or SOD1 protein production. The siRNA duplexes of the present invention may interfere with both alleles of the SOD1 gene irrespective of any particular mutation in the SOD1 gene, and may particularly interact with those found in ALS disease.

In some embodiments, such siRNA molecules, or a single strand of the siRNA molecules, are inserted into adeno-associated viral vectors to be introduced into cells, specifically motor neurons and/or other surrounding cells in the central nervous system.

The siRNA duplex of the present invention comprises an antisense strand and a sense strand hybridized together forming a duplex structure, wherein the antisense strand is complementary to the nucleic acid sequence of the targeted SOD1 gene, and wherein the sense strand is homologous to the nucleic acid sequence of the targeted SOD1 gene. In some aspects, the 5′end of the antisense strand has a 5′ phosphate group and the 3′end of the sense strand contains a 3′hydroxyl group. In other aspects, there are none, one or 2 nucleotides overhangs at the 3′end of each strand.

According to the present invention, each strand of the siRNA duplex targeting the SOD1 gene is about 19-25 nucleotides in length, preferably about 19 nucleotides, 20 nucleotides, 21 nucleotides, 22 nucleotides, 23 nucleotides, 24 nucleotides, or 25 nucleotides in length. In some aspects, the siRNAs may be unmodified RNA molecules.

In other aspects, the siRNAs may contain at least one modified nucleotide, such as base, sugar or backbone modification.

In one embodiment, an siRNA or dsRNA includes at least two sequences that are complementary to each other. The dsRNA includes a sense strand having a first sequence and an antisense strand having a second sequence. The antisense strand includes a nucleotide sequence that is substantially complementary to at least part of an mRNA encoding SOD1, and the region of complementarity is 30 nucleotides or less, and at least 15 nucleotides in length. Generally, the dsRNA is 19 to 24, e.g., 19 to 21 nucleotides in length. In some embodiments the dsRNA is from about 15 to about 25 nucleotides in length, and in other embodiments the dsRNA is from about 25 to about 30 nucleotides in length.

The dsRNA, either upon contacting with a cell expressing SOD1 or upon transcription within a cell expressing SOD1, inhibits or suppresses the expression of a SOD1 gene by at least 10%, at least 20%, at least 25%, at least 30%, at least 35% or at least 40% or more, such as when assayed by a method as described herein.

According to the present invention, AAV vectors comprising the nucleic acids encoding the siRNA duplexes, one strand of the siRNA duplex or the dsRNA targeting SOD1 gene are produced, the AAV vector serotype may be AAV1, AAV2, AAV3, AAV4, AAV5, AAV6, AAV7, AAV8, AAV9, AAV9.47, AAV9 (hu14), AAV10, AAV11, AAV12, AAVrh8, AAVrh10, AAV-DJ8 and/or AAV-DJ, and variants thereof.

According to the present invention, siRNA duplexes or dsRNA targeting the SOD1 gene in ALS are selected from the siRNA duplexes listed in Table 3, 11 or 13. Preferably, the siRNA duplexes or dsRNA targeting SOD1 gene in ALS are selected from the group consisting of siRNA duplexes: D-2757, D-2806, D-2860, D-2861, D-2875, D-2871, D-2758, D-2759, D-2866, D-2870, D-2823 and D-2858.

The present invention also provides pharmaceutical compositions comprising at least one siRNA duplex targeting the SOD1 gene and a pharmaceutically acceptable carrier. In some aspects, a nucleic acid sequence encodingthe siRNA duplex is inserted into an AAV vector.

In some embodiments, the present invention provides methods for inhibiting/silencing SOD1 gene expression in a cell. Accordingly, the siRNA duplexes or dsRNA can be used to substantially inhibit SOD1 gene expression in a cell, in particular in a motor neuron. In some aspects, the inhibition of SOD1 gene expression refers to an inhibition by at least about 20%, preferably by at least about 30%, 40%, 50%, 60%, 70%, 80%, 85%, 90%, 95% and 100%. Accordingly, the protein product of the targeted gene may be inhibited by at least about 20%, preferably by at least about 30%, 40%, 50%, 60%, 70%, 80%, 85%, 90%, 95% and 100%. The SOD1 gene can be either a wild type gene or a mutated SOD1 gene with at least one mutation. Accordingly, the SOD1 protein is either wild type protein or a mutated polypeptide with at least one mutation.

In some embodiments, the present invention provides methods for treating, or ameliorating amyotrophic lateral sclerosis associated with abnormal SOD1 gene and/or SOD1 protein in a subject in need of treatment, the method comprising administering to the subject a pharmaceutically effective amount of at least one siRNA duplex targeting the SOD1 gene, delivering said siRNA duplex into targeted cells, inhibiting SOD1 gene expression and protein production, and ameliorating symptoms of ALS in the subject.

In some embodiments, an AAV vector comprising the nucleic acid sequence encoding at least one siRNA duplex targeting the SOD1 gene is administered to the subject in need for treating and/or ameliorating ALS. The AAV vector serotype may be selected from the group consisting of AAV1, AAV2, AAV3, AAV4, AAV5, AAV6, AAV7, AAV8, AAV9, AAV9.47, AAV9 (hu14), AAV10, AAV11, AAV12, AAVrh8, AAVrh10 and AAV-DJ, and variants thereof.

In some aspects, ALS is familial ALS linked to SOD1 mutations. In other aspects, ALS is sporadic ALS which is characterized by abnormal aggregation of SOD1 protein or disruption of SOD1 protein function or localization, though not necessarily as a result of genetic mutation. The symptoms of ALS ameliorated by the present method may include motor neuron degeneration, muscle weakness, stiffness of muscles, slurred speech and/or difficulty in breathing.

In some embodiments, the siRNA duplexes or dsRNA targeting SOD1 gene or the AAV vectors comprising such siRNA-encoding molecules may be introduced directly into the central nervous system of the subject, for example, by intracranial injection.

In some embodiments, the pharmaceutical composition of the present invention is used as a solo therapy. In other embodiments, the pharmaceutical composition of the present invention is used in combination therapy. The combination therapy may be in combination with one or more neuroprotective agents such as small molecule compounds, growth factors and hormones which have been tested for their neuroprotective effect on motor neuron degeneration.

In some embodiments, the present invention provides methods for treating, or ameliorating amyotrophic lateral sclerosis by administering to a subject in need thereof a therapeutically effective amount of a plasmid or AAV vector described herein. The ALS may be familial ALS or sporadic ALS.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead being placed upon illustrating the principles of various embodiments of the invention.

FIG. 1 is a histogram showing the activity of the constructs encoded in an AAV vector.

FIG. 2 is a histogram showing the activity of the guide strand of the modulatory polynucleotides encoded in an AAV vector in HEK293T cells.

FIG. 3 is a histogram showing the activity of the passenger strand of the modulatory polynucleotides encoded in an AAV vector in HEK293T cells.

FIG. 4 is a histogram showing the activity of the guide strand of the modulatory polynucleotides encoded in an AAV vector in Hela cells.

FIG. 5 is a histogram showing the activity of the passenger strand of the modulatory polynucleotides encoded in an AAV vector in HeLa cells.

FIG. 6 is a histogram for the intracellular AAV DNA.

FIG. 7 is a histogram showing the activity of the constructs encoded in an AAV vector in human motor neurons.

FIG. 8 is a chart showing the dose-dependent silencing of SOD1 in U251MG cells.

FIG. 9 is a chart showing the dose-dependent silencing of SOD1 in human astrocyte cells.

FIG. 10 is a chart showing the time course of the silencing of SOD1 in U251MG cells.

FIG. 11A, FIG. 11B and FIG. 11C are charts showing the dose-dependent effects of a construct. FIG. 11A shows the relative SOD1 expression. FIG. 11B shows the percent of guide strand. FIG. 11C shows the percent of the passenger strand.

FIG. 12 is a diagram showing the location of the modulatory polynucleotide (MP) in relation to the ITRs, the intron (I) and the polyA (P).

### DETAILED DESCRIPTION OF THE INVENTION

The present invention relates to modulatory polynucleotides, e.g., RNA or DNA molecules as therapeutic agents. RNA interfering mediated gene silencing can specifically inhibit targeted gene expression. The present invention then provides small double stranded RNA (dsRNA) molecules (small interfering RNA, siRNA) targeting the SOD1 gene, pharmaceutical compositions comprising such siRNAs, as well as processes of their design. The present invention also provides methods of their use for inhibiting SOD1 gene expression and protein production, for treating neurodegenerative disease, in particular, amyotrophic lateral sclerosis (ALS).

The present invention provides small interfering RNA (siRNA) duplexes (and modulatory polynucleotides encoding them) that target SOD1 mRNA to interfere with SOD1 gene expression and/or SOD1 protein production. The siRNA duplexes of the present invention may interfere with both alleles of the SOD1 gene irrespective of any particular mutation in the SOD1 gene, and may particularly interact with those found in ALS disease.

In some embodiments, a nucleic acid sequence encoding such siRNA molecules, or a single strand of the siRNA molecules, is inserted into adeno-associated viral vectors and introduced into cells, specifically motor neurons and/or other surrounding cells in the central nervous system.

The encoded siRNA duplex of the present invention contains an antisense strand and a sense strand hybridized together forming a duplex structure, wherein the antisense strand is complementary to the nucleic acid sequence of the targeted SOD1 gene, and wherein the sense strand is homologous to the nucleic acid sequence of the targeted SOD1 gene. In some aspects, the 5′end of the antisense strand has a 5′ phosphate group and the 3′end of the sense strand contains a 3′hydroxyl group. In other aspects, there are none, one or 2 nucleotide overhangs at the 3′end of each strand.

According to the present invention, each strand of the siRNA duplex targeting the SOD1 gene is about 19 to 25, 19 to 24 or 19 to 21 nucleotides in length, preferably about 19 nucleotides, 20 nucleotides, 21 nucleotides, 22 nucleotides, 23 nucleotides, 24 nucleotides, or 25 nucleotides in length. In some aspects, the siRNAs may be unmodified RNA molecules.

In other aspects, the siRNAs may contain at least one modified nucleotide, such as base, sugar or backbone modification.

In one embodiment, an siRNA or dsRNA includes at least two sequences that are complementary to each other. The dsRNA includes a sense strand having a first sequence and an antisense strand having a second sequence. The antisense strand includes a nucleotide sequence that is substantially complementary to at least part of an mRNA encoding SOD1, and the region of complementarity is 30 nucleotides or less, and at least 15 nucleotides in length. Generally, the dsRNA is 19 to 25, 19 to 24 or 19 to 21 nucleotides in length. In some embodiments the dsRNA is from about 15 to about 25 nucleotides in length, and in other embodiments the dsRNA is from about 25 to about 30 nucleotides in length.

The dsRNA, whether directly administered or encoded in an expression vector upon contacting with a cell expressing SOD1, inhibits the expression of SOD1 by at least 10%, at least 20%, at least 25%, at least 30%, at least 35% or at least 40% or more, such as when assayed by a method as described herein.

The siRNA molecules included in the compositions featured herein comprise a dsRNA having an antisense strand (the antisense strand) having a region that is 30 nucleotides or less, generally 19 to 25, 19 to 24 or 19 to 21 nucleotides in length, that is substantially complementary to at least part of an mRNA transcript of the SOD1 gene.

According to the present invention, AAV vectors comprising the nucleic acids of the siRNA duplexes, one strand of the siRNA duplex or the dsRNA targeting SOD1 gene are produced, the AAV vector serotypes may be AAV1, AAV2, AAV3, AAV4, AAV5, AAV6, AAV7, AAV8, AAV9, AAV9.47, AAV9 (hu14), AAV10, AAV11, AAV12, AAVrh8, AAVrh10, AAV-DJ8 and AAV-DJ, and variants thereof.

According to the present invention, siRNA duplexes or the encoded dsRNA targeting the SOD1 gene in ALS is selected from the siRNA duplexes listed in Table 3. In some embodiments, the siRNA duplexes or dsRNA targeting the SOD1 gene in ALS is selected from the group consisting of siRNA duplexes: D-2757, D-2806, D-2860, D-2861, D-2875, D-2871, D-2758, D-2759, D-2866, D-2870, D-2823 and D-2858.

The present invention also provides pharmaceutical compositions comprising at least one siRNA duplex targeting the SOD1 gene and a pharmaceutically acceptable carrier. In some aspects, the siRNA duplex is encoded by an AAV vector.

In some embodiments, the present invention provides methods for inhibiting/silencing SOD1 gene expression in a cell. Accordingly, the siRNA duplexes or encoded dsRNA can be used to substantially inhibit SOD1 gene expression in a cell, in particular in a motor neuron. In some aspects, the inhibition of SOD1 gene expression refers to an inhibition by at least about 20%, such as by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. Accordingly, the protein product of the targeted gene may be inhibited by at least about 20%, preferably by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. The SOD1 gene can be either a wild type gene or a mutated SOD1 gene with at least one mutation. Accordingly, the SOD1 protein is either wild type protein or a mutated polypeptide with at least one mutation.

In one embodiment, the siRNA duplexes or encoded dsRNA may be used to reduce the expression of SOD1 protein by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of SOD1 protein expression may be reduced 50-90%.

In one embodiment, the siRNA duplexes or encoded dsRNA may be used to reduce the expression of SOD1 mRNA by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of SOD1 mRNA expression may be reduced 50-90%.

In one embodiment, the siRNA duplexes or encoded dsRNA may be used to reduce the expression of SOD1 protein and/or mRNA in at least one region of the CNS such as, but not limited to the spinal cord, the forebrain, the midbrain or the hindbrain. The expression of SOD1 protein and/or mRNA is reduced by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in at least one region of the CNS. As a non-limiting example, the expression of SOD1 protein and mRNA in the spinal cord is reduced by 50-90%.

In some embodiments, the present invention provides methods for treating, or ameliorating amyotrophic lateral sclerosis associated with abnormal SOD1 gene and/or SOD1 protein in a subject in need of treatment, the method comprising administering to the subject a pharmaceutically effective amount of at least one siRNA duplex or a nucleic acid encoding an siRNA duplex targeting the SOD1 gene, delivering said siRNA duplex (or encoded duplex) into targeted cells, inhibiting SOD1 gene expression and protein production, and ameliorating symptoms of ALS in the subject.

In some embodiments, an AAV vector comprising the nucleic acid sequence of at least one siRNA duplex targeting the SOD1 gene is administered to the subject in need for treating and/or ameliorating ALS. The AAV vector serotype may be selected from the group consisting of AAV1, AAV2, AAV3, AAV4, AAV5, AAV6, AAV7, AAV8, AAV9, AAV9.47, AAV9 (hu14), AAV10, AAV11, AAV12, AAVrh8, AAVrh10, AAV-DJ8 (AAVDJ8) and AAV-DJ (AAVDJ), and variants thereof. In one embodiment, the AAV vector serotype is AAV2. In another embodiment, the AAV vector is AAVDJ. In yet another embodiment, the AAV vector serotype is AAVDJ8.

In one embodiment, the serotype which may be useful in the present invention may be AAV-DJ8. The amino acid sequence of AAV-DJ8 may comprise two or more mutations in order to remove the heparin binding domain (HBD). As a non-limiting example, the AAV-DJ sequence described as SEQ ID NO: 1 in U.S. Pat. No. 7,588,772, the contents of which are herein incorporated by reference in their entirety, may comprise two mutations: (1) R587Q where arginine (R; arg) at amino acid 587 is changed to glutamine (Q; Gln) and (2) R590T where arginine (R; Arg) at amino acid 590 is changed to threonine (T; Thr). As another non-limiting example, may comprise three mutations: (1) K406R where lysine (K; Lys) at amino acid 406 is changed to arginine (R; Arg), (2) R587Q where arginine (R; Arg) at amino acid 587 is changed to glutamine (Q; Gln) and (3) R590T where arginine (R; Arg) at amino acid 590 is changed to threonine (T; Thr).

In some aspects, ALS is familial ALS linked to SOD1 mutations. In other aspects, ALS is sporadic ALS which is characterized by abnormal aggregation of SOD1 protein or abberations in SOD1 protein function and localization. The symptoms of ALS ameliorated by the present method may include, but are not limited to, motor neuron degeneration, muscle weakness, stiffness of muscles, slurred speech and/or difficulty in breathing.

In some embodiments, the siRNA duplexes or encoded dsRNA targeting the SOD1 gene or the AAV vectors comprising such siRNA molecules may be introduced directly into the central nervous system of the subject, for example, by intracranial injection.

In some embodiments, the pharmaceutical composition of the present invention is used as a solo therapy. In other embodiments, the pharmaceutical composition of the present invention is used in combination therapy. The combination therapy may be in combination with one or more neuroprotective agents such as small molecule compounds, growth factors and hormones which have been tested for their neuroprotective effect on motor neuron degeneration.

In some embodiments, the present invention provides methods for treating, or ameliorating amyotrophic lateral sclerosis by administering to a subject in need thereof a therapeutically effective amount of a plasmid or AAV vector described herein. The ALS may be familial ALS or sporadic ALS.

The details of one or more embodiments of the invention are set forth in the accompanying description below. Although any materials and methods similar or equivalent to those described herein can be used in the practice or testing of the present invention, the preferred materials and methods are now described. Other features, objects and advantages of the invention will be apparent from the description. In the description, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present description will control.

### Amyotrophic Lateral Sclerosis (ALS)

Amyotrophic lateral sclerosis (ALS), an adult-onset neurodegenerative disorder, is a progressive and fatal disease characterized by the selective death of motor neurons in the motor cortex, brainstem and spinal cord. The incidence of ALS is about 1.9 per 100,000. Patients diagnosed with ALS develop a progressive muscle phenotype characterized by spasticity, hyperreflexia or hyporeflexia, fasciculations, muscle atrophy and paralysis. These motor impairments are caused by the denervation of muscles due to the loss of motor neurons. The major pathological features of ALS include degeneration of the corticospinal tracts and extensive loss of lower motor neurons (LMNs) or anterior horn cells (Ghatak et al., J Neuropathol Exp Neurol., 1986, 45, 385-395), degeneration and loss of Betz cells and other pyramidal cells in the primary motor cortex (Udaka et al., Acta Neuropathol, 1986, 70, 289-295; Maekawa et al., Brain, 2004, 127, 1237-1251) and reactive gliosis in the motor cortex and spinal cord (Kawamata et al., Am J Pathol., 1992, 140,691-707; and Schiffer et al., J Neurol Sci., 1996, 139, 27-33). ALS is usually fatal within 3 to 5 years after the diagnosis due to respiratory defects and/or inflammation (Rowland L P and Shneibder N A, N Engl. J. Med., 2001, 344, 1688-1700).

A cellular hallmark of ALS is the presence of proteinaceous, ubiquitinated, cytoplasmic inclusions in degenerating motor neurons and surrounding cells (e.g., astrocytes). Ubiquitinated inclusions (i.e., Lewy body-like inclusions or Skein-like inclusions) are the most common and specific type of inclusion in ALS and are found in LMNs of the spinal cord and brainstem, and in corticospinal upper motor neurons (UMNs) (Matsumoto et al., J Neurol Sci., 1993, 115, 208-213; and Sasak and Maruyama, Acta Neuropathol., 1994, 87, 578-585). A few proteins have been identified to be components of the inclusions, including ubiquitin, Cu/Zn superoxide dismutase 1 (SOD1), peripherin and Dorfin. Neurofilamentous inclusions are often found in hyaline conglomerate inclusions (HCIs) and axonal ‘spheroids’ in spinal cord motor neurons in ALS. Other types and less specific inclusions include Bunina bodies (cystatin C-containing inclusions) and Crescent shaped inclusions (SCIs) in upper layers of the cortex. Other neuropathological features seen in ALS include fragmentation of the Golgi apparatus, mitochondrial vacuolization, and ultrastructural abnormalities of synaptic terminals (Fujita et al., Acta Neuropathol. 2002, 103, 243-247).

In addition, in frontotemporal dementia ALS (FTD-ALS) cortical atrophy (including the frontal and temporal lobes) is also observed, which may cause cognitive impairment in FTD-ALS patients.

ALS is a complex and multifactorial disease and multiple mechanisms hypothesized as responsible for ALS pathogenesis include, but are not limited to, dysfunction of protein degradation, glutamate excitotoxicity, mitochondrial dysfunction, apoptosis, oxidative stress, inflammation, protein misfolding and aggregation, aberrant RNA metabolism, and altered gene expression.

About 10%-15% of ALS cases have family history of the disease, and these patients are referred to as familial ALS (fALS) or inherited patients, commonly with a Mendelian dominant mode of inheritance and high penetrance. The remainder (approximately 85%-95%) is classified as sporadic ALS (sALS), as they are not associated with a documented family history, but instead are thought to be due to other risk factors including, but not limited to environmental factors, genetic polymorphisms, somatic mutations, and possibly gene-environmental interactions. In most cases, familial (or inherited) ALS is inherited as autosomal dominant disease, but pedigrees with autosomal recessive and X-linked inheritance and incomplete penetrance exist. Sporadic and familial forms are clinically indistinguishable suggesting a common pathogenesis. The precise cause of the selective death of motor neurons in ALS remains elusive. Progress in understanding the genetic factors in fALS may shed light on both forms of the disease.

Recently, an explosion to genetic causes of ALS has discovered mutations in more than 10 different genes that are known to cause fALS. The most common ones are found in the genes encoding Cu/Zn superoxide dismutase 1 (SOD1; ˜20%) (Rosen D R et al., Nature, 1993, 362, 59-62), fused in sarcoma/translated in liposarcoma (FUS/TLS; 1-5%) and TDP-43 (TARDBP; 1-5%). Recently, a hexanucleotide repeat expansion (GGGGCC), in the C9orF72 gene was identified as the most frequent cause of fALS (˜40%) in the Western population (reviewed by Renton et al., Nat. Neurosci., 2014, 17, 17-23). Other genes mutated in ALS include alsin (ALS2), senataxin (SETX), vesicle-associated membrane protein (VAPB), and angiogenin (ANG). fALS genes control different cellular mechanisms, suggesting that the pathogenesis of ALS is complicated and may be related to several different processes finally leading to motor neuron degeneration.

SOD1 is one of the three human superoxide dismutases identified and characterized in mammals: copper-zinc superoxide dismutase (Cu/ZnSOD or SOD1), manganese superoxide dismutase (MnSOD or SOD2), and extracellular superoxide dismutase (ECSOD or SOD3). SOD1 is a 32 kDa homodimer of a 153-residue polypeptide with one copper- and one zinc-binding site per subunit, which is encoded by the SOD1 gene (GeneBank access No.: NM_000454.4) on human chromosome 21 (see Table 2). SOD1 catalyzes the reaction of superoxide anion (O²⁻) into molecular oxygen (O₂) and hydrogen peroxide (H₂O₂) at a bound copper ion. The intracellular concentration of SOD1 is high (ranging from 10 to 100 μM), accounting for 1% of the total protein content in the central nervous system (CNS). The protein is localized not only in the cytoplasm but also in the nucleus, lysosomes, peroxisomes, and mitochondrial intermembrane spaces in eukaryotic cells (Lindenau J et al., Glia, 2000, 29, 25-34).

Mutations in the SOD1gene are carried by 15-20% of fALS patients and by 1-2% of all ALS cases. Currently, at least 170 different mutations distributed throughout the 153-amino acid SOD1 polypeptide have been found to cause ALS, and an updated list can be found at the ALS online Genetic Database (ALSOD) (Wroe R et al., Amyotroph Lateral Scler., 2008, 9, 249-250). Table 1 lists some examples of mutations in SOD1 in ALS. These mutations are predominantly single amino acid substitutions (i.e. missense mutations) although deletions, insertions, and C-terminal truncations also occur. Different SOD1 mutations display different geographic distribution patterns. For instance, 40-50% of all Americans with ALS caused by SOD1 gene mutations have a particular mutation Ala4Val (or A4V). The A4V mutation is typically associated with more severe signs and symptoms and the survival period is typically 2-3 years. The I113T mutation is by far the most common mutation in the United Kingdom. The most prevalent mutation in Europe is D90A substitute and the survival period is usually greater than 10 years.

TABLE 1
Examples of SOD1 mutations in ALS

| Location | Mutations |
|---|---|
| Exon1 | Q22L; E21K, G; F20C; N19S; G16A, S; V14M, S; G12R; |
| (220 bp) | G10G, V, R; L8Q, V; V7E; C6G, F; V5L; A4T, V, S |
| Exon2 | T54R; E49K; H48R, Q; V47F, A; H46R; F45C; H43R; |
| (97 bp) | G41S, D; G37R; V29, insA |
| Exon3 | D76Y, V; G72S, C; L67R; P66A; N65S; S59I, S |
| (70 bp) | |
| Exon4 | D124G, V; V118L, InsAAAAC; L117V; T116T; R115G; |
| (118 bp) | G114A; I113T, F; I112M, T; G108V; L106V, F; |
| | S106L, delTCACTC; I104F; D101G, Y, H, N; E100G, K; |
| | I99V; V97L, M; D96N, V; A95T, V; G93S, V, A, C, R, D; |
| | D90V, A; A89T, V; T88delACTGCTGAC; V87A, M; |
| | N86I, S, D, K; G85R, S; L84V, F; H80R |
| Exon5 | I151T, S; I149T; V148I, G; G147D, R; C146R, stop; |
| (461 bp) | A145T, G; L144F, S; G141E, stop; A140A, G; |
| | N139D, K, H, N; G138E; T137R; S134N; |
| | E133V, delGAA, insTT; E132insTT; G127R, InsTGGG; |
| | L126S, delITT, stop; D126, delTT |

To investigate the mechanism of neuronal death associated with SOD1 gene defects, several rodent models of SOD1-linked ALS were developed in the art, which express the human SOD1 gene with different mutations, including missense mutations, small deletions, or insertions. Non-limiting examples of ALS mouse models include SOD1^{G93A}, SOD1^{A4V}, SOD1^{G37R}, SOD1^{G85R}, SOD1^{D90A}, SOD1^{L84V}, SOD1^{I113T}, SOD1^{H36R/H480}, SOD1^{G127X}, SOD1^{L126X} and SOD1^{L126delTT} There are two transgenic rat models carrying two different human SOD1 mutations: SOD1^{H46R} and SOD1^{G93R}. These rodent ALS models can develop muscle weakness similar to human ALS patients and other pathogenic features that reflect several characteristics of the human disease, in particular, the selective death of spinal motor neurons, aggregation of protein inclusions in motor neurons and microglial activation. It is well known in the art that the transgenic rodents are good models of human SOD1-associated ALS disease and provide models for studying disease pathogenesis and developing disease treatment.

Studies in animal and cellular models showed that SOD1 pathogenic variants cause ALS by gain of function. That is to say, the superoxide dismutase enzyme gains new but harmful properties when altered by SOD1 mutations. For example, some SOD1 mutated variants in ALS increase oxidative stress (e.g., increased accumulation of toxic superoxide radicals) by disrupting the redox cycle. Other studies also indicate that some SOD1 mutated variants in ALS might acquire toxic properties that are independent of its normal physiological function (such as abnormal aggregation of misfolded SOD1 variants. In the aberrant redox chemistry model, mutant SOD1 is unstable and through aberrant chemistry interacts with nonconventional substrates causing overproduction of reactive oxygen species (ROS). In the protein toxicity model, unstable, misfolded SOD1 aggregates into cytoplasmic inclusion bodies, sequestering proteins crucial for cellular processes. These two hypotheses are not mutually exclusive. It has been shown that oxidation of selected histidine residues that bind metals in the active site mediates SOD1 aggregation.

The aggregated mutant SOD1 protein may also induce mitochondrial dysfunction (Vehvilainen P et al., Front Cell Neurosci., 2014, 8, 126), impairment of axonal transport, aberrant RNA metabolism, glial cell pathology and glutamate excitotoxicity. In some sporadic ALS cases, misfolded wild-type SOD1 protein is found in diseased motor neurons which forms a “toxic conformation” that is similar to that which is seen with familial ALS-linked SOD1 variants (Rotunno M S and Bosco D A, Front Cell Neurosci., 2013, 16, 7, 253). Such evidence suggests that ALS is a protein folding diseases analogous to other neurodegenerative diseases such as Alzheimer's disease and Parkinson's disease.

Currently, no curative treatments are available for patients suffering from ALS. The only FDA approved drug Riluzole, an inhibitor of glutamate release, has a moderate effect on ALS, only extending survival by 2-3 months if it is taken for 18 months. Unfortunately, patients taking riluzole do not experience any slowing in disease progression or improvement in muscle function. Therefore, riluzole does not present a cure, or even an effective treatment. Researchers continue to search for better therapeutic agents.

Therapeutic approaches that may prevent or ameliorate SOD1 aggregation have been tested previously. For example, arimoclomol, a hydroxylamine derivative, is a drug that targets heat shock proteins, which are cellular defense mechanisms against these aggregates. Studies demonstrated that treatment with arimoclomol improved muscle function in SOD1 mouse models. Other drugs that target one or more cellular defects in ALS may include AMPA antagonists such as talampanel, beta-lactam antibiotics, which may reduce glutamate-induced excitotoxicity to motor neurons; Bromocriptine that may inhibit oxidative induced motor neuron death (e.g. U.S. Patent publication No. 20110105517; the content of which is incorporated herein by reference in its entirety); 1,3-diphenylurea derivative or multikinase inhibitor which may reduce SOD1 gene expression (e.g., U.S. Patent Publication No. 20130225642; the content of which is incorporated herein by reference in its entirety); dopamine agonist pramipexole and its anantiomer dexpramipexole, which may ameliorate the oxidative response in mitochondria; nimesulide, which inhibits cyclooxygenase enzyme (e.g., U.S. Patent Publication No. 20060041022; the content of which is incorporated herein by reference in its entirety); drugs that act as free radical scavengers (e.g. U.S. Pat. No. 6,933,310 and PCT Patent Publication No.: WO2006075434; the content of each of which is incorporated herein by reference in their entirety).

Another approach to inhibit abnormal SOD1 protein aggregation is to silence/inhibit SOD1 gene expression in ALS. It has been reported that small interfering RNAs for specific gene silencing of the mutated allele are therapeutically beneficial for the treatment of fALS (e.g., Ralgh G S et al., Nat. Medicine, 2005, 11 (4), 429-433; and Raoul C et al., Nat. Medicine, 2005, 11 (4), 423-428; and Maxwell M M et al., PNAS, 2004, 101 (9), 3178-3183; and Ding H et al., Chinese Medical J., 2011, 124 (1), 106-110; and Scharz D S et al., Plos Genet., 2006, 2 (9), e140; the content of each of which is incorporated herein by reference in their entirety).

Many other RNA therapeutic agents that target the SOD1 gene and modulate SOD1 expression in ALS are taught in the art. Such RNA based agents include antisense oligonucleotides and double stranded small interfering RNAs. See, e.g., Wang H et al., J Biol. Chem., 2008, 283 (23), 15845-15852); U.S. Pat. Nos. 7,498,316; 7,632,938; 7,678,895; 7,951,784; 7,977,314; 8,183,219; 8,309,533 and 8, 586, 554; and U.S. Patent publication Nos. 2006/0229268 and 2011/0263680; the content of each of which is herein incorporated by reference in their entirety.

The present invention provides modulatory polynucleotides, e.g., siRNA molecules targeting the SOD1 gene and methods for their design and manufacture. Particularly, the present invention employs viral vectors such as adeno-associated viral (AAV) vectors comprising the nucleic acid sequence encoding the siRNA molecules of the present invention. The AAV vectors comprising the nucleic acid sequence encoding the siRNA molecules of the present invention may increase the delivery of active agents into motor neurons. The siRNA duplexes or encoding dsRNA targeting the SOD1 gene may be able to inhibit SOD1 gene expression (e.g., mRNA level) significantly inside cells; therefore, ameliorating SOD1 expression induced stress inside the cells such as aggregation of protein and formation of inclusions, increased free radicals, mitochondrial dysfunction, and RNA metabolism.

Such siRNA mediated SOD1 expression inhibition may be used for treating ALS. According to the present invention, methods for treating and/or ameliorating ALS in a patient comprises administering to the patient an effective amount of AAV vector comprising a nucleic acid sequence encoding the siRNA molecules of the present invention into cells. The administration of the AAV vector comprising such a nucleic acid sequence will encode the siRNA molecules which cause the inhibition/silence of SOD1 gene expression.

In one embodiment, the vectors, e.g., AAV encoding the modulatory polynucleotide, reduce the expression of mutant SOD1 in a subject. The reduction of mutant SOD1 can also reduce the formation of toxic aggregates which can cause mechanisms of toxicity such as, but not limited to, oxidative stress, mitochondrial dysfunction, impaired axonal transport, aberrant RNA metabolism, glial cell pathology and/or glutamate excitotoxicity.

In one embodiment, the vector, e.g., AAV vectors, reduces the amount of SOD1 in a subject in need thereof and thus provides a therapeutic benefit as described herein.

### Compositions of the Invention

siRNA Molecules

The present invention relates to RNA interference (RNAi) induced inhibition of gene expression for treating neurodegenerative disorders. Provided herein are siRNA duplexes or encoded dsRNA that target the SOD1 gene (referred to herein collectively as “siRNA molecules”). Such siRNA duplexes or encoded dsRNA can reduce or silence SOD1 gene expression in cells, for example, motor neurons, thereby, ameliorating symptoms of ALS such as, but not limited to, motor neuron death and muscle atrophy.

RNAi (also known as post-transcriptional gene silencing (PTGS), quelling, or co-suppression) is a post-transcriptional gene silencing process in which RNA molecules, in a sequence specific manner, inhibit gene expression, typically by causing the destruction of specific mRNA molecules. The active components of RNAi are short/small double stranded RNAs (dsRNAs), called small interfering RNAs (siRNAs), that typically contain 15-30 nucleotides (e.g., 19 to 25, 19 to 24 or 19-21 nucleotides) and 2 nucleotide 3′ overhangs and that match the nucleic acid sequence of the target gene. These short RNA species may be naturally produced in vivo by Dicer-mediated cleavage of larger dsRNAs and they are functional in mammalian cells.

Naturally expressed small RNA molecules, named microRNAs (miRNAs), elicit gene silencing by regulating the expression of mRNAs. The miRNAs containing RNA Induced Silencing Complex (RISC) targets mRNAs presenting a perfect sequence complementarity with nucleotides 2-7 in the 5′region of the miRNA which is called the seed region, and other base pairs with its 3′region. miRNA mediated down regulation of gene expression may be caused by cleavage of the target mRNAs, translational inhibition of the target mRNAs, or mRNA decay. miRNA targeting sequences are usually located in the 3′-UTR of the target mRNAs. A single miRNA may target more than 100 transcripts from various genes, and one mRNA may be targeted by different miRNAs.

siRNA duplexes or dsRNA targeting a specific mRNA may be designed and synthesized in vitro and introduced into cells for activating RNAi processes. Elbashir et al. demonstrated that 21-nucleotide siRNA duplexes (termed small interfering RNAs) were capable of effecting potent and specific gene knockdown without inducing immune response in mammalian cells (Elbashir S M et al., Nature, 2001, 411, 494-498). Since this initial report, post-transcriptional gene silencing by siRNAs quickly emerged as a powerful tool for genetic analysis in mammalian cells and has the potential to produce novel therapeutics.

In vitro synthetized siRNA molecules may be introduced into cells in order to activate RNAi. An exogenous siRNA duplex, when it is introduced into cells, similar to the endogenous dsRNAs, can be assembled to form the RNA Induced Silencing Complex (RISC), a multiunit complex that facilitates searching through the genome for RNA sequences that are complementary to one of the two strands of the siRNA duplex (i.e., the antisense strand). During the process, the sense strand (or passenger strand) of the siRNA is lost from the complex, while the antisense strand (or guide strand) of the siRNA is matched with its complementary RNA. In particular, the targets of siRNA containing RISC complex are mRNAs presenting a perfect sequence complementarity. Then, siRNA mediated gene silencing occurs, cleaving, releasing and degrading the target.

The siRNA duplex comprised of a sense strand homologous to the target mRNA and an antisense strand that is complementary to the target mRNA offers much more advantage in terms of efficiency for target RNA destruction compared to the use of the single strand (ss)-siRNAs (e.g. antisense strand RNA or antisense oligonucleotides). In many cases it requires higher concentration of the ss-siRNA to achieve the effective gene silencing potency of the corresponding duplex.

Any of the foregoing molecules may be encoded by an AAV vector or vector genome.

Design and Sequences of siRNA Duplexes Targeting SOD1 Gene

Some guidelines for designing siRNAs have been proposed in the art. These guidelines generally recommend generating a 19-nucleotide duplexed region, symmetric 2-3 nucleotide 3′overhangs, 5-phosphate and 3-hydroxyl groups targeting a region in the gene to be silenced. Other rules that may govern siRNA sequence preference include, but are not limited to, (i) A/U at the 5′ end of the antisense strand; (ii) G/C at the 5′ end of the sense strand; (iii) at least five A/U residues in the 5′ terminal one-third of the antisense strand; and (iv) the absence of any GC stretch of more than 9 nucleotides in length. In accordance with such consideration, together with the specific sequence of a target gene, highly effective siRNA molecules essential for suppressing mammalian target gene expression may be readily designed.

According to the present invention, siRNA molecules (e.g., siRNA duplexes or encoded dsRNA) that target the human SOD1 gene are designed. Such siRNA molecules can specifically, suppress SOD1 gene expression and protein production. In some aspects, the siRNA molecules are designed and used to selectively “knock out” SOD1 gene variants in cells, i.e., mutated SOD1 transcripts that are identified in patients with ALS disease (e.g., mutations in Table1). In some aspects, the siRNA molecules are designed and used to selectively “knock down” SOD1 gene variants in cells. In other aspects, the siRNA molecules are able to inhibit or suppress both wild type and mutated alleles of SOD1 gene irrelevant of any particular mutations in the SOD1 gene.

In one embodiment, an siRNA molecule of the present invention comprises a sense strand and a complementary antisense strand in which both strands are hybridized together to form a duplex structure. The antisense strand has sufficient complementarity to the SOD1 mRNA sequence to direct target-specific RNAi, i.e., the siRNA molecule has a sequence sufficient to trigger the destruction of the target mRNA by the RNAi machinery or process.

In some embodiments, the antisense strand and target mRNA sequences are 100% complementary. The antisense strand may be complementary to any part of the target mRNA sequence.

In other embodiments, the antisense strand and target mRNA sequences comprise at least one mismatch. As a non-limiting example, the antisense strand and the target mRNA sequence are at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98% or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% complementary.

According to the present invention, the siRNA molecule has a length from about 10-50 or more nucleotides, i.e., each strand comprising 10-50 nucleotides (or nucleotide analogs). Preferably, the siRNA molecule has a length from about 15-30, e.g., 15, 16, 17, 18, 19, 20, 21, 22, 23, 24, 25, 26, 27, 28, 29, or 30 nucleotides in each strand, wherein one of the strands is sufficiently complementary to a target region. In one embodiment, the siRNA molecule has a length from about 19 to 25, 19 to 24 or 19 to 21 nucleotides.

In some embodiments, the siRNA molecules of the present invention can be synthetic RNA duplexes comprising about 19 nucleotides to about 25 nucleotides, and two overhanging nucleotides at the 3′-end. In some aspects, the siRNA molecules may be unmodified RNA molecules. In other aspects, the siRNA molecules may contain at least one modified nucleotide, such as base, sugar or backbone modifications.

In other embodiments, the siRNA molecules of the present invention can be encoded in plasmid vectors, viral vectors (e.g., AAV vectors), genome or other nucleic acid expression vectors for delivery to a cell.

DNA expression plasmids can be used to stably express the siRNA duplexes or dsRNA of the present invention in cells and achieve long-term inhibition of the target gene. In one aspect, the sense and antisense strands of a siRNA duplex are typically linked by a short spacer sequence leading to the expression of a stem-loop structure termed short hairpin RNA (shRNA). The hairpin is recognized and cleaved by Dicer, thus generating mature siRNA molecules.

According to the present invention, AAV vectors comprising the nucleic acids encoding the siRNA molecules targeting SOD1 mRNA are produced, the AAV vector serotypes may be AAV1, AAV2, AAV3, AAV4, AAV5, AAV6, AAV7, AAV8, AAV9, AAV9.47, AAV9 (hu14), AAV10, AAV11, AAV12, AAVrh8, AAVrh10, AAV-DJ8 and AAV-DJ, and variants thereof.

In some embodiments, the siRNA duplexes or encoded dsRNA of the present invention suppress (or degrade) target mRNA (i.e., SOD1). Accordingly, the siRNA duplexes or encoded dsRNA can be used to substantially inhibit SOD1 gene expression in a cell, for example a motor neuron. In some aspects, the inhibition of SOD1 gene expression refers to an inhibition by at least about 20%, preferably by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. Accordingly, the protein product of the targeted gene may be inhibited by at least about 20%, preferably by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. The SOD1 gene can be either a wild type gene or a mutated SOD1 gene with at least one mutation. Accordingly, the protein is either wild type protein or a mutated polypeptide with at least one mutation.

According to the present invention, siRNA duplexes or encoded dsRNA targeting human SOD1 gene were designed and tested for their ability in reducing SOD1 mRNA levels in cultured cells. Such siRNA duplexes include those listed in Table 3. As a non-limiting example, the siRNA duplexes may be siRNA duplex IDs: D-2757, D-2806, D-2860, D-2861, D-2875, D-2871, D-2758, D-2759, D-2866, D-2870, D-2823 and D-2858.

In one embodiment, the 3′ stem arm of the siRNA duplexes or encoded dsRNA targeting the human SOD1 gene may have 11 nucleotides downstream of the 3′ end of the guide strand which have complementarity to the 11 of the 13 nucleotides upstream of the 5′ end of the passenger strand in the 5′ stem arm.

In one embodiment, the siRNA duplexes or encoded dsRNA targeting human SOD1 gene may have a cysteine which is 6 nucleotides downstream of the 3′ end of the 3′ stem arm of the modulatory polynucleotide.

In one embodiment, the siRNA duplexes or encoded dsRNA targeting human SOD1 gene comprise a miRNA seed match for the guide strand. In another embodiment, the siRNA duplexes or encoded dsRNA targeting human SOD1 gene comprise a miRNA seed match for the passenger strand. In yet another embodiment, the siRNA duplexes or encoded dsRNA targeting human SOD1 gene do not comprise a seed match for the guide or passenger strand.

In one embodiment, the siRNA duplexes or encoded dsRNA targeting human SOD1 gene may have almost no significant full-length off targets for the guide strand. In another embodiment, the siRNA duplexes or encoded dsRNA targeting human SOD1 gene may have almost no significant full-length off targets for the passenger strand. The siRNA duplexes or encoded dsRNA targeting human SOD1 gene may have less than 1%, 2%, 3%, 4%, 5%, 6%, 7%, 8%, 9%, 10%, 1-5%, 2-6%, 3-7%, 4-8%, 5-9%, 5-10% 6-10% full-length off targets for the passenger strand. In yet another embodiment, the siRNA duplexes or encoded dsRNA targeting human SOD1 gene may have almost no significant full-length off targets for the guide strand or the passenger strand. The siRNA duplexes or encoded dsRNA targeting human SOD1 gene may have less than 1%, 2%, 3%, 4%, 5%, 6%, 7%, 8%, 9%, 10%, 1-5%, 2-6%, 3-7%, 4-8%, 5-9%, 5-10% 6-10% full-length off targets for the guide or passenger strand.

In one embodiment, the siRNA duplexes or encoded dsRNA targeting human SOD1 gene may have high activity in vitro. In another embodiment, the siRNA duplexes or encoded dsRNA targeting the human SOD1 gene may have low activity in vitro. In yet another embodiment, the siRNA duplexes or dsRNA targeting the human SOD1 gene may have high guide strand activity and low passenger strand activity in vitro.

In one embodiment, the siRNA duplexes or encoded dsRNA targeting the human SOD1 gene have a high guide strand activity and low passenger strand activity in vitro. The target knock-down (KD) by the guide strand may be at least 60%, 65%, 70%, 75%, 80%, 85%, 90%, 95%, 99%, 99.5% or 100%. The target knock-down by the guide strand may be 60-65%, 60-70%, 60-75%, 60-80%, 60-85%, 60-90%, 60-95%, 60-99%, 60-99.5%, 60-100%, 65-70%, 65-75%, 65-80%, 65-85%, 65-90%, 65-95%, 65-99%, 65-99.5%, 65-100%, 70-75%, 70-80%, 70-85%, 70-90%, 70-95%, 70-99%, 70-99.5%, 70-100%, 75-80%, 75-85%, 75-90%, 75-95%, 75-99%, 75-99.5%, 75-100%, 80-85%, 80-90%, 80-95%, 80-99%, 80-99.5%, 80-100%, 85-90%, 85-95%, 85-99%, 85-99.5%, 85-100%, 90-95%, 90-99%, 90-99.5%, 90-100%, 95-99%, 95-99.5%, 95-100%, 99-99.5%, 99-100% or 99.5-100%. As a non-limiting example, the target knock-down (KD) by the guide strand is greater than 70%.

In one embodiment, the IC₅₀ of the passenger strand for the nearest off target is greater than 100 multiplied by the IC₅₀ of the guide strand for the target. As a non-limiting example, if the IC₅₀ of the passenger strand for the nearest off target is greater than 100 multiplied by the IC₅₀ of the guide strand for the target then the siRNA duplexes or encoded dsRNA targeting the human SOD1 gene is said to have high guide strand activity and a low passenger strand activity in vitro.

In one embodiment, the 5′ processing of the guide strand has a correct start (n) at the 5′ end at least 75%, 80%, 85%, 90%, 95%, 99% or 100% of the time in vitro or in vivo. As a non-limiting example, the 5′ processing of the guide strand is precise and has a correct start (n) at the 5′ end at least 99% of the time in vitro. As a non-limiting example, the 5′ processing of the guide strand is precise and has a correct start (n) at the 5′ end at least 99% of the time in vivo.

In one embodiment, the guide-to-passenger (G:P) strand ratio expressed is 1:99, 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, 95:5, or 99:1 in vitro or in vivo. As a non-limiting example, the guide-to-passenger strand ratio is 80:20 in vitro. As a non-limiting example, the guide-to-passenger strand ratio is 80:20 in vivo.

In one embodiment, the integrity of the vector genome encoding the dsRNA is at least 60%, 65%, 70%, 75%, 80%, 85%, 90%, 95%, 99% or more than 99% of the full length of the construct.

siRNA Modification

In some embodiments, the siRNA molecules of the present invention, when not delivered as a precursor or DNA, may be chemically modified to modulate some features of RNA molecules, such as, but not limited to, increasing the stability of siRNAs in vivo. The chemically modified siRNA molecules can be used in human therapeutic applications, and are improved without compromising the RNAi activity of the siRNA molecules. As a non-limiting example, the siRNA molecules modified at both the 3′ and the 5′ end of both the sense strand and the antisense strand.

In some aspects, the siRNA duplexes of the present invention may contain one or more modified nucleotides such as, but not limited to, sugar modified nucleotides, nucleobase modifications and/or backbone modifications. In some aspects, the siRNA molecule may contain combined modifications, for example, combined nucleobase and backbone modifications.

In one embodiment, the modified nucleotide may be a sugar-modified nucleotide. Sugar modified nucleotides include, but are not limited to 2′-fluoro, 2′-amino and 2′-thio modified ribonucleotides, e.g., 2′-fluoro modified ribonucleotides. Modified nucleotides may be modified on the sugar moiety, as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles.

In one embodiment, the modified nucleotide may be a nucleobase-modified nucleotide.

In one embodiment, the modified nucleotide may be a backbone-modified nucleotide. In some embodiments, the siRNA duplexes of the present invention may further comprise other modifications on the backbone. A normal “backbone”, as used herein, refers to the repeatingly alternating sugar-phosphate sequences in a DNA or RNA molecule. The deoxyribose/ribose sugars are joined at both the 3′-hydroxyl and 5′-hydroxyl groups to phosphate groups in ester links, also known as “phosphodiester” bonds/linker (PO linkage). The PO backbones may be modified as “phosphorothioate backbone (PS linkage). In some cases, the natural phosphodiester bonds may be replaced by amide bonds but the four atoms between two sugar units are kept. Such amide modifications can facilitate the solid phase synthesis of oligonucleotides and increase the thermodynamic stability of a duplex formed with siRNA complement. See e.g., Mesmaeker et al., Pure & Appl. Chem., 1997, 3, 437-440; the content of which is incorporated herein by reference in its entirety.

Modified bases refer to nucleotide bases such as, for example, adenine, guanine, cytosine, thymine, uracil, xanthine, inosine, and queuosine that have been modified by the replacement or addition of one or more atoms or groups. Some examples of modifications on the nucleobase moieties include, but are not limited to, alkylated, halogenated, thiolated, aminated, amidated, or acetylated bases, individually or in combination. More specific examples include, for example, 5-propynyluridine, 5-propynylcytidine, 6-methyladenine, 6-methylguanine, N,N-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides.

In one embodiment, the modified nucleotides may be on just the sense strand.

In another embodiment, the modified nucleotides may be on just the antisense strand.

In some embodiments, the modified nucleotides may be in both the sense and antisense strands.

In some embodiments, the chemically modified nucleotide does not affect the ability of the antisense strand to pair with the target mRNA sequence, such as the SOD1 mRNA sequence.

### Vectors

In some embodiments, the siRNA molecules described herein can be encoded by vectors such as plasmids or viral vectors. In one embodiment, the siRNA molecules are encoded by viral vectors. Viral vectors may be, but are not limited to, Herpesvirus (HSV) vectors, retroviral vectors, adenoviral vectors, adeno-associated viral vectors, lentiviral vectors, and the like. In some specific embodiments, the viral vectors are AAV vectors.

### Retroviral Vectors

In some embodiments, the siRNA duplex targeting SOD1 gene may be encoded by a retroviral vector (See, e.g., U.S. Pat. Nos. 5,399,346; 5,124,263; 4,650,764 and 4,980,289; the content of each of which is incorporated herein by reference in their entirety).

### Adenoviral Vectors

Adenoviruses are eukaryotic DNA viruses that can be modified to efficiently deliver a nucleic acid to a variety of cell types in vivo, and have been used extensively in gene therapy protocols, including for targeting genes to neural cells. Various replication defective adenovirus and minimum adenovirus vectors have been described for nucleic acid therapeutics (See, e.g., PCT Patent Publication Nos. WO199426914, WO 199502697, WO199428152, WO199412649, WO199502697 and WO199622378; the content of each of which is incorporated by reference in their entirety). Such adenoviral vectors may also be used to deliver siRNA molecules of the present invention to cells.

### Adeno-Associated Viral (AAV) Vectors

An adeno-associated virus (AAV) is a dependent parvovirus (like other parvoviruses) which is a single stranded non-enveloped DNA virus having a genome of about 5000 nucleotides in length and which contains two open reading frames encoding the proteins responsible for replication (Rep) and the structural protein of the capsid (Cap). The open reading frames are flanked by two Inverted Terminal Repeat (ITR) sequences, which serve as the origin of replication of the viral genome. Furthermore, the AAV genome contains a packaging sequence, allowing packaging of the viral genome into an AAV capsid. The AAV vector requires a co-helper (e.g., adenovirus) to undergo productive infection in infected cells. In the absence of such helper functions, the AAV virions essentially enter host cells and integrate into the cells' genome.

AAV vectors have been investigated for siRNA delivery because of several unique features. Non-limiting examples of the features include (i) the ability to infect both dividing and non-dividing cells; (ii) a broad host range for infectivity, including human cells; (iii) wild-type AAV has not been associated with any disease and has not been shown to replicate in infected cells; (iv) the lack of cell-mediated immune response against the vector and (v) the non-integrative nature in a host chromosome thereby reducing potential for long-term expression. Moreover, infection with AAV vectors has minimal influence on changing the pattern of cellular gene expression (Stilwell and Samulski et al., Biotechniques, 2003, 34, 148).

Typically, AAV vectors for siRNA delivery may be recombinant viral vectors which are replication defective as they lack sequences encoding functional Rep and Cap proteins within the viral genome. In some cases, the defective AAV vectors may lack most or all coding sequences and essentially only contains one or two AAV ITR sequences and a packaging sequence.

AAV vectors may also comprise self-complementary AAV vectors (scAAVs). scAAV vectors contain both DNA strands which anneal together to form double stranded DNA. By skipping second strand synthesis, scAAVs allow for rapid expression in the cell.

In one embodiment, the AAV vector used in the present invention is a scAAV.

In one embodiment, the AAV vector used in the present invention is an ssAAV.

Methods for producing and/or modifying AAV vectors are disclosed in the art such as pseudotyped AAV vectors (PCT Patent Publication Nos. WO200028004; WO200123001; WO2004112727; WO 2005005610 and WO 2005072364, the content of each of which is incorporated herein by reference in their entirety).

AAV vectors comprising the nucleic acid sequence for the siRNA molecules may be prepared or derived from various serotypes of AAVs, including, but not limited to, AAV1, AAV2, AAV3, AAV4, AAV5, AAV6, AAV7, AAV8, AAV9, AAV9.47, AAV9 (hu14), AAV10, AAV11, AAV12, AAVrh8, AAVrh10, AAV-DJ8 and AAV-DJ. In some cases, different serotypes of AAVs may be mixed together or with other types of viruses to produce chimeric AAV vectors.

In one embodiment, the AAV vectors comprising a nucleic acid sequence encoding the siRNA molecules of the present invention may be introduced into mammalian cells.

AAV vectors may be modified to enhance the efficiency of delivery. Such modified AAV vectors comprising the nucleic acid sequence encoding the siRNA molecules of the present invention can be packaged efficiently and can be used to successfully infect the target cells at high frequency and with minimal toxicity.

In some embodiments, the AAV vector comprising a nucleic acid sequence encoding the siRNA molecules of the present invention may be a human serotype AAV vector. Such human AAV vector may be derived from any known serotype, e.g., from any one of serotypes AAV1-AAV11. As non-limiting examples, AAV vectors may be vectors comprising an AAV1-derived genome in an AAV1-derived capsid; vectors comprising an AAV2-derived genome in an AAV2-derived genome; vectors comprising an AAV4-derived genome in an AAV4 derived capsid; vectors comprising an AAV6-derived genome in an AAV6 derived capsid or vectors comprising an AAV9-derived genome in an AAV9 derived capsid.

In other embodiments, the AAV vector comprising a nucleic acid sequence for encoding siRNA molecules of the present invention may be a pseudotyped hybrid or chimeric AAV vector which contains sequences and/or components originating from at least two different AAV serotypes. Pseudotyped AAV vectors may be vectors comprising an AAV genome derived from one AAV serotype and a capsid protein derived at least in part from a different AAV serotype. As non-limiting examples, such pseudotyped AAV vectors may be vectors comprising an AAV2-derived genome in an AAV1-derived capsid; or vectors comprising an AAV2-derived genome in an AAV6-derived capsid; or vectors comprising an AAV2-derived genome in an AAV4-derived capsid; or an AAV2-derived genome in an AAV9-derived capsid. In like fashion, the present invention contemplates any hybrid or chimeric AAV vector.

In other embodiments, AAV vectors comprising a nucleic acid sequence encoding the siRNA molecules of the present invention may be used to deliver siRNA molecules to the central nervous system (e.g., U.S. Pat. No. 6,180,613; the contents of which is herein incorporated by reference in its entirety).

In some aspects, the AAV vectors comprising a nucleic acid sequence encoding the siRNA molecules of the present invention may further comprise a modified capsid including peptides from non-viral origin. In other aspects, the AAV vector may contain a CNS specific chimeric capsid to facilitate the delivery of encoded siRNA duplexes into the brain and the spinal cord. For example, an alignment of cap nucleotide sequences from AAV variants exhibiting CNS tropism may be constructed to identify variable region (VR) sequence and structure.

In one embodiment, the AAV vector comprising a nucleic acid sequence encoding the siRNA molecules of the present invention may encode siRNA molecules which are polycistronic molecules. The siRNA molecules may additionally comprise one or more linkers between regions of the siRNA molecules.

In one embodiment, the encoded siRNA molecule may be located downstream of a promoter in an expression vector such as, but not limited to, CMV, U6, CBA or a CBA promoter with a SV40 intron. Further, the encoded siRNA molecule may also be located upstream of the polyadenylation sequence in an expression vector. As a non-limiting example, the encoded siRNA molecule may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides downstream from the promoter and/or upstream of the polyadenylation sequence in an expression vector. As another non-limiting example, the encoded siRNA molecule may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 nucleotides downstream from the promoter and/or upstream of the polyadenylation sequence in an expression vector. As a non-limiting example, the encoded siRNA molecule may be located within the first 1%, 2%, 3%, 4%, 5%, 6%, 7%, 8%, 9%, 10%, 15%, 20%, 25% or more than 25% of the nucleotides downstream from the promoter and/or upstream of the polyadenylation sequence in an expression vector. As another non-limiting example, the encoded siRNA molecule may be located with the first 1-5%, 1-10%, 1-15%, 1-20%, 1-25%, 5-10%, 5-15%, 5-20%, 5-25%, 10-15%, 10-20%, 10-25%, 15-20%, 15-25%, or 20-25% downstream from the promoter and/or upstream of the polyadenylation sequence in an expression vector.

In one embodiment, the encoded siRNA molecule may be located upstream of the polyadenylation sequence in an expression vector. Further, the encoded siRNA molecule may be located downstream of a promoter such as, but not limited to, CMV, U6, CBA or a CBA promoter with a SV40 intron in an expression vector. As a non-limiting example, the encoded siRNA molecule may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides downstream from the promoter and/or upstream of the polyadenylation sequence in an expression vector. As another non-limiting example, the encoded siRNA molecule may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 nucleotides downstream from the promoter and/or upstream of the polyadenylation sequence in an expression vector. As a non-limiting example, the encoded siRNA molecule may be located within the first 1%, 2%, 3%, 4%, 5%, 6%, 7%, 8%, 9%, 10%, 15%, 20%, 25% or more than 25% of the nucleotides downstream from the promoter and/or upstream of the polyadenylation sequence in an expression vector. As another non-limiting example, the encoded siRNA molecule may be located with the first 1-5%, 1-10%, 1-15%, 1-20%, 1-25%, 5-10%, 5-15%, 5-20%, 5-25%, 10-15%, 10-20%, 10-25%, 15-20%, 15-25%, or 20-25% downstream from the promoter and/or upstream of the polyadenylation sequence in an expression vector.

In one embodiment, the encoded siRNA molecule may be located in a scAAV.

In one embodiment, the encoded siRNA molecule may be located in an ssAAV.

In one embodiment, the encoded siRNA molecule may be located near the 5′ end of the flip ITR in an expression vector. In another embodiment, the encoded siRNA molecule may be located near the 3′ end of the flip ITR in an expression vector. In yet another embodiment, the encoded siRNA molecule may be located near the 5′ end of the flop ITR in an expression vector. In yet another embodiment, the encoded siRNA molecule may be located near the 3′ end of the flop ITR in an expression vector. In one embodiment, the encoded siRNA molecule may be located between the 5′ end of the flip ITR and the 3′ end of the flop ITR in an expression vector. In one embodiment, the encoded siRNA molecule may be located between (e.g., half-way between the 5′ end of the flip ITR and 3′ end of the flop ITR or the 3′ end of the flop ITR and the 5′ end of the flip ITR), the 3′ end of the flip ITR and the 5′ end of the flip ITR in an expression vector. As a non-limiting example, the encoded siRNA molecule may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides downstream from the 5′ or 3′ end of an ITR (e.g., Flip or Flop ITR) in an expression vector. As a non-limiting example, the encoded siRNA molecule may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides upstream from the 5′ or 3′ end of an ITR (e.g., Flip or Flop ITR) in an expression vector. As another non-limiting example, the encoded siRNA molecule may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 nucleotides downstream from the 5′ or 3′ end of an ITR (e.g., Flip or Flop ITR) in an expression vector. As another non-limiting example, the encoded siRNA molecule may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 upstream from the 5′ or 3′ end of an ITR (e.g., Flip or Flop ITR) in an expression vector. As a non-limiting example, the encoded siRNA molecule may be located within the first 1%, 2%, 3%, 4%, 5%, 6%, 7%, 8%, 9%, 10%, 15%, 20%, 25% or more than 25% of the nucleotides upstream from the 5′ or 3′ end of an ITR (e.g., Flip or Flop ITR) in an expression vector. As another non-limiting example, the encoded siRNA molecule may be located with the first 1-5%, 1-10%, 1-15%, 1-20%, 1-25%, 5-10%, 5-15%, 5-20%, 5-25%, 10-15%, 10-20%, 10-25%, 15-20%, 15-25%, or 20-25% downstream from the 5′ or 3′ end of an ITR (e.g., Flip or Flop ITR) in an expression vector.

### Expression Vector

In one embodiment, an expression vector (e.g., AAV vector) may comprise at least one of the modulatory polynucleotides comprising at least one of the expression vectors described herein.

In one embodiment, an expression vector may comprise, from ITR to ITR recited 5′ to 3′, an ITR, a promoter, an intron, a modulatory polynucleotide, a polyA sequence and an ITR.

### Genome Size

In one embodiment, the vector genome which comprises a nucleic acid sequence encoding the modulatory polynucleotides described herein may be single stranded or double stranded vector genome. The size of the vector genome may be small, medium, large or the maximum size. Additionally, the vector genome may comprise a promoter and a polyA tail.

In one embodiment, the vector genome which comprises a nucleic acid sequence encoding the modulatory polynucleotides described herein may be a small single stranded vector genome. A small single stranded vector genome may be 2.7 to 3.5 kb in size such as about 2.7, 2.8, 2.9, 3.0, 3.1, 3.2, 3.3, 3.4, and 3.5 kb in size. As a non-limiting example, the small single stranded vector genome may be 3.2 kb in size. Additionally, the vector genome may comprise a promoter and a polyA tail.

In one embodiment, the vector genome which comprises a nucleic acid sequence encoding the modulatory polynucleotides described herein may be a small double stranded vector genome. A small double stranded vector genome may be 1.3 to 1.7 kb in size such as about 1.3, 1.4, 1.5, 1.6, and 1.7 kb in size. As a non-limiting example, the small double stranded vector genome may be 1.6 kb in size. Additionally, the vector genome may comprise a promoter and a polyA tail.

In one embodiment, the vector genome which comprises a nucleic acid sequence encoding the modulatory polynucleotides described herein e.g., siRNA or dsRNA, may be a medium single stranded vector genome. A medium single stranded vector genome may be 3.6 to 4.3 kb in size such as about 3.6, 3.7, 3.8, 3.9, 4.0, 4.1, 4.2 and 4.3 kb in size. As a non-limiting example, the medium single stranded vector genome may be 4.0 kb in size. Additionally, the vector genome may comprise a promoter and a polyA tail.

In one embodiment, the vector genome which comprises a nucleic acid sequence encoding the modulatory polynucleotides described herein may be a medium double stranded vector genome. A medium double stranded vector genome may be 1.8 to 2.1 kb in size such as about 1.8, 1.9, 2.0, and 2.1 kb in size. As a non-limiting example, the medium double stranded vector genome may be 2.0 kb in size. Additionally, the vector genome may comprise a promoter and a polyA tail.

In one embodiment, the vector genome which comprises a nucleic acid sequence encoding the modulatory polynucleotides described herein may be a large single stranded vector genome. A large single stranded vector genome may be 4.4 to 6.0 kb in size such as about 4.4, 4.5, 4.6, 4.7, 4.8, 4.9, 5.0, 5.1, 5.2, 5.3, 5.4, 5.5, 5.6, 5.7, 5.8, 5.9 and 6.0 kb in size. As a non-limiting example, the large single stranded vector genome may be 4.7 kb in size. As another non-limiting example, the large single stranded vector genome may be 4.8 kb in size. As yet another non-limiting example, the large single stranded vector genome may be 6.0 kb in size. Additionally, the vector genome may comprise a promoter and a polyA tail.

In one embodiment, the vector genome which comprises a nucleic acid sequence encoding the modulatory polynucleotides described herein may be a large double stranded vector genome. A large double stranded vector genome may be 2.2 to 3.0 kb in size such as about 2.2, 2.3, 2.4, 2.5, 2.6, 2.7, 2.8, 2.9 and 3.0 kb in size. As a non-limiting example, the large double stranded vector genome may be 2.4 kb in size. Additionally, the vector genome may comprise a promoter and a polyA tail.

### Promoters

A person skilled in the art may recognize that a target cell may require a specific promoter including but not limited to a promoter that is species specific, inducible, tissue-specific, or cell cycle-specific Parr et al., Nat. Med. 3:1145-9 (1997); the contents of which are herein incorporated by reference in its entirety).

In one embodiment, the promoter is a promoter deemed to be efficient to drive the expression of the modulatory polynucleotide.

In one embodiment, the promoter is a promoter having a tropism for the cell being targeted.

In one embodiment, the promoter is a weak promoter which provides expression of a payload e.g., a modulatory polynucleotide, e.g., siRNA or dsRNA, for a period of time in targeted tissues such as, but not limited to, nervous system tissues. Expression may be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years or more than 10 years. Expression may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years or 5-10 years. As a non-limiting example, the promoter is a weak promoter for sustained expression of a payload in nervous tissues.

In one embodiment, the promoter may be a promoter which is less than 1 kb. The promoter may have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800. The promoter may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800.

In one embodiment, the promoter may be a combination of two or more components such as, but not limited to, CMV and CBA. Each component may have a length of 200, 210, 220, 230, 240, 250, 260, 270, 280, 290, 300, 310, 320, 330, 340, 350, 360, 370, 380, 381, 382, 383, 384, 385, 386, 387, 388, 389, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800. Each component may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800. As a non-limiting example, the promoter is a combination of a 382 nucleotide CMV-enhancer sequence and a 260 nucleotide CBA-promoter sequence.

In one embodiment, the vector genome comprises at least one element to enhance the target specificity and expression (See e.g., Powell et al. Viral Expression Cassette Elements to Enhance Transgene Target Specificity and Expression in Gene Therapy, 2015; the contents of which are herein incorporated by reference in its entirety). Non-limiting examples of elements to enhance the transgene target specificity and expression include promoters, endogenous miRNAs, post-transcriptional regulatory elements (PREs), polyadenylation (PolyA) signal sequences and upstream enhancers (USEs), CMV enhancers and introns.

In one embodiment, the vector genome comprises at least one element to enhance the target specificity and expression (See e.g., Powell et al. Viral Expression Cassette Elements to Enhance Transgene Target Specificity and Expression in Gene Therapy, 2015; the contents of which are herein incorporated by reference in its entirety) such as promoters.

Promoters for which promote expression in most tissues include, but are not limited to, human elongation factor 1α-subunit (EF1α), immediate-early cytomegalovirus (CMV), chicken β-actin (CBA) and its derivative CAG, the β glucuronidase (GUSB), or ubiquitin C (UBC). Tissue-specific expression elements can be used to restrict expression to certain cell types such as, but not limited to, nervous system promoters which can be used to restrict expression to neurons, astrocytes, or oligodendrocytes. Non-limiting example of tissue-specific expression elements for neurons include neuron-specific enolase (NSE), platelet-derived growth factor (PDGF), platelet-derived growth factor B-chain (PDGF-β), the synapsin (Syn), the methyl-CpG binding protein 2 (MeCP2), CaMKII, mGluR2, NFL, NFH, nß2, PPE, Enk and EAAT2 promoters. A non-limiting example of a tissue-specific expression elements for astrocytes include the glial fibrillary acidic protein (GFAP) and EAAT2 promoters. A non-limiting example of a tissue-specific expression element for oligodendrocytes include the myelin basic protein (MBP) promoter.

In one embodiment, the vector genome comprises a ubiquitous promoter. Non-limiting examples of ubiquitous promoters include CMV, CBA (including derivatives CAG, CBh, etc.), EF-1α, PGK, UBC, GUSB (hGBp), and UCOE (promoter of HNRPA2B1-CBX3). Yu et al. (Molecular Pain 2011, 7:63; the contents of which are herein incorporated by reference in its entirety) evaluated the expression of eGFP under the CAG, EFIa, PGK and UBC promoters in rat DRG cells and primary DRG cells using lentiviral vectors and found that UBC showed weaker expression than the other 3 promoters and there was only 10-12% glia expression seen for all promoters. Soderblom et al. (E. Neuro 2015; the contents of which are herein incorporated by reference in its entirety) the expression of eGFP in AAV8 with CMV and UBC promoters and AAV2 with the CMV promoter after injection in the motor cortex. Intranasal administration of a plasmid containing a UBC or EFIa promoter showed a sustained airway expression greater than the expression with the CMV promoter (See e.g., Gill et al., Gene Therapy 2001, Vol. 8, 1539-1546; the contents of which are herein incorporated by reference in its entirety). Husain et al. (Gene Therapy 2009; the contents of which are herein incorporated by reference in its entirety) evaluated a HBH construct with a hGUSB promoter, a HSV-1LAT promoter and a NSE promoter and found that the HBH construct showed weaker expression than NSE in mice brain. Passini and Wolfe (J. Virol. 2001, 12382-12392, the contents of which are herein incorporated by reference in its entirety) evaluated the long-term effects of the HBH vector following an intraventricular injection in neonatal mice and found that there was sustained expression for at least 1 year. Low expression in all brain regions was found by Xu et al. (Gene Therapy 2001, 8, 1323-1332; the contents of which are herein incorporated by reference in its entirety) when NF-L and NF-H promoters were used as compared to the CMV-lacZ, CMV-luc, EF, GFAP, hENK, nAChR, PPE, PPE+wpre, NSE (0.3 kb), NSE (1.8 kb) and NSE (1.8 kb+wpre). Xu et al. found that the promoter activity in descending order was NSE (1.8 kb), EF, NSE (0.3 kb), GFAP, CMV, hENK, PPE, NFL and NFH. NFL is a 650-nucleotide promoter and NFH is a 920-nucleotide promoter which are both absent in the liver but NFH is abundant in the sensory proprioceptive neurons, brain and spinal cord and NFH is present in the heart. Scn8a is a 470-nucleotide promoter which expresses throughout the DRG, spinal cord and brain with particularly high expression seen in the hippocampal neurons and cerebellar Purkinje cells, cortex, thalmus and hypothalamus (See e.g., Drews et al. 2007 and Raymond et al. 2004; the contents of each of which are herein incorporated by reference in their entireties).

In one embodiment, the vector genome comprises an UBC promoter. The UBC promoter may have a size of 300-350 nucleotides. As a non-limiting example, the UBC promoter is 332 nucleotides.

In one embodiment, the vector genome comprises a GUSB promoter. The GUSB promoter may have a size of 350-400 nucleotides. As a non-limiting example, the GUSB promoter is 378 nucleotides. As a non-limiting example, the construct may be AAV-promoter-CMV/globin intron-hFXN-RBG, where the AAV may be self-complementary and the AAV may be the DJ serotype.

In one embodiment, the vector genome comprises a NFL promoter. The NFL promoter may have a size of 600-700 nucleotides. As a non-limiting example, the NFL promoter is 650 nucleotides. As a non-limiting example, the construct may be AAV-promoter-CMV/globin intron-hFXN-RBG, where the AAV may be self-complementary and the AAV may be the DJ serotype.

In one embodiment, the vector genome comprises a NFH promoter. The NFH promoter may have a size of 900-950 nucleotides. As a non-limiting example, the NFH promoter is 920 nucleotides. As a non-limiting example, the construct may be AAV-promoter-CMV/globin intron-hFXN-RBG, where the AAV may be self-complementary and the AAV may be the DJ serotype.

In one embodiment, the vector genome comprises a scn8a promoter. The scn8a promoter may have a size of 450-500 nucleotides. As a non-limiting example, the scn8a promoter is 470 nucleotides. As a non-limiting example, the construct may be AAV-promoter-CMV/globin intron-hFXN-RBG, where the AAV may be self-complementary and the AAV may be the DJ serotype.

In one embodiment, the vector genome comprises a FXN promoter.

In one embodiment, the vector genome comprises a PGK promoter.

In one embodiment, the vector genome comprises a CBA promoter.

In one embodiment, the vector genome comprises a CMV promoter.

In one embodiment, the vector genome comprises a liver or a skeletal muscle promoter. Non-limiting examples of liver promoters include hAAT and TBG. Non-limiting examples of skeletal muscle promoters include Desmin, MCK and C5-12.

In one embodiment, the AAV vector comprises an enhancer element, a promoter and/or a 5′UTR intron. The enhancer may be, but is not limited to, a CMV enhancer, the promoter may be, but is not limited to, a CMV, CBA, UBC, GUSB, NSE, Sunapsin, MeCP2, and GFAP promoter and the 5′UTR/intron may be, but is not limited to, SV40, and CBA-MVM. As a non-limiting example, the enhancer, promoter and/or intron used in combination may be: (1) CMV enhancer, CMV promoter, SV40 5′UTR intron; (2) CMV enhancer, CBA promoter, SV 40 5′UTR intron; (3) CMV enhancer, CBA promoter, CBA-MVM 5′UTR intron; (4) UBC promoter; (5) GUSB promoter; (6) NSE promoter; (7) Synapsin promoter; (8) MeCP2 promoter and (9) GFAP promoter.

In one embodiment, the AAV vector has an engineered promoter.

### Introns

In one embodiment, the vector genome comprises at least one element to enhance the transgene target specificity and expression (See e.g., Powell et al. Viral Expression Cassette Elements to Enhance Transgene Target Specificity and Expression in Gene Therapy, 2015; the contents of which are herein incorporated by reference in its entirety) such as an intron. Non-limiting examples of introns include, MVM (67-97 bps), F.IX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps).

In one embodiment, the intron may be 100-500 nucleotides in length. The intron may have a length of 80, 90, 100, 110, 120, 130, 140, 150, 160, 170, 171, 172, 173, 174, 175, 176, 177, 178, 179, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500. The promoter may have a length between 80-100, 80-120, 80-140, 80-160, 80-180, 80-200, 80-250, 80-300, 80-350, 80-400, 80-450, 80-500, 200-300, 200-400, 200-500, 300-400, 300-500, or 400-500.

In one embodiment, the AAV vector genome may comprise a promoter such as, but not limited to, CMV or U6. As a non-limiting example, the promoter for the AAV comprising the nucleic acid sequence for the siRNA molecules of the present invention is a CMV promoter. As another non-limiting example, the promoter for the AAV comprising the nucleic acid sequence for the siRNA molecules of the present invention is a U6 promoter.

In one embodiment, the AAV vector may comprise a CMV and a U6 promoter.

In one embodiment, the AAV vector may comprise a CBA promoter.

Introduction into Cells-Synthetic dsRNA

To ensure the chemical and biological stability of siRNA molecules (e.g., siRNA duplexes and dsRNA), it is important to deliver siRNA molecules inside the target cells. In some embodiments, the cells may include, but are not limited to, cells of mammalian origin, cells of human origins, embryonic stem cells, induced pluripotent stem cells, neural stem cells, and neural progenitor cells.

Nucleic acids, including siRNA, carry a net negative charge on the sugar-phosphate backbone under normal physiological conditions. In order to enter the cell, a siRNA molecule must come into contact with a lipid bilayer of the cell membrane, whose head groups are also negatively charged.

The siRNA duplexes can be complexed with a carrier that allows them to traverse cell membranes such as package particles to facilitate cellular uptake of the siRNA. The package particles may include, but are not limited to, liposomes, nanoparticles, cationic lipids, polyethylenimine derivatives, dendrimers, carbon nanotubes and the combination of carbon-made nanoparticles with dendrimers. Lipids may be cationic lipids and/or neutral lipids. In addition to well established lipophilic complexes between siRNA molecules and cationic carriers, siRNA molecules can be conjugated to a hydrophobic moiety, such as cholesterol (e.g., U.S. Patent Publication No. 20110110937; the content of which is herein incorporated by reference in its entirety). This delivery method holds a potential of improving in vitro cellular uptake and in vivo pharmacological properties of siRNA molecules. The siRNA molecules of the present invention may also be conjugated to certain cationic cell-penetrating peptides (CPPs), such as MPG, transportan or penetratin covalently or non-covalently (e.g., U.S. Patent Publication No. 20110086425; the content of which is herein incorporated by reference in its entirety).

Introduction into Cells-AAV Vectors

The siRNA molecules (e.g., siRNA duplexes) of the present invention may be introduced into cells using any of a variety of approaches such as, but not limited to, viral vectors (e.g., AAV vectors). These viral vectors are engineered and optimized to facilitate the entry of siRNA molecule into cells that are not readily amendable to transfection. Also, some synthetic viral vectors possess an ability to integrate the shRNA into the cell genome, thereby leading to stable siRNA expression and long-term knockdown of a target gene. In this manner, viral vectors are engineered as vehicles for specific delivery while lacking the deleterious replication and/or integration features found in wild-type virus.

In some embodiments, the siRNA molecules of the present invention are introduced into a cell by contacting the cell with a composition comprising a lipophilic carrier and a vector, e.g., an AAV vector, comprising a nucleic acid sequence encoding the siRNA molecules of the present invention. In other embodiments, the siRNA molecule is introduced into a cell by transfecting or infecting the cell with a vector, e.g., an AAV vector, comprising nucleic acid sequences capable of producing the siRNA molecule when transcribed in the cell. In some embodiments, the siRNA molecule is introduced into a cell by injecting into the cell a vector, e.g., an AAV vector, comprising a nucleic acid sequence capable of producing the siRNA molecule when transcribed in the cell.

In some embodiments, prior to transfection, a vector, e.g., an AAV vector, comprising a nucleic acid sequence encoding the siRNA molecules of the present invention may be transfected into cells.

In other embodiments, the vectors, e.g., AAV vectors, comprising the nucleic acid sequence encoding the siRNA molecules of the present invention may be delivered into cells by electroporation (e.g., U.S. Patent Publication No. 20050014264; the content of which is herein incorporated by reference in its entirety).

Other methods for introducing vectors, e.g., AAV vectors, comprising the nucleic acid sequence for the siRNA molecules described herein may include photochemical internalization as described in U. S. Patent publication No. 20120264807; the content of which is herein incorporated by reference in its entirety.

In some embodiments, the formulations described herein may contain at least one vector, e.g., AAV vectors, comprising the nucleic acid sequence encoding the siRNA molecules described herein. In one embodiment, the siRNA molecules may target the SOD1 gene at one target site. In another embodiment, the formulation comprises a plurality of vectors, e.g., AAV vectors, each vector comprising a nucleic acid sequence encoding a siRNA molecule targeting the SOD1 gene at a different target site. The SOD1 may be targeted at 2, 3, 4, 5 or more than 5 sites.

In one embodiment, the vectors, e.g., AAV vectors, from any relevant species, such as, but not limited to, human, dog, mouse, rat or monkey may be introduced into cells.

In one embodiment, the vectors, e.g., AAV vectors, may be introduced into cells which are relevant to the disease to be treated. As a non-limiting example, the disease is ALS and the target cells are motor neurons and astrocytes.

In one embodiment, the vectors, e.g., AAV vectors, may be introduced into cells which have a high level of endogenous expression of the target sequence.

In another embodiment, the vectors, e.g., AAV vectors, may be introduced into cells which have a low level of endogenous expression of the target sequence.

In one embodiment, the cells may be those which have a high efficiency of AAV transduction.

### Pharmaceutical Compositions and Formulation

In addition to the pharmaceutical compositions (vectors, e.g., AAV vectors, comprising a nucleic acid sequence encoding the siRNA molecules), provided herein are pharmaceutical compositions which are suitable for administration to humans, it will be understood by the skilled artisan that such compositions are generally suitable for administration to any other animal, e.g., to non-human animals, e.g., non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

In some embodiments, compositions are administered to humans, human patients, or subjects. For the purposes of the present disclosure, the phrase “active ingredient” generally refers either to the synthetic siRNA duplexes, the vector, e.g., AAV vector, encoding the siRNA duplexes, or to the siRNA molecule delivered by a vector as described herein.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered.

The vectors e.g., AAV vectors, comprising the nucleic acid sequence encoding the siRNA molecules of the present invention can be formulated using one or more excipients to: (1) increase stability; (2) increase cell transfection or transduction; (3) permit the sustained or delayed release; or (4) alter the biodistribution (e.g., target the viral vector to specific tissues or cell types such as brain and motor neurons).

Formulations of the present invention can include, without limitation, saline, lipidoids, liposomes, lipid nanoparticles, polymers, lipoplexes, core-shell nanoparticles, peptides, proteins, cells transfected with viral vectors (e.g., for transplantation into a subject), nanoparticle mimics and combinations thereof. Further, the viral vectors of the present invention may be formulated using self-assembled nucleic acid nanoparticles.

Formulations of the pharmaceutical compositions described herein may be prepared by any method known or hereafter developed in the art of pharmacology. In general, such preparatory methods include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21^{st} Edition, A. R. Gennaro, Lippincott, Williams & Wilkins, Baltimore, MD, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

In some embodiments, the formulations may comprise at least one inactive ingredient. As used herein, the term “inactive ingredient” refers to one or more inactive agents included in formulations. In some embodiments, all, none, or some of the inactive ingredients which may be used in the formulations of the present invention may be approved by the US Food and Drug Administration (FDA).

Formulations of vectors comprising the nucleic acid sequence for the siRNA molecules of the present invention may include cations or anions. In one embodiment, the formulations include metal cations such as, but not limited to, Zn2+, Ca2+, Cu2+, Mg+ and combinations thereof.

As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977); the content of each of which is incorporated herein by reference in their entirety.

The term “pharmaceutically acceptable solvate,” as used herein, means a compound of the invention wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

According to the present invention, the vector, e.g., AAV vector, comprising the nucleic acid sequence for the siRNA molecules of the present invention may be formulated for CNS delivery. Agents that cross the brain blood barrier may be used. For example, some cell penetrating peptides that can target siRNA molecules to the brain blood barrier endothelium may be used to formulate the siRNA duplexes targeting the SOD1 gene (e.g., Mathupala, Expert Opin Ther Pat., 2009, 19, 137-140; the content of which is incorporated herein by reference in its entirety).

### Administration

The vector, e.g., AAV vector, comprising a nucleic acid sequence encoding the siRNA molecules of the present invention may be administered by any route which results in a therapeutically effective outcome. These include, but are not limited to enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal.

In specific embodiments, compositions of vector, e.g., AAV vector, comprising a nucleic acid sequence encoding the siRNA molecules of the present invention may be administered in a way which facilitates the vectors or siRNA molecule to enter the central nervous system and penetrate into motor neurons.

In some embodiments, the vector, e.g., AAV vector, comprising a nucleic acid sequence encoding the siRNA molecules of the present invention may be administered by muscular injection. Rizvanov et al. demonstrated for the first time that siRNA molecules, targeting mutant human SOD1 mRNA, is taken up by the sciatic nerve, retrogradely transported to the perikarya of motor neurons, and inhibits mutant SOD1 mRNA in SOD1^{G93A} transgenic ALS mice (Rizvanov A A et al., Exp. Brain Res., 2009, 195 (1), 1-4; the content of which is incorporated herein by reference in its entirety). Another study also demonstrated that muscle delivery of AAV expressing small hairpin RNAs (shRNAs) against the mutant SOD1 gene, led to significant mutant SOD1 knockdown in the muscle as well as innervating motor neurons (Towne C et al., Mol Ther., 2011; 19 (2): 274-283; the content of which is incorporated herein by reference in its entirety).

In some embodiments, AAV vectors that express siRNA duplexes of the present invention may be administered to a subject by peripheral injections and/or intranasal delivery. It was disclosed in the art that the peripheral administration of AAV vectors for siRNA duplexes can be transported to the central nervous system, for example, to the motor neurons (e.g., U.S. Patent Publication Nos. 20100240739; and 20100130594; the content of each of which is incorporated herein by reference in their entirety).

In other embodiments, compositions comprising at least one vector, e.g., AAV vector, comprising a nucleic acid sequence encoding the siRNA molecules of the present invention may be administered to a subject by intracranial delivery (See, e.g., U.S. Pat. No. 8,119,611; the content of which is incorporated herein by reference in its entirety).

The vector, e.g., AAV vector, comprising a nucleic acid sequence encoding the siRNA molecules of the present invention may be administered in any suitable form, either as a liquid solution or suspension, as a solid form suitable for liquid solution or suspension in a liquid solution. The siRNA duplexes may be formulated with any appropriate and pharmaceutically acceptable excipient.

The vector, e.g., AAV vector, comprising a nucleic acid sequence encoding the siRNA molecules of the present invention may be administered in a “therapeutically effective” amount, i.e., an amount that is sufficient to alleviate and/or prevent at least one symptom associated with the disease, or provide improvement in the condition of the subject.

In one embodiment, the vector, e.g., AAV vector, may be administered to the CNS in a therapeutically effective amount to improve function and/or survival for a subject with ALS. As a non-limiting example, the vector may be administered intrathecally.

In one embodiment, the vector, e.g., AAV vector, may be administered to a subject (e.g., to the CNS of a subject via intrathecal administration) in a therapeutically effective amount for the siRNA duplexes or dsRNA to target the motor neurons and astrocytes in the spinal cord and/or brain steam. As a non-limiting example, the siRNA duplexes or dsRNA may reduce the expression of SOD1 protein or mRNA. As another non-limiting example, the siRNA duplexes or dsRNA can suppress SOD1 and reduce SOD1 mediated toxicity. The reduction of SOD1 protein and/or mRNA as well as SOD1 mediated toxicity may be accomplished with almost no enhanced inflammation.

In one embodiment, the vector, e.g., AAV vector, may be administered to a subject (e.g., to the CNS of a subject) in a therapeutically effective amount to slow the functional decline of a subject (e.g., determined using a known evaluation method such as the ALS functional rating scale (ALSFRS)) and/or prolong ventilator-independent survival of subjects (e.g., decreased mortality or need for ventilation support). As a non-limiting example, the vector may be administered intrathecally.

In one embodiment, the vector, e.g., AAV vector, may be administered to the cisterna magna in a therapeutically effective amount to transduce spinal cord motor neurons and/or astrocytes. As a non-limiting example, the vector may be administered intrathecally.

In one embodiment, the vector, e.g., AAV vector, may be administered using intrathecal infusion in a therapeutically effective amount to transduce spinal cord motor neurons and/or astrocytes. As a non-limiting example, the vector may be administered intrathecally.

In one embodiment, the vector, e.g., AAV vector, comprising a modulatory polynucleotide may be formulated. As a non-limiting example the baricity and/or osmolality of the formulation may be optimized to ensure optimal drug distribution in the central nervous system or a region or component of the central nervous system.

In one embodiment, the vector, e.g., AAV vector, comprising a modulatory polynucleotide may be delivered to a subject via a single route administration.

In one embodiment, the vector, e.g., AAV vector, comprising a modulatory polynucleotide may be delivered to a subject via a multi-site route of administration. A subject may be administered the vector, e.g., AAV vector, comprising a modulatory polynucleotide at 2, 3, 4, 5 or more than 5 sites.

In one embodiment, a subject may be administered the vector, e.g., AAV vector, comprising a modulatory polynucleotide described herein using a bolus infusion.

In one embodiment, a subject may be administered the vector, e.g., AAV vector, comprising a modulatory polynucleotide described herein using sustained delivery over a period of minutes, hours, or days. The infusion rate may be changed depending on the subject, distribution, formulation, or another delivery parameter.

In one embodiment, the catheter may be located at more than one site in the spine for multi-site delivery. The vector, e.g., AAV vector, comprising a modulatory polynucleotide may be delivered in a continuous and/or bolus infusion. Each site of delivery may be a different dosing regimen or the same dosing regimen may be used for each site of delivery. As a non-limiting example, the sites of delivery may be in the cervical and the lumbar region. As another non-limiting example, the sites of delivery may be in the cervical region. As another non-limiting example, the sites of delivery may be in the lumbar region.

In one embodiment, a subject may be analyzed for spinal anatomy and pathology prior to delivery of the vector, e.g., AAV vector, comprising a modulatory polynucleotide described herein. As a non-limiting example, a subject with scoliosis may have a different dosing regimen and/or catheter location compared to a subject without scoliosis.

In one embodiment, the orientation of the spine of the subject during delivery of the vector, e.g., AAV vector, comprising a modulatory polynucleotide may be vertical to the ground.

In another embodiment, the orientation of the spine of the subject during delivery of the vector, e.g., AAV vector, comprising a modulatory polynucleotide may be horizontal to the ground.

In one embodiment, the spine of the subject may be at an angle as compared to the ground during the delivery of the vector, e.g., AAV vector, comprising a modulatory polynucleotide. The angle of the spine of the subject as compared to the ground may be at least 10, 20, 30, 40, 50, 60, 70, 80, 90, 100, 110, 120, 130, 140, 150 or 180 degrees.

In one embodiment, the delivery method and duration is chosen to provide broad transduction in the spinal cord. As a non-limiting example, intrathecal delivery is used to provide broad transduction along the rostral-caudal length of the spinal cord. As another non-limiting example, multi-site infusions provide a more uniform transduction along the rostral-caudal length of the spinal cord. As yet another non-limiting example, prolonged infusions provide a more uniform transduction along the rostral-caudal length of the spinal cord.

### Dosing

The pharmaceutical compositions of the present invention may be administered to a subject using any amount effective for reducing, preventing and/or treating a SOD1 associated disorder (e.g., ALS). The exact amount required will vary from subject to subject, depending on the species, age, and general condition of the subject, the severity of the disease, the particular composition, its mode of administration, its mode of activity, and the like.

The compositions of the present invention are typically formulated in unit dosage form for ease of administration and uniformity of dosage. It will be understood, however, that the total daily usage of the compositions of the present invention may be decided by the attending physician within the scope of sound medical judgment. The specific therapeutic effectiveness for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the siRNA duplexes employed; the duration of the treatment; drugs used in combination or coincidental with the specific compound employed; and like factors well known in the medical arts.

In one embodiment, the age and sex of a subject may be used to determine the dose of the compositions of the present invention. As a non-limiting example, a subject who is older may receive a larger dose (e.g., 5-10%, 10-20%, 15-30%, 20-50%, 25-50% or at least 1%, 2%, 3%, 4%, 5%, 10%, 20%, 30%, 40%, 50%, 60%, 70%, 80%, 90% or more than 90% more) of the composition as compared to a younger subject. As another non-limiting example, a subject who is younger may receive a larger dose (e.g., 5-10%, 10-20%, 15-30%, 20-50%, 25-50% or at least 1%, 2%, 3%, 4%, 5%, 10%, 20%, 30%, 40%, 50%, 60%, 70%, 80%, 90% or more than 90% more) of the composition as compared to an older subject. As yet another non-limiting example, a subject who is female may receive a larger dose (e.g., 5-10%, 10-20%, 15-30%, 20-50%, 25-50% or at least 1%, 2%, 3%, 4%, 5%, 10%, 20%, 30%, 40%, 50%, 60%, 70%, 80%, 90% or more than 90% more) of the composition as compared to a male subject. As yet another non-limiting example, a subject who is male may receive a larger dose (e.g., 5-10%, 10-20%, 15-30%, 20-50%, 25-50% or at least 1%, 2%, 3%, 4%, 5%, 10%, 20%, 30%, 40%, 50%, 60%, 70%, 80%, 90% or more than 90% more) of the composition as compared to a female subject

In some specific embodiments, the doses of AAV vectors for delivering siRNA duplexes of the present invention may be adapted dependent on the disease condition, the subject, and the treatment strategy.

In one embodiment, delivery of the compositions in accordance with the present invention to cells comprises a rate of delivery defined by [VG/hour=mL/hour*VG/mL] wherein VG is viral genomes, VG/mL is composition concentration, and mL/hour is rate of prolonged delivery.

In one embodiment, delivery of compositions in accordance with the present invention to cells may comprise a total concentration per subject between about 1×10⁶ VG and about 1×10¹⁶ VG. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 3×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹³, 2×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/subject.

In one embodiment, delivery of compositions in accordance with the present invention to cells may comprise a total concentration per subject between about 1×10⁶ VG/kg and about 1×10¹⁶ VG/kg. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 3×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 8.1×10¹², 8.2×10¹², 8.3×10¹², 8.4×10¹², 8.5×10¹², 8.6×10¹², 8.7×10¹², 8.8 ×10¹², 8.9×10¹², 9×10¹², 1×10¹³, 2×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/kg.

In one embodiment, about 105 to 10⁶ viral genome (unit) may be administered per dose.

In one embodiment, delivery of the compositions in accordance with the present invention to cells may comprise a total concentration between about 1×10⁶ VG/mL and about 1×10¹⁶ VG/mL. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 3.1×10¹², 3.2×10¹², 3.3×10¹², 3.4×10¹², 3.5×10¹², 3.6×10¹², 3.7×10¹², 3.8×10¹², 3.9×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 9×10¹², 1×10¹³, 2×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/mL.

In certain embodiments, the desired siRNA duple×dosage may be delivered using multiple administrations (e.g., two, three, four, five, si×, seven, eight, nine, ten, eleven, twelve, thirteen, fourteen, or more administrations). When multiple administrations are employed, split dosing regimens such as those described herein may be used. As used herein, a “split dose” is the division of single unit dose or total daily dose into two or more doses, e.g., two or more administrations of the single unit dose. As used herein, a “single unit dose” is a dose of any modulatory polynucleotide therapeutic administered in one dose/at one time/single route/single point of contact, i.e., single administration event. As used herein, a “total daily dose” is an amount given or prescribed in 24-hour period. It may be administered as a single unit dose. In one embodiment, the viral vectors comprising the modulatory polynucleotides of the present invention are administered to a subject in split doses. They may be formulated in buffer only or in a formulation described herein.

### Methods of Treatment of ALS

Provided in the present invention are methods for introducing the vectors, e.g., AAV vectors, comprising a nucleic acid sequence encoding the siRNA molecules of the present invention into cells, the method comprising introducing into said cells any of the vectors in an amount sufficient for degradation of target SOD1 mRNA to occur, thereby activating target-specific RNAi in the cells. In some aspects, the cells may be stem cells, neurons such as motor neurons, muscle cells and glial cells such as astrocytes.

Disclosed in the present invention are methods for treating ALS associated with abnormal SOD1function in a subject in need of treatment. The method optionally comprises administering to the subject a therapeutically effective amount of a composition comprising at least vectors, e.g., AAV vectors, comprising a nucleic acid sequence encoding the siRNA molecules of the present invention. As a non-limiting example, the siRNA molecules can silence SOD1 gene expression, inhibit SOD1 protein production, and reduce one or more symptoms of ALS in the subject such that ALS is therapeutically treated.

In some embodiments, the composition comprising the vectors, e.g., AAV vectors, comprising a nucleic acid sequence encoding the siRNA molecules of the present invention is administered to the central nervous system of the subject. In other embodiments, the composition comprising the vectors, e.g., AAV vectors, comprising a nucleic acid sequence encoding the siRNA molecules of the present invention is administered to the muscles of the subject

In particular, the vectors, e.g., AAV vectors, comprising a nucleic acid sequence encoding the siRNA molecules of the present invention may be delivered into specific types of targeted cells, including motor neurons; glial cells including oligodendrocyte, astrocyte, and microglia; and/or other cells surrounding neurons such as T cells. Studies in human ALS patients and animal SOD1 ALS models implicate glial cells as playing an early role in the dysfunction and death of motor neurons. Normal SOD1 in the surrounding, protective glial cells can prevent the motor neurons from dying even though mutant SOD1 is present in motor neurons (e.g., reviewed by Philips and Rothstein, Exp. Neurol., 2014 May 22. pii: S0014-4886 (14) 00157-5; the content of which is incorporated herein by reference in its entirety).

In some specific embodiments, the vectors, e.g., AAV vectors, comprising a nucleic acid sequence encoding the siRNA molecules of the present invention may be used as a therapy for ALS.

In some embodiments, the present composition is administered as a solo therapeutics or combination therapeutics for the treatment of ALS.

The vectors, e.g., AAV vectors, encoding siRNA duplexes targeting the SOD1 gene may be used in combination with one or more other therapeutic agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent.

Therapeutic agents that may be used in combination with the vectors, e.g., AAV vectors, encoding the nucleic acid sequence for the siRNA molecules of the present invention can be small molecule compounds which are antioxidants, anti-inflammatory agents, anti-apoptosis agents, calcium regulators, antiglutamatergic agents, structural protein inhibitors, and compounds involved in metal ion regulation.

Compounds tested for treating ALS which may be used in combination with the vectors described herein include, but are not limited to, antiglutamatergic agents: Riluzole, Topiramate, Talampanel, Lamotrigine, Dextromethorphan, Gabapentin and AMPA antagonist; Anti-apoptosis agents: Minocycline, Sodium phenylbutyrate and Arimoclomol; Anti-inflammatory agent: ganglioside, Celecoxib, Cyclosporine, Azathioprine, Cyclophosphamide, Plasmaphoresis, Glatiramer acetate and thalidomide; Ceftriaxone (Berry et al., Plos One, 2013, 8 (4)); Beat-lactam antibiotics; Pramipexole (a dopamine agonist) (Wang et al., Amyotrophic Lateral Scler., 2008, 9 (1), 50-58); Nimesulide in U.S. Patent Publication No. 20060074991; Diazoxide disclosed in U.S. Patent Publication No. 20130143873); pyrazolone derivatives disclosed in US Patent Publication No. 20080161378; free radical scavengers that inhibit oxidative stress-induced cell death, such as bromocriptine (US. Patent Publication No. 20110105517); phenyl carbamate compounds discussed in PCT Patent Publication No. 2013100571; neuroprotective compounds disclosed in U.S. Pat. Nos. 6,933,310 and 8,399,514 and US Patent Publication Nos. 20110237907 and 20140038927; and glycopeptides taught in U.S. Patent Publication No. 20070185012; the content of each of which is incorporated herein by reference in their entirety.

Therapeutic agents that may be used in combination therapy with the vectors, e.g., AAV vectors, encoding the nucleic acid sequence for the siRNA molecules of the present invention may be hormones or variants that can protect neuronal loss, such as adrenocorticotropic hormone (ACTH) or fragments thereof (e.g., U.S. Patent Publication No. 20130259875); Estrogen (e.g., U.S. Pat. Nos. 6,334,998 and 6,592,845); the content of each of which is incorporated herein by reference in their entirety.

Neurotrophic factors may be used in combination therapy with the vectors, e.g., AAV vectors, encoding the nucleic acid sequence for the siRNA molecules of the present invention for treating ALS. Generally, a neurotrophic factor is defined as a substance that promotes survival, growth, differentiation, proliferation and/or maturation of a neuron, or stimulates increased activity of a neuron. In some embodiments, the present methods further comprise delivery of one or more trophic factors into the subject in need of treatment. Trophic factors may include, but are not limited to, IGF-I, GDNF, BDNF, CTNF, VEGF, Colivelin, Xaliproden, Thyrotrophin-releasing hormone and ADNF, and variants thereof.

In one aspect, the vector, e.g., AAV vector, encoding the nucleic acid sequence for the at least one siRNA duplex targeting the SOD1 gene may be co-administered with AAV vectors expressing neurotrophic factors such as AAV-IGF-I (Vincent et al., Neuromolecular medicine, 2004, 6, 79-85; the content of which is incorporated herein by reference in its entirety) and AAV-GDNF (Wang et al., J Neurosci., 2002, 22, 6920-6928; the content of which is incorporated herein by reference in its entirety).

In some embodiments, the composition of the present invention for treating ALS is administered to the subject in need intravenously, intramuscularly, subcutaneously, intraperitoneally, intrathecally and/or intraventricularly, allowing the siRNA molecules or vectors comprising the siRNA molecules to pass through one or both the blood-brain barrier and the blood spinal cord barrier. In some aspects, the method includes administering (e.g., intraventricularly administering and/or intrathecally administering) directly to the central nervous system (CNS) of a subject (using, e.g., an infusion pump and/or a delivery scaffold) a therapeutically effective amount of a composition comprising vectors, e.g., AAV vectors, encoding the nucleic acid sequence for the siRNA molecules of the present invention. The vectors may be used to silence or suppress SOD1 gene expression, and/or reducing one or more symptoms of ALS in the subject such that ALS is therapeutically treated.

In certain aspects, the symptoms of ALS include, but are not limited to, motor neuron degeneration, muscle weakness, muscle atrophy, the stiffness of muscle, difficulty in breathing, slurred speech, fasciculation development, frontotemporal dementia and/or premature death are improved in the subject treated. In other aspects, the composition of the present invention is applied to one or both of the brain and the spinal cord. In other aspects, one or both of muscle coordination and muscle function are improved. In other aspects, the survival of the subject is prolonged.

In one embodiment, administration of the vectors, e.g., AAV vectors encoding a siRNA of the invention, to a subject may lower mutant SOD1 in the CNS of a subject. In another embodiment, administration of the vectors, e.g., AAV vectors, to a subject may lower wild-type SOD1 in the CNS of a subject. In yet another embodiment, administration of the vectors, e.g., AAV vectors, to a subject may lower both mutant SOD1 and wild-type SOD1 in the CNS of a subject. The mutant and/or wild-type SOD1 may be lowered by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in the CNS, a region of the CNS, or a specific cell of the CNS of a subject. As a non-limiting example, the vectors, e.g., AAV vectors may lower the expression of wild-type SOD1 by at least 50% in the motor neurons (e.g., ventral horn motor neurons) and/or astrocytes. As another non-limiting example, the vectors, e.g., AAV vectors may lower the expression of mutant SOD1 by at least 50% in the motor neurons (e.g., ventral horn motor neurons) and/or astrocytes. As yet another non-limiting example, the vectors, e.g., AAV vectors may lower the expression of wild-type SOD1 and mutant SOD1 by at least 50% in the motor neurons (e.g., ventral horn motor neurons) and/or astrocytes.

In one embodiment, administration of the vectors, e.g., AAV vectors, to a subject will reduce the expression of mutant and/or wild-type SOD1 in the spinal cord and the reduction of expression of the mutant and/or wild-type SOD1 will reduce the effects of ALS in a subject.

In one embodiment, the vectors, e.g., AAV vectors, may be administered to a subject who is in the early stages of ALS. Early-stage symptoms include, but are not limited to, muscles which are weak and soft or stiff, tight and spastic, cramping and twitching (fasciculations) of muscles, loss of muscle bulk (atrophy), fatigue, poor balance, slurred words, weak grip, and/or tripping when walking. The symptoms may be limited to a single body region or a mild symptom may affect more than one region. As a non-limiting example, administration of the vectors, e.g., AAV vectors, may reduce the severity and/or occurrence of the symptoms of ALS.

In one embodiment, the vectors, e.g., AAV vectors, may be administered to a subject who is in the middle stages of ALS. The middle stage of ALS includes, but is not limited to, more widespread muscle symptoms as compared to the early stage, some muscles are paralyzed while others are weakened or unaffected, continued muscle twitchings (fasciculations), unused muscles may cause contractures where the joints become rigid, painful and sometimes deformed, weakness in swallowing muscles may cause choking and greater difficulty eating and managing saliva, weakness in breathing muscles can cause respiratory insufficiency which can be prominent when lying down, and/or a subject may have bouts of uncontrolled and inappropriate laughing or crying (pseudobulbar affect). As a non-limiting example, administration of the vectors, e.g., AAV vectors, may reduce the severity and/or occurrence of the symptoms of ALS.

In one embodiment, the vectors, e.g., AAV vectors, may be administered to a subject who is in the late stages of ALS. The late stage of ALS includes, but is not limited to, voluntary muscles which are mostly paralyzed, the muscles that help move air in and out of the lungs are severely compromised, mobility is extremely limited, poor respiration may cause fatigue, fuzzy thinking, headaches and susceptibility to infection or diseases (e.g., pneumonia), speech is difficult and eating or drinking by mouth may not be possible.

In one embodiment, the vectors, e.g., AAV vectors, may be used to treat a subject with ALS who has a C9orf72 mutation.

In one embodiment, the vectors, e.g., AAV vectors, may be used to treat a subject with ALS who has TDP-43 mutations.

In one embodiment, the vectors, e.g., AAV vectors, may be used to treat a subject with ALS who has FUS mutations.

### Definitions

Unless stated otherwise, the following terms and phrases have the meanings described below. The definitions are not meant to be limiting in nature and serve to provide a clearer understanding of certain aspects of the present invention.

As used herein, the term “nucleic acid”, “polynucleotide” and ‘oligonucleotide” refer to any nucleic acid polymers composed of either polydeoxyribonucleotides (containing 2-deoxy-D-ribose), or polyribonucleotides (containing D-ribose), or any other type of polynucleotide which is an N glycoside of a purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single stranded RNA.

As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

As used herein, the term “RNA interfering” or “RNAi” refers to a sequence specific regulatory mechanism mediated by RNA molecules which results in the inhibition or interfering or “silencing” of the expression of a corresponding protein-coding gene. RNAi has been observed in many types of organisms, including plants, animals and fungi. RNAi occurs in cells naturally to remove foreign RNAs (e.g., viral RNAs). Natural RNAi proceeds via fragments cleaved from free dsRNA which direct the degradative mechanism to other similar RNA sequences. RNAi is controlled by the RNA-induced silencing complex (RISC) and is initiated by short/small dsRNA molecules in cell cytoplasm, where they interact with the catalytic RISC component argonaute. The dsRNA molecules can be introduced into cells exogenously. Exogenous dsRNA initiates RNAi by activating the ribonuclease protein Dicer, which binds and cleaves dsRNAs to produce double-stranded fragments of 21-25 base pairs with a few unpaired overhang bases on each end. These short double stranded fragments are called small interfering RNAs (siRNAs).

As used herein, the terms “short interfering RNA,” “small interfering RNA” or “siRNA” refer to an RNA molecule (or RNA analog) comprising between about 5-60 nucleotides (or nucleotide analogs) which is capable of directing or mediating RNAi. Preferably, a siRNA molecule comprises between about 15-30 nucleotides or nucleotide analogs, such as between about 16-25 nucleotides (or nucleotide analogs), between about 18-23 nucleotides (or nucleotide analogs), between about 19-22 nucleotides (or nucleotide analogs) (e.g., 19, 20, 21 or 22 nucleotides or nucleotide analogs), between about 19-25 nucleotides (or nucleotide analogs), and between about 19-24 nucleotides (or nucleotide analogs). The term “short” siRNA refers to a siRNA comprising 5-23 nucleotides, preferably 21 nucleotides (or nucleotide analogs), for example, 19, 20, 21 or 22 nucleotides. The term “long” siRNA refers to a siRNA comprising 24-60 nucleotides, preferably about 24-25 nucleotides, for example, 23, 24, 25 or 26 nucleotides. Short siRNAs may, in some instances, include fewer than 19 nucleotides, e.g., 16, 17 or 18 nucleotides, or as few as 5 nucleotides, provided that the shorter siRNA retains the ability to mediate RNAi. Likewise, long siRNAs may, in some instances, include more than 26 nucleotides, e.g., 27, 28, 29, 30, 35, 40, 45, 50, 55, or even 60 nucleotides, provided that the longer siRNA retains the ability to mediate RNAi or translational repression absent further processing, e.g., enzymatic processing, to a short siRNA. siRNAs can be single stranded RNA molecules (ss-siRNAs) or double stranded RNA molecules (ds-siRNAs) comprising a sense strand and an antisense strand which hybridized to form a duplex structure called siRNA duplex.

As used herein, the term “the antisense strand” or “the first strand” or “the guide strand” of a siRNA molecule refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process.

As used herein, the term “the sense strand” or “the second strand” or “the passenger strand” of a siRNA molecule refers to a strand that is complementary to the antisense strand or first strand. The antisense and sense strands of a siRNA molecule are hybridized to form a duplex structure. As used herein, a “siRNA duplex” includes a siRNA strand having sufficient complementarity to a section of about 10-50 nucleotides of the mRNA of the gene targeted for silencing and a siRNA strand having sufficient complementarity to form a duplex with the siRNA strand.

As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present invention, the ability to substitute a T is implied, unless otherwise stated. Perfect complementarity or 100% complementarity refers to the situation in which each nucleotide unit of one polynucleotide strand can form hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form hydrogen bond with each other, the polynucleotide strands exhibit 10% complementarity. In the same example, if 18 base pairs on each strand can form hydrogen bonds with each other, the polynucleotide strands exhibit 90% complementarity.

As used herein, the term “substantially complementary” means that the siRNA has a sequence (e.g., in the antisense strand) which is sufficient to bind the desired target mRNA, and to trigger the RNA silencing of the target mRNA.

As used herein, “targeting” means the process of design and selection of nucleic acid sequence that will hybridize to a target nucleic acid and induce a desired effect.

The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides, and peptides are well known in the art.

As used herein, the term “mutation” refers to any changing of the structure of a gene, resulting in a variant (also called “mutant”) form that may be transmitted to subsequent generations. Mutations in a gene may be caused by the alternation of single base in DNA, or the deletion, insertion, or rearrangement of larger sections of genes or chromosomes.

As used herein, the term “vector” means any molecule or moiety which transports, transduces or otherwise acts as a carrier of a heterologous molecule such as the siRNA molecule of the invention. A “viral vector” is a vector which comprises one or more polynucleotide regions encoding or comprising a molecule of interest, e.g., a transgene, a polynucleotide encoding a polypeptide or multi-polypeptide or a modulatory nucleic acid such as small interfering RNA (siRNA). Viral vectors are commonly used to deliver genetic materials into cells. Viral vectors are often modified for specific applications. Types of viral vectors include retroviral vectors, lentiviral vectors, adenoviral vectors and adeno-associated viral vectors.

The term “adeno-associated virus” or “AAV” or “AAV vector” as used herein refers to any vector which comprises or derives from components of an adeno-associated vector and is suitable to infect mammalian cells, preferably human cells. The term AAV vector typically designates an AAV type viral particle or virion comprising a nucleic acid molecule encoding a siRNA duplex. The AAV vector may be derived from various serotypes, including combinations of serotypes (i.e., “pseudotyped” AAV) or from various genomes (e.g., single stranded or self-complementary). In addition, the AAV vector may be replication defective and/or targeted.

As used herein, the phrase “inhibit expression of a gene” means to cause a reduction in the amount of an expression product of the gene. The expression product can be a RNA molecule transcribed from the gene (e.g., an mRNA) or a polypeptide translated from an mRNA transcribed from the gene. Typically a reduction in the level of an mRNA results in a reduction in the level of a polypeptide translated therefrom. The level of expression may be determined using standard techniques for measuring mRNA or protein.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe or cell or tissue thereof).

As used herein, the term “modified” refers to a changed state or structure of a molecule of the invention. Molecules may be modified in many ways including chemically, structurally, and functionally.

As used herein, the term “synthetic” means produced, prepared, and/or manufactured by the hand of man. Synthesis of polynucleotides or polypeptides or other molecules of the present invention may be chemical or enzymatic.

As used herein, the term “transfection” refers to methods to introduce exogenous nucleic acids into a cell. Methods of transfection include, but are not limited to, chemical methods, physical treatments and cationic lipids or mixtures. The list of agents that can be transfected into a cell is large and includes, but is not limited to, siRNA, sense and/or anti-sense sequences, DNA encoding one or more genes and organized into an expression plasmid, proteins, protein fragments, and more.

As used herein, “off target” refers to any unintended effect on any one or more target, gene, or cellular transcript.

As used herein, the phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “effective amount” of an agent is that amount sufficient to effect beneficial or desired results, for example, clinical results, and, as such, an “effective amount” depends upon the context in which it is being applied. For example, in the context of administering an agent that treats ALS, an effective amount of an agent is, for example, an amount sufficient to achieve treatment, as defined herein, of ALS, as compared to the response obtained without administration of the agent.

As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates such as chimpanzees and other apes and monkey species, and humans) and/or plants.

As used herein, the term “preventing” or “prevention” refers to delaying or forestalling the onset, development or progression of a condition or disease for a period of time, including weeks, months, or years.

The term “treatment” or “treating,” as used herein, refers to the application of one or more specific procedures used for the cure or amelioration of a disease. In certain embodiments, the specific procedure is the administration of one or more pharmaceutical agents. In the context of the present invention, the specific procedure is the administration of one or more siRNA duplexes or encoded dsRNA targeting SOD1 gene.

As used herein, the term “amelioration” or “ameliorating” refers to a lessening of severity of at least one indicator of a condition or disease. For example, in the context of neurodegeneration disorder, amelioration includes the reduction of neuron loss.

As used herein, the term “administering” refers to providing a pharmaceutical agent or composition to a subject.

As used herein, the term “neurodegeneration” refers to a pathologic state which results in neural cell death. A large number of neurological disorders share neurodegeneration as a common pathological state. For example, Alzheimer's disease, Parkinson's disease, Huntington's disease, and amyotrophic lateral sclerosis (ALS) all cause chronic neurodegeneration, which is characterized by a slow, progressive neural cell death over a period of several years, whereas acute neurodegeneration is characterized by a sudden onset of neural cell death as a result of ischemia, such as stroke, or trauma, such as traumatic brain injury, or as a result of axonal transection by demyelination or trauma caused, for example, by spinal cord injury or multiple sclerosis. In some neurological disorders, mainly one type of neuron cells are degenerative, for example, motor neuron degeneration in ALS.

### Equivalents and Scope

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

### EXAMPLES

### Example 1. SOD1 siRNA Design and Synthesis

SOD1 siRNA Design

siRNA design was carried out to identify siRNAs targeting human SOD1 gene. The design used the SOD1 transcripts for human ((Genebank access NO. NM_000454.4 (SEQ ID NO: 1)), cynomolgus ((Genebank access NO. XM_005548833.1) from the NCBI Refseq collection (release 63) (SEQ ID NO: 2)) and rhesus (SOD1 transcript ENSMMUT00000002415 (SEQ ID NO: 3) from the Ensembl project (release 75)) as described in Table 2.

TABLE 2
SOD1 gene sequences

| | | SEQ ID |
|---|---|---|
| SOD1 transcripts | Access No. | NO. |
| Human SOD1 cDNA (981 bp) | NM_000454.4 | 1 |
| cynomolgus SOD1 cDNA (465 bp) | XM_005548833.1 | 2 |
| rhesus SOD1 cDNA (464 bp) | ENSMMUT00000002415 | 3 |

The siRNA duplexes were designed to have 100% identity to the human SOD1 transcript for positions 2-18 of the antisense strand, and partial or 100% identity to the non-human primate SOD1 transcript for positions 2-18 of the antisense strand. In all siRNA duplexes, position 1 of the antisense strand was engineered to a U and position 19 of the sense strand was engineered to a C, in order to unpair the duplex at this position.

SOD1 siRNA Sequence Selection

Based on predicted selectivity of the antisense strand for human, cynomolgus and rhesus SOD1genes, and lack of match of the seed sequence at positions 2-7 of the antisense strand to human sequences in miRBase20.0, a total of 169 antisense and 169 sense human SOD1 derived oligonucleotides were synthesized and formed into duplexes (Table 3). The siRNA duplexes were then tested for in vitro inhibitory activity on endogenous SOD1 gene expression (SOD1 mRNA levels).

TABLE 3
Sense and antisense strand sequences of human SOD1 dsRNA

| | SIRNA | | sense strand | SEQ | | antisense strand | |
|---|---|---|---|---|---|---|---|
| | duplex | SS | sequence | ID | AS | sequence | SEQ |
| Start | ID | ID | (5′-3′) | NO | ID | (5′-3′) | ID NO |
| 26 | D-2741 | 7414 | CGGAGGUCUGGCCUA | 4 | 7415 | UUUAUAGGCCAGACCUCC | 173 |
| | | | UAACdTdT | | | GdTdT | |
| 27 | D-2742 | 7416 | GGAGGUCUGGCCUAU | 5 | 7417 | UUUUAUAGGCCAGACCUC | 174 |
| | | | AAACdTdT | | | CdTdT | |
| 28 | D-2743 | 7418 | GAGGUCUGGCCUAUA | 6 | 7419 | UCUUUAUAGGCCAGACCU | 175 |
| | | | AAGCdTdT | | | CdTdT | |
| 29 | D-2744 | 7420 | AGGUCUGGCCUAUAA | 7 | 7421 | UACUUUAUAGGCCAGACC | 176 |
| | | | AGUCdTdT | | | UdTdT | |
| 30 | D-2745 | 7422 | GGUCUGGCCUAUAAA | 8 | 7423 | UUACUUUAUAGGCCAGAC | 177 |
| | | | GUACdTdT | | | CdTdT | |
| 32 | D-2746 | 7424 | UCUGGCCUAUAAAGU | 9 | 7425 | UACUACUUUAUAGGCCAG | 178 |
| | | | AGUCdTdT | | | AdTdT | |
| 33 | D-2747 | 7426 | CUGGCCUAUAAAGUA | 10 | 7427 | UGACUACUUUAUAGGCCA | 179 |
| | | | GUCCdTdT | | | GdTdT | |
| 34 | D-2748 | 7428 | UGGCCUAUAAAGUAG | 11 | 7429 | UCGACUACUUUAUAGGCC | 180 |
| | | | UCGCdTdT | | | AdTdT | |
| 35 | D-2749 | 7430 | GGCCUAUAAAGUAGU | 12 | 7431 | UGCGACUACUUUAUAGGC | 181 |
| | | | CGCCdTdT | | | CdTdT | |
| 36 | D-2750 | 7432 | GCCUAUAAAGUAGUC | 13 | 7433 | UCGCGACUACUUUAUAGG | 182 |
| | | | GCGCdTdT | | | CdTdT | |
| 37 | D-2751 | 7434 | CCUAUAAAGUAGUCG | 14 | 7435 | UCCGCGACUACUUUAUAG | 183 |
| | | | CGGCdTdT | | | GdTdT | |
| 74 | D-2752 | 7436 | GUCGUAGUCUCCUGC | 15 | 7437 | UGCUGCAGGAGACUACGA | 184 |
| | | | AGCCdTdT | | | CdTdT | |
| 76 | D-2753 | 7438 | CGUAGUCUCCUGCAG | 16 | 7439 | UACGCUGCAGGAGACUAC | 185 |
| | | | CGUCdTdT | | | GdTdT | |
| 77 | D-2754 | 7440 | GUAGUCUCCUGCAGC | 17 | 7441 | UGACGCUGCAGGAGACUA | 186 |
| | | | GUCCdTdT | | | CdTdT | |
| 78 | D-2755 | 7442 | UAGUCUCCUGCAGCG | 18 | 7443 | UAGACGCUGCAGGAGACU | 187 |
| | | | UCUCdTdT | | | AdTdT | |
| 149 | D-2756 | 7444 | AUGGCGACGAAGGCC | 19 | 7445 | UCACGGCCUUCGUCGCCA | 188 |
| | | | GUGCdTdT | | | UdTdT | |
| 153 | D-2757 | 7446 | CGACGAAGGCCGUGU | 20 | 7447 | UCGCACACGGCCUUCGUC | 189 |
| | | | GCGCdTdT | | | GdTdT | |
| 157 | D-2758 | 7448 | GAAGGCCGUGUGCGU | 21 | 7449 | UAGCACGCACACGGCCUU | 190 |
| | | | GCUCdTdT | | | CdTdT | |
| 160 | D-2759 | 7450 | GGCCGUGUGCGUGCU | 22 | 7451 | UUUCAGCACGCACACGGC | 191 |
| | | | GAACdTdT | | | CdTdT | |
| 177 | D-2760 | 7452 | AGGGCGACGGCCCAG | 23 | 7453 | UGCACUGGGCCGUCGCCC | 192 |
| | | | UGCCdTdT | | | UdTdT | |
| 192 | D-2761 | 7454 | UGCAGGGCAUCAUCA | 24 | 7455 | UAAUUGAUGAUGCCCUGC | 193 |
| | | | AUUCdTdT | | | AdTdT | |
| 193 | D-2762 | 7456 | GCAGGGCAUCAUCAA | 25 | 7457 | UAAAUUGAUGAUGCCCUG | 194 |
| | | | UUUCdTdT | | | CdTdT | |
| 195 | D-2763 | 7458 | AGGGCAUCAUCAAUU | 26 | 7459 | UCGAAAUUGAUGAUGCCC | 195 |
| | | | UCGCdTdT | | | UdTdT | |
| 196 | D-2764 | 7460 | GGGCAUCAUCAAUUU | 27 | 7461 | UUCGAAAUUGAUGAUGCC | 196 |
| | | | CGACdTdT | | | CdTdT | |
| 197 | D-2765 | 7462 | GGCAUCAUCAAUUUC | 28 | 7463 | UCUCGAAAUUGAUGAUGC | 197 |
| | | | GAGCdTdT | | | CdTdT | |
| 198 | D-2766 | 7464 | GCAUCAUCAAUUUCG | 29 | 7465 | UGCUCGAAAUUGAUGAUG | 198 |
| | | | AGCCdTdT | | | CdTdT | |
| 199 | D-2767 | 7466 | CAUCAUCAAUUUCGA | 30 | 7467 | UUGCUCGAAAUUGAUGAU | 199 |
| | | | GCACdTdT | | | GdTdT | |
| 206 | D-2768 | 7468 | AAUUUCGAGCAGAAG | 31 | 7469 | UUUCCUUCUGCUCGAAAU | 200 |
| | | | GAACdTAT | | | UdTdT | |
| 209 | D-2769 | 7470 | UUCGAGCAGAAGGAA | 32 | 7471 | UACUUUCCUUCUGCUCGA | 201 |
| | | | AGUCdTdT | | | AdTdT | |
| 210 | D-2770 | 7472 | UCGAGCAGAAGGAAA | 33 | 7473 | UUACUUUCCUUCUGCUCG | 202 |
| | | | GUACdTdT | | | AdTdT | |
| 239 | D-2771 | 7474 | AAGGUGUGGGGAAGC | 34 | 7475 | UAAUGCUUCCCCACACCU | 203 |
| | | | AUUCdTdT | | | UdTdT | |
| 241 | D-2772 | 7476 | GGUGUGGGGAAGCAU | 35 | 7477 | UUUAAUGCUUCCCCACAC | 204 |
| | | | UAACdTdT | | | CdTdT | |
| 261 | D-2773 | 7478 | GACUGACUGAAGGCC | 36 | 7479 | UGCAGGCCUUCAGUCAGU | 205 |
| | | | UGCCdTdT | | | CdTdT | |
| 263 | D-2774 | 7480 | CUGACUGAAGGCCUG | 37 | 7481 | UAUGCAGGCCUUCAGUCA | 206 |
| | | | CAUCdTdT | | | GdTdT | |
| 264 | D-2775 | 7482 | UGACUGAAGGCCUGC | 38 | 7483 | UCAUGCAGGCCUUCAGUC | 207 |
| | | | AUGCdTdT | | | AdTdT | |
| 268 | D-2776 | 7484 | UGAAGGCCUGCAUGG | 39 | 7485 | UAAUCCAUGCAGGCCUUC | 208 |
| | | | AUUCdTdT | | | AdTdT | |
| 269 | D-2777 | 7486 | GAAGGCCUGCAUGGA | 40 | 7487 | UGAAUCCAUGCAGGCCUU | 209 |
| | | | UUCCdTdT | | | CdTdT | |
| 276 | D-2778 | 7488 | UGCAUGGAUUCCAUG | 41 | 7489 | UGAACAUGGAAUCCAUGC | 210 |
| | | | UUCCdTdT | | | AdTdT | |
| 278 | D-2779 | 7490 | CAUGGAUUCCAUGUU | 42 | 7491 | UAUGAACAUGGAAUCCAU | 211 |
| | | | CAUCATdT | | | GdTdT | |
| 281 | D-2780 | 7492 | GGAUUCCAUGUUCAU | 43 | 7493 | UCUCAUGAACAUGGAAUC | 212 |
| | | | GAGCdTdT | | | CdTdT | |
| 284 | D-2781 | 7494 | UUCCAUGUUCAUGAG | 44 | 7495 | UAAACUCAUGAACAUGGA | 213 |
| | | | UUUCdTdT | | | AdTdT | |
| 290 | D-2782 | 7496 | GUUCAUGAGUUUGGA | 45 | 7497 | UAUCUCCAAACUCAUGAA | 214 |
| | | | GAUCdTdT | | | CdTdT | |
| 291 | D-2783 | 7498 | UUCAUGAGUUUGGAG | 46 | 7499 | UUAUCUCCAAACUCAUGA | 215 |
| | | | AUACdTdT | | | AdTdT | |
| 295 | D-2784 | 7500 | UGAGUUUGGAGAUAA | 47 | 7501 | UGUAUUAUCUCCAAACUC | 216 |
| | | | UACCdTdT | | | AdTdT | |
| 296 | D-2785 | 7502 | GAGUUUGGAGAUAAU | 48 | 7503 | UUGUAUUAUCUCCAAACU | 217 |
| | | | ACACdTdT | | | CdTdT | |
| 316 | D-2786 | 7504 | AGGCUGUACCAGUGC | 49 | 7505 | UCCUGCACUGGUACAGCC | 218 |
| | | | AGGCdTdT | | | UdTdT | |
| 317 | D-2787 | 7506 | GGCUGUACCAGUGCA | 50 | 7507 | UACCUGCACUGGUACAGC | 219 |
| | | | GGUCdTdT | | | CdTdT | |
| 329 | D-2788 | 7508 | GCAGGUCCUCACUUU | 51 | 7509 | UAUUAAAGUGAGGACCUG | 220 |
| | | | AAUCdTdT | | | CdTdT | |
| 330 | D-2789 | 7510 | CAGGUCCUCACUUUA | 52 | 7511 | UGAUUAAAGUGAGGACCU | 221 |
| | | | AUCCdTdT | | | GdTdT | |
| 337 | D-2790 | 7512 | UCACUUUAAUCCUCU | 53 | 7513 | UGAUAGAGGAUUAAAGUG | 222 |
| | | | AUCCdTdT | | | AdTdT | |
| 350 | D-2791 | 7514 | CUAUCCAGAAAACAC | 54 | 7515 | UACCGUGUUUUCUGGAUA | 223 |
| | | | GGUCdTdT | | | GdTdT | |
| 351 | D-2792 | 7516 | UAUCCAGAAAACACG | 55 | 7517 | UCACCGUGUUUUCUGGAU | 224 |
| | | | GUGCdTdT | | | AdTdT | |
| 352 | D-2793 | 7518 | AUCCAGAAAACACGG | 56 | 7519 | UCCACCGUGUUUUCUGGA | 225 |
| | | | UGGCdTdT | | | UdTdT | |
| 354 | D-2794 | 7520 | CCAGAAAACACGGUG | 57 | 7521 | UGCCCACCGUGUUUUCUG | 226 |
| | | | GGCCdTdT | | | GdTdT | |
| 357 | D-2795 | 7522 | GAAAACACGGUGGGC | 58 | 7523 | UUUGGCCCACCGUGUUUU | 227 |
| | | | CAACdTdT | | | CdTdT | |
| 358 | D-2796 | 7524 | AAAACACGGUGGGCC | 59 | 7525 | UUUUGGCCCACCGUGUUU | 228 |
| | | | AAACdTdT | | | UdTdT | |
| 364 | D-2797 | 7526 | CGGUGGGCCAAAGGA | 60 | 7527 | UUCAUCCUUUGGCCCACC | 229 |
| | | | UGACdTdT | | | GdTdT | |
| 375 | D-2798 | 7528 | AGGAUGAAGAGAGGC | 61 | 7529 | UCAUGCCUCUCUUCAUCC | 230 |
| | | | AUGCdTdT | | | UdTdT | |
| 378 | D-2799 | 7530 | AUGAAGAGAGGCAUG | 62 | 7531 | UCAACAUGCCUCUCUUCA | 231 |
| | | | UUGCdTdT | | | UdTdT | |
| 383 | D-2800 | 7532 | GAGAGGCAUGUUGGA | 63 | 7533 | UGUCUCCAACAUGCCUCU | 232 |
| | | | GACCdTdT | | | CdTdT | |
| 384 | D-2801 | 7534 | AGAGGCAUGUUGGAG | 64 | 7535 | UAGUCUCCAACAUGCCUC | 233 |
| | | | ACUCdTdT | | | UdTdT | |
| 390 | D-2802 | 7536 | AUGUUGGAGACUUGG | 65 | 7537 | UUGCCCAAGUCUCCAACA | 234 |
| | | | GCACdTdT | | | UdTdT | |
| 392 | D-2803 | 7538 | GUUGGAGACUUGGGC | 66 | 7539 | UAUUGCCCAAGUCUCCAA | 235 |
| | | | AAUCdTdT | | | CdTdT | |
| 395 | D-2804 | 7540 | GGAGACUUGGGCAAU | 67 | 7541 | UCACAUUGCCCAAGUCUC | 236 |
| | | | GUGCdTdT | | | CdTdT | |
| 404 | D-2805 | 7542 | GGCAAUGUGACUGCU | 68 | 7543 | UGUCAGCAGUCACAUUGC | 237 |
| | | | GACCdTdT | | | CdTdT | |
| 406 | D-2806 | 7544 | CAAUGUGACUGCUGA | 69 | 7545 | UUUGUCAGCAGUCACAUU | 238 |
| | | | CAACdTdT | | | GdTdT | |
| 417 | D-2807 | 7546 | CUGACAAAGAUGGUG | 70 | 7547 | UCCACACCAUCUUUGUCA | 239 |
| | | | UGGCdTdT | | | GdTdT | |
| 418 | D-2808 | 7548 | UGACAAAGAUGGUGU | 71 | 7549 | UGCCACACCAUCUUUGUC | 240 |
| | | | GGCCdTdT | | | AdTdT | |
| 469 | D-2809 | 7550 | CUCAGGAGACCAUUG | 72 | 7551 | UAUGCAAUGGUCUCCUGA | 241 |
| | | | CAUCATdT | | | GdTdT | |
| 470 | D-2810 | 7552 | UCAGGAGACCAUUGC | 73 | 7553 | UGAUGCAAUGGUCUCCUG | 242 |
| | | | AUCCdTdT | | | AdTdT | |
| 475 | D-2811 | 7554 | AGACCAUUGCAUCAU | 74 | 7555 | UCCAAUGAUGCAAUGGUC | 243 |
| | | | UGGCdTdT | | | UdTdT | |
| 476 | D-2812 | 7556 | GACCAUUGCAUCAUU | 75 | 7557 | UGCCAAUGAUGCAAUGGU | 244 |
| | | | GGCCdTdT | | | CdTdT | |
| 480 | D-2813 | 7558 | AUUGCAUCAUUGGCC | 76 | 7559 | UUGCGGCCAAUGAUGCAA | 245 |
| | | | GCACdTdT | | | UdTdT | |
| 487 | D-2814 | 7560 | CAUUGGCCGCACACU | 77 | 7561 | UACCAGUGUGCGGCCAAU | 246 |
| | | | GGUCdTdT | | | GdTdT | |
| 494 | D-2815 | 7562 | CGCACACUGGUGGUC | 78 | 7563 | UAUGGACCACCAGUGUGC | 247 |
| | | | CAUCATdT | | | GdTdT | |
| 496 | D-2816 | 7564 | CACACUGGUGGUCCA | 79 | 7565 | UUCAUGGACCACCAGUGU | 248 |
| | | | UGACdTdT | | | GdTdT | |
| 497 | D-2817 | 7566 | ACACUGGUGGUCCAU | 80 | 7567 | UUUCAUGGACCACCAGUG | 249 |
| | | | GAACdTdT | | | UdTdT | |
| 501 | D-2818 | 7568 | UGGUGGUCCAUGAAA | 81 | 7569 | UCUUUUUCAUGGACCACC | 250 |
| | | | AAGCdTdT | | | AdTdT | |
| 504 | D-2819 | 7570 | UGGUCCAUGAAAAAG | 82 | 7571 | UCUGCUUUUUCAUGGACC | 251 |
| | | | CAGCdTdT | | | AdTdT | |
| 515 | D-2820 | 7572 | AAAGCAGAUGACUUG | 83 | 7573 | UGCCCAAGUCAUCUGCUU | 252 |
| | | | GGCCdTdT | | | UdTdT | |
| 518 | D-2821 | 7574 | GCAGAUGACUUGGGC | 84 | 7575 | UUUUGCCCAAGUCAUCUG | 253 |
| | | | AAACdTdT | | | CdTdT | |
| 522 | D-2822 | 7576 | AUGACUUGGGCAAAG | 85 | 7577 | UCACCUUUGCCCAAGUCA | 254 |
| | | | GUGCdTdT | | | UdTdT | |
| 523 | D-2823 | 7578 | UGACUUGGGCAAAGG | 86 | 7579 | UCCACCUUUGCCCAAGUC | 255 |
| | | | UGGCdTdT | | | AdTdT | |
| 524 | D-2824 | 7580 | GACUUGGGCAAAGGU | 87 | 7581 | UUCCACCUUUGCCCAAGU | 256 |
| | | | GGACdTdT | | | CdTdT | |
| 552 | D-2825 | 7582 | GUACAAAGACAGGAA | 88 | 7583 | UCGUUUCCUGUCUUUGUA | 257 |
| | | | ACGCdTAT | | | CdTdT | |
| 554 | D-2826 | 7584 | ACAAAGACAGGAAAC | 89 | 7585 | UAGCGUUUCCUGUCUUUG | 258 |
| | | | GCUCdTdT | | | UdTdT | |
| 555 | D-2827 | 7586 | CAAAGACAGGAAACG | 90 | 7587 | UCAGCGUUUCCUGUCUUU | 259 |
| | | | CUGCdTdT | | | GdTdT | |
| 562 | D-2828 | 7588 | AGGAAACGCUGGAAG | 91 | 7589 | UCGACUUCCAGCGUUUCC | 260 |
| | | | UCGCdTdT | | | UdTdT | |
| 576 | D-2829 | 7590 | GUCGUUUGGCUUGUG | 92 | 7591 | UCACCACAAGCCAAACGA | 261 |
| | | | GUGCdTdT | | | CdTdT | |
| 577 | D-2830 | 7592 | UCGUUUGGCUUGUGG | 93 | 7593 | UACACCACAAGCCAAACG | 262 |
| | | | UGUCdTdT | | | AdTdT | |
| 578 | D-2831 | 7594 | CGUUUGGCUUGUGGU | 94 | 7595 | UUACACCACAAGCCAAAC | 263 |
| | | | GUACdTdT | | | GdTdT | |
| 579 | D-2832 | 7596 | GUUUGGCUUGUGGUG | 95 | 7597 | UUUACACCACAAGCCAAA | 264 |
| | | | UAACdTdT | | | CdTdT | |
| 581 | D-2833 | 7598 | UUGGCUUGUGGUGUA | 96 | 7599 | UAAUUACACCACAAGCCA | 265 |
| | | | AUUCdTdT | | | AdTdT | |
| 583 | D-2834 | 7600 | GGCUUGUGGUGUAAU | 97 | 7601 | UCCAAUUACACCACAAGC | 266 |
| | | | UGGCdTdT | | | CdTdT | |
| 584 | D-2835 | 7602 | GCUUGUGGUGUAAUU | 98 | 7603 | UCCCAAUUACACCACAAG | 267 |
| | | | GGGCdTdT | | | CdTdT | |
| 585 | D-2836 | 7604 | CUUGUGGUGUAAUUG | 99 | 7605 | UUCCCAAUUACACCACAA | 268 |
| | | | GGACdTdT | | | GdTdT | |
| 587 | D-2837 | 7606 | UGUGGUGUAAUUGGG | 100 | 7607 | UGAUCCCAAUUACACCAC | 269 |
| | | | AUCCdTdT | | | AdTdT | |
| 588 | D-2838 | 7608 | GUGGUGUAAUUGGGA | 101 | 7609 | UCGAUCCCAAUUACACCA | 270 |
| | | | UCGCdTdT | | | CdTdT | |
| 589 | D-2839 | 7610 | UGGUGUAAUUGGGAU | 102 | 7611 | UGCGAUCCCAAUUACACC | 271 |
| | | | CGCCdTdT | | | AdTdT | |
| 593 | D-2840 | 7612 | GUAAUUGGGAUCGCC | 103 | 7613 | UUUGGGCGAUCCCAAUUA | 272 |
| | | | CAACdTdT | | | CdTdT | |
| 594 | D-2841 | 7614 | UAAUUGGGAUCGCCC | 104 | 7615 | UAUUGGGCGAUCCCAAUU | 273 |
| | | | AAUCdTdT | | | AdTdT | |
| 595 | D-2842 | 7616 | AAUUGGGAUCGCCCA | 105 | 7617 | UUAUUGGGCGAUCCCAAU | 274 |
| | | | AUACdTdT | | | UdTdT | |
| 596 | D-2843 | 7618 | AUUGGGAUCGCCCAA | 106 | 7619 | UUUAUUGGGCGAUCCCAA | 275 |
| | | | UAACdTdT | | | UdTdT | |
| 597 | D-2844 | 7620 | UUGGGAUCGCCCAAU | 107 | 7621 | UUUUAUUGGGCGAUCCCA | 276 |
| | | | AAACdTAT | | | AdTdT | |
| 598 | D-2845 | 7622 | UGGGAUCGCCCAAUA | 108 | 7623 | UGUUUAUUGGGCGAUCCC | 277 |
| | | | AACCdTdT | | | AdTdT | |
| 599 | D-2846 | 7624 | GGGAUCGCCCAAUAA | 109 | 7625 | UUGUUUAUUGGGCGAUCC | 278 |
| | | | ACACdTdT | | | CdTdT | |
| 602 | D-2847 | 7626 | AUCGCCCAAUAAACA | 110 | 7627 | UGAAUGUUUAUUGGGCGA | 279 |
| | | | UUCCdTdT | | | UdTdT | |
| 607 | D-2848 | 7628 | CCAAUAAACAUUCCC | 111 | 7629 | UCAAGGGAAUGUUUAUUG | 280 |
| | | | UUGCdTdT | | | GdTdT | |
| 608 | D-2849 | 7630 | CAAUAAACAUUCCCU | 112 | 7631 | UCCAAGGGAAUGUUUAUU | 281 |
| | | | UGGCdTdT | | | GdTdT | |
| 609 | D-2850 | 7632 | AAUAAACAUUCCCUU | 113 | 7633 | UUCCAAGGGAAUGUUUAU | 282 |
| | | | GGACdTdT | | | UdTdT | |
| 610 | D-2851 | 7634 | AUAAACAUUCCCUUG | 114 | 7635 | UAUCCAAGGGAAUGUUUA | 283 |
| | | | GAUCdTdT | | | UdTdT | |
| 611 | D-2852 | 7636 | UAAACAUUCCCUUGG | 115 | 7637 | UCAUCCAAGGGAAUGUUU | 284 |
| | | | AUGCdTdT | | | AdTdT | |
| 612 | D-2853 | 7638 | AAACAUUCCCUUGGA | 116 | 7639 | UACAUCCAAGGGAAUGUU | 285 |
| | | | UGUCdTdT | | | UdTdT | |
| 613 | D-2854 | 7640 | AACAUUCCCUUGGAU | 117 | 7641 | UUACAUCCAAGGGAAUGU | 286 |
| | | | GUACdTdT | | | UdTdT | |
| 616 | D-2855 | 7642 | AUUCCCUUGGAUGUA | 118 | 7643 | UGACUACAUCCAAGGGAA | 287 |
| | | | GUCCdTdT | | | UdTdT | |
| 621 | D-2856 | 7644 | CUUGGAUGUAGUCUG | 119 | 7645 | UCCUCAGACUACAUCCAA | 288 |
| | | | AGGCdTdT | | | GdTdT | |
| 633 | D-2857 | 7646 | CUGAGGCCCCUUAAC | 120 | 7647 | UUGAGUUAAGGGGCCUCA | 289 |
| | | | UCACdTdT | | | GdTdT | |
| 635 | D-2858 | 7648 | GAGGCCCCUUAACUC | 121 | 7649 | UGAUGAGUUAAGGGGCCU | 290 |
| | | | AUCCdTdT | | | CdTdT | |
| 636 | D-2859 | 7650 | AGGCCCCUUAACUCA | 122 | 7651 | UAGAUGAGUUAAGGGGCC | 291 |
| | | | UCUCdTdT | | | UdTdT | |
| 639 | D-2860 | 7652 | CCCCUUAACUCAUCU | 123 | 7653 | UAACAGAUGAGUUAAGGG | 292 |
| | | | GUUCdTdT | | | GdTdT | |
| 640 | D-2861 | 7654 | CCCUUAACUCAUCUG | 124 | 7655 | UUAACAGAUGAGUUAAGG | 293 |
| | | | UUACdTdT | | | GdTdT | |
| 641 | D-2862 | 7656 | CCUUAACUCAUCUGU | 125 | 7657 | UAUAACAGAUGAGUUAAG | 294 |
| | | | UAUCdTdT | | | GdTdT | |
| 642 | D-2863 | 7658 | CUUAACUCAUCUGUU | 126 | 7659 | UGAUAACAGAUGAGUUAA | 295 |
| | | | AUCCdTdT | | | GdTdT | |
| 643 | D-2864 | 7660 | UUAACUCAUCUGUUA | 127 | 7661 | UGGAUAACAGAUGAGUUA | 296 |
| | | | UCCCdTdT | | | AdTdT | |
| 644 | D-2865 | 7662 | UAACUCAUCUGUUAU | 128 | 7663 | UAGGAUAACAGAUGAGUU | 297 |
| | | | CCUCdTdT | | | AdTdT | |
| 645 | D-2866 | 7664 | AACUCAUCUGUUAUC | 129 | 7665 | UCAGGAUAACAGAUGAGU | 298 |
| | | | CUGCdTdT | | | UdTdT | |
| 654 | D-2867 | 7666 | GUUAUCCUGCUAGCU | 130 | 7667 | UUACAGCUAGCAGGAUAA | 299 |
| | | | GUACdTdT | | | CdTdT | |
| 660 | D-2868 | 7668 | CUGCUAGCUGUAGAA | 131 | 7669 | UCAUUUCUACAGCUAGCA | 300 |
| | | | AUGCdTdT | | | GdTdT | |
| 661 | D-2869 | 7670 | UGCUAGCUGUAGAAA | 132 | 7671 | UACAUUUCUACAGCUAGC | 301 |
| | | | UGUCdTdT | | | AdTdT | |
| 666 | D-2870 | 7672 | GCUGUAGAAAUGUAU | 133 | 7673 | UAGGAUACAUUUCUACAG | 302 |
| | | | CCUCdTdT | | | CdTdT | |
| 667 | D-2871 | 7674 | CUGUAGAAAUGUAUC | 134 | 7675 | UCAGGAUACAUUUCUACA | 303 |
| | | | CUGCdTdT | | | GdTdT | |
| 668 | D-2872 | 7676 | UGUAGAAAUGUAUCC | 135 | 7677 | UUCAGGAUACAUUUCUAC | 304 |
| | | | UGACdTdT | | | AdTdT | |
| 669 | D-2873 | 7678 | GUAGAAAUGUAUCCU | 136 | 7679 | UAUCAGGAUACAUUUCUA | 305 |
| | | | GAUCdTdT | | | CdTdT | |
| 673 | D-2874 | 7680 | AAAUGUAUCCUGAUA | 137 | 7681 | UGUUUAUCAGGAUACAUU | 306 |
| | | | AACCdTdT | | | UdTdT | |
| 677 | D-2875 | 7682 | GUAUCCUGAUAAACA | 138 | 7683 | UUAAUGUUUAUCAGGAUA | 307 |
| | | | UUACdTdT | | | CdTdT | |
| 692 | D-2876 | 7684 | UUAAACACUGUAAUC | 139 | 7685 | UUAAGAUUACAGUGUUUA | 308 |
| | | | UUACdTdT | | | AdTdT | |
| 698 | D-2877 | 7686 | ACUGUAAUCUUAAAA | 140 | 7687 | UCACUUUUAAGAUUACAG | 309 |
| | | | GUGCdTdT | | | UdTdT | |
| 699 | D-2878 | 7688 | CUGUAAUCUUAAAAG | 141 | 7689 | UACACUUUUAAGAUUACA | 310 |
| | | | UGUCdTdT | | | GdTdT | |
| 700 | D-2879 | 7690 | UGUAAUCUUAAAAGU | 142 | 7691 | UUACACUUUUAAGAUUAC | 311 |
| | | | GUACdTdT | | | AdTdT | |
| 701 | D-2880 | 7692 | GUAAUCUUAAAAGUG | 143 | 7693 | UUUACACUUUUAAGAUUA | 312 |
| | | | UAACdTdT | | | CdTdT | |
| 706 | D-2881 | 7694 | CUUAAAAGUGUAAUU | 144 | 7695 | UCACAAUUACACUUUUAA | 313 |
| | | | GUGCdTdT | | | GdTdT | |
| 749 | D-2882 | 7696 | UACCUGUAGUGAGAA | 145 | 7697 | UAGUUUCUCACUACAGGU | 314 |
| | | | ACUCdTdT | | | AdTdT | |
| 770 | D-2883 | 7698 | UUAUGAUCACUUGGA | 146 | 7699 | UUCUUCCAAGUGAUCAUA | 315 |
| | | | AGACdTdT | | | AdTdT | |
| 772 | D-2884 | 7700 | AUGAUCACUUGGAAG | 147 | 7701 | UAAUCUUCCAAGUGAUCA | 316 |
| | | | AUUCdTdT | | | UdTdT | |
| 775 | D-2885 | 7702 | AUCACUUGGAAGAUU | 148 | 7703 | UACAAAUCUUCCAAGUGA | 317 |
| | | | UGUCdTdT | | | UdTdT | |
| 781 | D-2886 | 7704 | UGGAAGAUUUGUAUA | 149 | 7705 | UAACUAUACAAAUCUUCC | 318 |
| | | | GUUCdTdT | | | AdTdT | |
| 800 | D-2887 | 7706 | UAUAAAACUCAGUUA | 150 | 7707 | UUUUUAACUGAGUUUUAU | 319 |
| | | | AAACdTdT | | | AdTdT | |
| 804 | D-2888 | 7708 | AAACUCAGUUAAAAU | 151 | 7709 | UGACAUUUUAACUGAGUU | 320 |
| | | | GUCCdTdT | | | UdTdT | |
| 819 | D-2889 | 7710 | GUCUGUUUCAAUGAC | 152 | 7711 | UCAGGUCAUUGAAACAGA | 321 |
| | | | CUGCdTdT | | | CdTdT | |
| 829 | D-2890 | 7712 | AUGACCUGUAUUUUG | 153 | 7713 | UUGGCAAAAUACAGGUCA | 322 |
| | | | CCACdTdT | | | UdTdT | |
| 832 | D-2891 | 7714 | ACCUGUAUUUUGCCA | 154 | 7715 | UGUCUGGCAAAAUACAGG | 323 |
| | | | GACCdTdT | | | UdTdT | |
| 833 | D-2892 | 7716 | CCUGUAUUUUGCCAG | 155 | 7717 | UAGUCUGGCAAAAUACAG | 324 |
| | | | ACUCdTdT | | | GdTdT | |
| 851 | D-2893 | 7718 | UAAAUCACAGAUGGG | 156 | 7719 | UAUACCCAUCUGUGAUUU | 325 |
| | | | UAUCdTdT | | | AdTdT | |
| 854 | D-2894 | 7720 | AUCACAGAUGGGUAU | 157 | 7721 | UUUAAUACCCAUCUGUGA | 326 |
| | | | UAACdTdT | | | UdTdT | |
| 855 | D-2895 | 7722 | UCACAGAUGGGUAUU | 158 | 7723 | UUUUAAUACCCAUCUGUG | 327 |
| | | | AAACdTdT | | | AdTdT | |
| 857 | D-2896 | 7724 | ACAGAUGGGUAUUAA | 159 | 7725 | UAGUUUAAUACCCAUCUG | 328 |
| | | | ACUCdTdT | | | UdTdT | |
| 858 | D-2897 | 7726 | CAGAUGGGUAUUAAA | 160 | 7727 | UAAGUUUAAUACCCAUCU | 329 |
| | | | CUUCdTdT | | | GdTdT | |
| 859 | D-2898 | 7728 | AGAUGGGUAUUAAAC | 161 | 7729 | UCAAGUUUAAUACCCAUC | 330 |
| | | | UUGCdTdT | | | UdTdT | |
| 861 | D-2899 | 7730 | AUGGGUAUUAAACUU | 162 | 7731 | UGACAAGUUUAAUACCCA | 331 |
| | | | GUCCdTdT | | | UdTdT | |
| 869 | D-2900 | 7732 | UAAACUUGUCAGAAU | 163 | 7733 | UGAAAUUCUGACAAGUUU | 332 |
| | | | UUCCdTdT | | | AdTdT | |
| 891 | D-2901 | 7734 | UCAUUCAAGCCUGUG | 164 | 7735 | UAUUCACAGGCUUGAAUG | 333 |
| | | | AAUCdTdT | | | AdTdT | |
| 892 | D-2902 | 7736 | CAUUCAAGCCUGUGA | 165 | 7737 | UUAUUCACAGGCUUGAAU | 334 |
| | | | AUACdTdT | | | GdTdT | |
| 906 | D-2903 | 7738 | AAUAAAAACCCUGUA | 166 | 7739 | UCCAUACAGGGUUUUUAU | 335 |
| | | | UGGCdTdT | | | UdTdT | |
| 907 | D-2904 | 7740 | AUAAAAACCCUGUAU | 167 | 7741 | UGCCAUACAGGGUUUUUA | 336 |
| | | | GGCCdTdT | | | UdTdT | |
| 912 | D-2905 | 7742 | AACCCUGUAUGGCAC | 168 | 7743 | UUAAGUGCCAUACAGGGU | 337 |
| | | | UUACdTdT | | | UdTdT | |
| 913 | D-2906 | 7744 | ACCCUGUAUGGCACU | 169 | 7745 | UAUAAGUGCCAUACAGGG | 338 |
| | | | UAUCdTdT | | | UdTdT | |
| 934 | D-2907 | 7746 | GAGGCUAUUAAAAGA | 170 | 7747 | UGAUUCUUUUAAUAGCCU | 339 |
| | | | AUCCdTdT | | | CdTdT | |
| 944 | D-2908 | 7748 | AAAGAAUCCAAAUUC | 171 | 7749 | UUUUGAAUUUGGAUUCUU | 340 |
| | | | AAACdTdT | | | UdTdT | |
| 947 | D-2909 | 7750 | GAAUCCAAAUUCAAA | 172 | 7751 | UUAGUUUGAAUUUGGAUU | 341 |
| | | | CUACdTdT | | | CdTdT | |

SOD1 siRNA Synthesis

Oligoribonucleotides were assembled on an ABI 3900 synthesizer (Applied Biosystems) according to the phosphoramidite oligomerization chemistry. The solid support was polystyrene loaded with 2′-deoxy-thymidine (purchased from Glen Research, Sterling, Virginia, USA) to give a synthesis scale of 0.2 μmol. Ancillary synthesis reagents, DNA and RNA phosphoramidites were obtained from SAFC Proligo (Hamburg, Germany). Specifically, 5′-O-(4,4′-dimethoxytrityl)-3′-O-(2-cyanoethyl-N,N-diisopropyl) phosphoramidite monomers of uridine (U), thymidine (dT), 4-N-acetylcytidine (CAc), 6-N-benzoyladenosine (Abz) and 2-N-isobutyrlguanosine (GiBu) with 2′-O-t-butyldimethylsilyl were used to build the oligomers sequence. Coupling time for all phosphoramidites (70 mM in Acetonitrile) was 3 min employing 5-Ethylthio-1H-tetrazole (ETT) as activator (0.5 M in Acetonitrile). Sequences were synthesized with removal of the final dimetoxytrityl protecting group on the synthesizer (“DMT off” synthesis). Upon completion of the solid phase synthesis oligoribonucleotides were cleaved from the solid support and de-protected using a 1:1 (v/v) mixture of aqueous methylamine (40%) and methylamine in ethanol (33%). After 90 minutes at 45° C. the solution was diluted with N,N-Dimethyl formamide (DMF) and triethylamine trihydrofluoride (TEA.HF) was added. After incubation at 45° C. for 2 hours the oligoribonucleotides were precipitated with 1 M NaOAc and a mixture of acetone and ethanol 4:1 (v/v). The pellets were dissolved in 1 M aqueous NaCl solution and desalted by size exclusion chromatography. This was accomplished using an AKTA Purifier HPLC System (GE Healthcare, Freiburg, Germany) equipped with a HiTrap 5 mL column (GE Healthcare). Identity of the oligoribonucleotides was confirmed by MALDI mass spectrometry or ESI mass spectrometry. To generate siRNAs from RNA single strands, equimolar amounts of complementary sense and antisense strands were mixed and annealed in a 20 mM NaCl, 4 mM sodium phosphate pH 6.8 buffer. siRNAs were stored frozen until use.

### Example 2. In Vitro Screening of SOD1 siRNAs for Human SOD1 mRNA Suppression

Human SOD1 targeting siRNAs (described in Table 3) were assayed for inhibition of endogenous SOD1 expression in HeLa cells, using the bDNA (branched DNA) assay to quantify SOD1 mRNA. Results from two dose assays were used to select a subset of SOD1 dsRNA duplexes for dose response experiments in 4 types of cultured cells to calculate IC50's.

### Cell Culture and Transfection

HeLa cells were obtained from ATCC (ATCC in Partnership with LGC Standards, Wesel, Germany) and cultured in HAM's F-12 Medium (Biochrom GmbH, Berlin, Germany) supplemented to contain 10% fetal calf serum (Ultra-low IgG from GIBCO/Life Technologies) and 1% Pen/Strep (Biochrom GmbH, Berlin, Germany) at 37° C. in an atmosphere with 5% CO₂ in a humidified incubator.

For transfection with siRNA, HeLa cells were seeded at a density of 19,000-20,000 cells/well in 96-well plates. Transfection of siRNA was carried out with Lipofectamine 2000 (Invitrogen/Life Technologies) according to the manufacturer's instructions. For the two-dose screen, SOD1 siRNA concentrations of 1 nM or 0.1 nM were used. Dose response experiments were done with SOD1 siRNA concentrations of 10, 2.5, 0.6, 0.16, 0.039, 0.0098, 0.0024, 0.0006, 0.00015, and 0.000038 nM. Control wells were transfected with luciferase siRNA, Aha-1 siRNA, PLGF siRNA, or a control mix of unrelated siRNAs.

### Branched DNA Assays-QuantiGene 2.0

After a 24-hour incubation with siRNA, media was removed and cells were lysed in 150 μl Lysis Mixture (1 volume lysis mixture, 2 volumes nuclease-free water) then incubated at 53° C. for 60 minutes. 80 μl Working Probe Set SOD1 (gene target) and 90 μl Working Probe Set GAPDH (endogenous control) and 20 μl or 10 μl of cell-lysate were then added to the Capture Plates. Capture Plates were incubated at 55° C. (for SOD1) and 53° C. (for GAPDH) (approx. 16-20 hrs). The next day, the Capture Plates were washed 3 times with at least 300 μl of 1× Wash Buffer (nuclease-free water, Buffer Component 1 and Wash Buffer Component 2) (after the last wash, invert the plate and blot it against clean paper towels). 100 μl of pre-Amplifier Working Reagent was added to the SOD1 Capture Plates, which were sealed with aluminum foil and incubated for 1 hour at 55° C. Following a 1-hour incubation, the wash step was repeated, then 100 μl Amplifier Working Reagent was added to both SOD1 and GAPDH capture plates. After 1 hour of incubation at 55° C. (SOD1) or 53° C. (GAPDH), the wash and dry steps were repeated, and 100 μl Label Probe was added. Capture plates were incubated at 50° C. (SOD1) or 53° C. (GAPDH) for 1 hour. The plates were then washed with 1× Wash Buffer and dried, and then 100 μl Substrate was added to the Capture Plates. Luminescence was read using 1420 Luminescence Counter (WALLAC VICTOR Light, Perkin Elmer, Rodgau-Jügesheim, Germany) following 30 minutes incubation in the dark.

bDNA Data Analysis

For each SOD1 siRNA or control siRNA, four wells were transfected in parallel, and individual datapoints were collected from each well. For each well, the SOD1 mRNA level was normalized to the GAPDH mRNA level. The activity of a given SOD1 siRNA was expressed as percent SOD1 mRNA concentration (normalized to GAPDH mRNA) in treated cells, relative to the SOD1 mRNA concentration (normalized to GAPDH mRNA) averaged across control wells.

Table 4 provides the results from the in vitro HeLa screen where the SOD1 siRNAs, the sequences of which are given in Table 3, were tested at either 1 nM or 0.1 nM. The mean percentage of SOD1 mRNA (normalized to GAPDH mRNA) remaining in treated cells relative to controls, as well as the standard deviation, is shown in Table 4 for each SOD1 siRNA. A number of SOD1 siRNAs at 1 nM were effective at reducing SOD1 mRNA levels by more than 80% in HeLa cells. Furthermore, a number of SOD1 siRNAs at 0.1 nM were effective at reducing SOD1 mRNA levels by more than 80% in HeLa cells.

TABLE 4
Two dose results of in vitro screen of SOD1 siRNAs in
HeLa cells for SOD1 gene expression inhibiting activity

| | Remaining SOD1 | | Remaining SOD1 | |
|---|---|---|---|---|
| | mRNA [% of | | mRNA [% of | |
| siRNA | Control] 24 hr After | SD | Control] 24 hr After | SD |
| duplex ID | 1 nM SOD1 siRNA | [%] | 0.1 nM SOD1 siRNA | [%] |
| D-2741 | 87.2 | 2.7 | 70.6 | 3 |
| D-2742 | 86.9 | 4.3 | 79.5 | 8.5 |
| D-2743 | 89.6 | 3.6 | 80.6 | 8.8 |
| D-2744 | 83.8 | 7.2 | 75.9 | 8.5 |
| D-2745 | 95.1 | 9.1 | 84.1 | 6.8 |
| D-2746 | 111.3 | 3.6 | 92.0 | 7.2 |
| D-2747 | 100.0 | 6.1 | 92.9 | 4.4 |
| D-2748 | 100.4 | 3.1 | 91.6 | 12 |
| D-2749 | 87.1 | 2.9 | 96.4 | 13 |
| D-2750 | 94.2 | 7.1 | 93.1 | 8 |
| D-2751 | 85.4 | 7.2 | 96.1 | 8 |
| D-2752 | 27.2 | 3.6 | 70.2 | 6.5 |
| D-2753 | 25.5 | 4.8 | 67.5 | 4.5 |
| D-2754 | 23.2 | 4 | 70.2 | 2.3 |
| D-2755 | 36.6 | 3.7 | 75.5 | 11 |
| D-2756 | 9.1 | 0.7 | 29.2 | 2.6 |
| D-2757 | 3.9 | 0.6 | 9.0 | 1.8 |
| D-2758 | 6.4 | 1.1 | 13.9 | 2.8 |
| D-2759 | 6.7 | 1.1 | 14.1 | 1 |
| D-2760 | 32.3 | 3.4 | 61.9 | 8.8 |
| D-2761 | 12.9 | 3.6 | 41.7 | 8.3 |
| D-2762 | 16.9 | 2.6 | 41.2 | 10 |
| D-2763 | 5.7 | 1.3 | 10.5 | 3.4 |
| D-2764 | 9.2 | 2.7 | 19.5 | 4.9 |
| D-2765 | 13.6 | 1.9 | 29.4 | 8.8 |
| D-2766 | 8.7 | 1.1 | 28.1 | 6.6 |
| D-2767 | 10.4 | 1.6 | 24.7 | 5.9 |
| D-2768 | 13.0 | 1.4 | 27.7 | 7.3 |
| D-2769 | 25.3 | 1.9 | 57.4 | 7.5 |
| D-2770 | 14.9 | 1.6 | 35.5 | 4.4 |
| D-2771 | 11.4 | 1.8 | 32.6 | 8.6 |
| D-2772 | 10.6 | 1.3 | 27.9 | 4.7 |
| D-2773 | 14.3 | 1.4 | 35.7 | 3.1 |
| D-2774 | 7.1 | 1.3 | 23.0 | 1.5 |
| D-2775 | 9.8 | 0.9 | 31.3 | 3.3 |
| D-2776 | 11.1 | 2.9 | 31.3 | 5.3 |
| D-2777 | 47.8 | 5.5 | 80.9 | 4.6 |
| D-2778 | 7.4 | 0.6 | 26.5 | 4.2 |
| D-2779 | 7.9 | 0.6 | 17.9 | 3 |
| D-2780 | 12.5 | 1.3 | 31.7 | 5.6 |
| D-2781 | 16.3 | 2.3 | 39.1 | 8 |
| D-2782 | 10.2 | 3.1 | 25.4 | 3 |
| D-2783 | 13.5 | 3.5 | 33.4 | 6.5 |
| D-2784 | 12.3 | 2.5 | 36.3 | 5.4 |
| D-2785 | 14.6 | 3 | 30.5 | 7.4 |
| D-2786 | 16.2 | 3.5 | 42.6 | 8 |
| D-2787 | 14.4 | 4.2 | 37.3 | 6.5 |
| D-2788 | 9.8 | 3 | 21.6 | 6.6 |
| D-2789 | 18.5 | 5.9 | 48.9 | 12 |
| D-2790 | 11.6 | 3.8 | 28.1 | 5.6 |
| D-2791 | 8.9 | 1.8 | 26.6 | 5.6 |
| D-2792 | 8.1 | 1.4 | 25.6 | 5.3 |
| D-2793 | 9.3 | 1.6 | 26.6 | 3 |
| D-2794 | 8.9 | 1.9 | 25.8 | 4.2 |
| D-2795 | 22.6 | 3.4 | 59.5 | 9.9 |
| D-2796 | 15.1 | 0.7 | 43.0 | 1.9 |
| D-2797 | 21.1 | 2.5 | 43.0 | 1.3 |
| D-2798 | 10.4 | 1.2 | 28.0 | 5.1 |
| D-2799 | 11.0 | 1.2 | 29.8 | 3.3 |
| D-2800 | 21.3 | 2.4 | 52.4 | 4.7 |
| D-2801 | 12.3 | 3.3 | 28.7 | 4 |
| D-2802 | 8.4 | 1.8 | 18.8 | 3.7 |
| D-2803 | 5.9 | 1 | 12.1 | 4.1 |
| D-2804 | 11.8 | 1.6 | 28.9 | 7.5 |
| D-2805 | 13.5 | 2.6 | 34.5 | 7.5 |
| D-2806 | 5.5 | 1.1 | 10.4 | 2.5 |
| D-2807 | 8.5 | 1.3 | 24.2 | 6.6 |
| D-2808 | 9.5 | 1.5 | 26.0 | 1.4 |
| D-2809 | 7.5 | 0.9 | 17.7 | 2.8 |
| D-2810 | 12.1 | 2 | 43.1 | 8.3 |
| D-2811 | 5.6 | 0.8 | 16.7 | 7 |
| D-2812 | 14.2 | 1.4 | 42.5 | 8.2 |
| D-2813 | 29.0 | 3.4 | 66.7 | 13 |
| D-2814 | 35.7 | 3.5 | 73.4 | 15 |
| D-2815 | 30.3 | 1.9 | 74.3 | 12 |
| D-2816 | 14.6 | 2.1 | 47.2 | 5.1 |
| D-2817 | 27.5 | 1.8 | 70.5 | 6.6 |
| D-2818 | 9.6 | 0.8 | 32.9 | 7.2 |
| D-2819 | 9.0 | 0.8 | 29.1 | 3 |
| D-2820 | 10.8 | 1.4 | 38.7 | 3.5 |
| D-2821 | 5.8 | 0.4 | 19.4 | 6.1 |
| D-2822 | 10.5 | 2.5 | 46.3 | 6.8 |
| D-2823 | 3.5 | 1.1 | 18.8 | 3.5 |
| D-2824 | 9.9 | 3.2 | 43.8 | 0.8 |
| D-2825 | 6.6 | 2.6 | 29.7 | 1.1 |
| D-2826 | 8.0 | 1.9 | 40.6 | 7.2 |
| D-2827 | 7.0 | 1.2 | 25.2 | 4.5 |
| D-2828 | 6.4 | 2.2 | 22.4 | 1.7 |
| D-2829 | 14.8 | 2.7 | 45.5 | 7.4 |
| D-2830 | 9.4 | 2 | 28.5 | 6.5 |
| D-2831 | 8.6 | 2.8 | 28.4 | 6.6 |
| D-2832 | 12.3 | 3.2 | 43.4 | 3.2 |
| D-2833 | 20.5 | 5.2 | 66.7 | 9.1 |
| D-2834 | 10.7 | 2.5 | 35.9 | 2.2 |
| D-2835 | 11.6 | 2.4 | 37.7 | 4 |
| D-2836 | 24.1 | 3.3 | 57.0 | 4.2 |
| D-2837 | 98.7 | 12 | 96.7 | 4.3 |
| D-2838 | 20.5 | 4 | 49.5 | 1.4 |
| D-2839 | 10.0 | 2.4 | 31.9 | 4.3 |
| D-2840 | 50.2 | 8.3 | 89.2 | 7.4 |
| D-2841 | 70.8 | 11 | 87.1 | 7.9 |
| D-2842 | 79.7 | 21 | 90.9 | 3.6 |
| D-2843 | 24.2 | 1.2 | 57.2 | 8.4 |
| D-2844 | 21.5 | 6.4 | 51.4 | 1 |
| D-2845 | 12.9 | 2.2 | 39.4 | 7.3 |
| D-2846 | 10.2 | 2.6 | 30.5 | 2.6 |
| D-2847 | 40.5 | 9.7 | 70.0 | 6.5 |
| D-2848 | 41.8 | 7 | 63.7 | 6 |
| D-2849 | 24.7 | 6.8 | 51.3 | 8.1 |
| D-2850 | 79.4 | 7.5 | 76.5 | 16 |
| D-2851 | 28.1 | 6.5 | 72.0 | 8.8 |
| D-2852 | 13.8 | 2.1 | 56.9 | 4.8 |
| D-2853 | 32.1 | 9.5 | 72.2 | 12 |
| D-2854 | 21.5 | 3.9 | 58.8 | 10 |
| D-2855 | 39.8 | 10 | 75.4 | 5.5 |
| D-2856 | 14.4 | 3.4 | 40.4 | 5.8 |
| D-2857 | 8.6 | 1 | 18.4 | 4.5 |
| D-2858 | 10.1 | 1.1 | 19.1 | 4.8 |
| D-2859 | 10.9 | 1.3 | 20.9 | 5.4 |
| D-2860 | 7.4 | 1.3 | 11.7 | 3.8 |
| D-2861 | 5.0 | 1.4 | 12.6 | 2.6 |
| D-2862 | 5.5 | 1 | 13.8 | 2.7 |
| D-2863 | 8.2 | 1.3 | 26.5 | 4.3 |
| D-2864 | 9.1 | 1.6 | 40.2 | 3.4 |
| D-2865 | 6.3 | 0.6 | 22.8 | 3.4 |
| D-2866 | 7.0 | 1.7 | 17.8 | 4.3 |
| D-2867 | 9.3 | 0.8 | 31.7 | 6.2 |
| D-2868 | 10.3 | 2.5 | 30.8 | 6.5 |
| D-2869 | 9.4 | 4.3 | 34.7 | 4.6 |
| D-2870 | 5.9 | 0.6 | 18.1 | 2.6 |
| D-2871 | 6.5 | 1.1 | 13.5 | 1.5 |
| D-2872 | 10.5 | 1 | 31.3 | 5.3 |
| D-2873 | 7.0 | 1.1 | 20.8 | 3.7 |
| D-2874 | 9.4 | 2.4 | 35.3 | 5.7 |
| D-2875 | 5.4 | 1.1 | 13.5 | 2.4 |
| D-2876 | 14.1 | 4.6 | 45.9 | 5.2 |
| D-2877 | 64.5 | 9.8 | 64.0 | 9 |
| D-2878 | 57.0 | 14 | 62.9 | 8.1 |
| D-2879 | 71.4 | 12 | 79.4 | 8.6 |
| D-2880 | 79.7 | 11 | 100.9 | 4.9 |
| D-2881 | 72.8 | 12 | 82.8 | 5.6 |
| D-2882 | 64.4 | 8.8 | 73.2 | 6.9 |
| D-2883 | 80.1 | 4.9 | 86.3 | 13 |
| D-2884 | 69.6 | 5.8 | 74.2 | 13 |
| D-2885 | 76.9 | 2 | 76.7 | 18 |
| D-2886 | 74.0 | 0.7 | 80.4 | 3.4 |
| D-2887 | 77.7 | 8.7 | 88.6 | 16 |
| D-2888 | 70.3 | 5.1 | 66.2 | 2.2 |
| D-2889 | 71.2 | 3 | 67.3 | 7.3 |
| D-2890 | 75.3 | 7.9 | 71.2 | 6.4 |
| D-2891 | 74.6 | 8.4 | 72.4 | 4.3 |
| D-2892 | 72.5 | 6.9 | 71.6 | 5.7 |
| D-2893 | 73.9 | 3.8 | 83.7 | 2.9 |
| D-2894 | 66.9 | 5.7 | 72.4 | 4.9 |
| D-2895 | 71.6 | 8.9 | 72.1 | 9 |
| D-2896 | 71.0 | 5.6 | 74.4 | 1.3 |
| D-2897 | 74.4 | 7.9 | 78.0 | 3.8 |
| D-2898 | 74.0 | 5.8 | 73.5 | 1.6 |
| D-2899 | 71.0 | 10 | 74.1 | 9.7 |
| D-2900 | 71.3 | 4.1 | 77.8 | 5.8 |
| D-2901 | 64.8 | 9.4 | 82.0 | 11 |
| D-2902 | 53.6 | 5.2 | 82.7 | 15 |
| D-2903 | 66.8 | 2.6 | 101.1 | 13 |
| D-2904 | 62.6 | 7.8 | 87.5 | 20 |
| D-2905 | 67.1 | 14 | 74.0 | 4.1 |
| D-2906 | 64.0 | 3.2 | 73.9 | 12 |
| D-2907 | 66.4 | 7.3 | 82.0 | 11 |
| D-2908 | 72.6 | 20 | 85.2 | 23 |
| D-2909 | 80.0 | 7.3 | 77.2 | 12 |

Twelve of the most active SOD1 siRNAs at 0.1 nM in HeLa cells were evaluated in dose-response experiments. Table 5 provides the IC50 concentrations resulting in 50% SOD1 mRNA suppression relative to control for these twelve selected SOD1 siRNAs in HeLa cells. These twelve SOD1 siRNAs were particularly potent in this experimental paradigm, and exhibited IC50 values between 1 and 8 pM.

TABLE 5
IC50 results of in vitro assay of SOD1 siRNAs in HeLa
cells for SOD1 gene expression inhibiting activity

| siRNA duplex ID | IC50 Mean (pM) |
|---|---|
| D-2757 | 1 |
| D-2806 | 4 |
| D-2860 | 2 |
| D-2861 | 2 |
| D-2875 | 4 |
| D-2871 | 5 |
| D-2758 | 5 |
| D-2759 | 5 |
| D-2866 | 4 |
| D-2870 | 4 |
| D-2823 | 6 |
| D-2858 | 8 |

The dose response data from Hela cells used to identify the IC50s for these twelve SOD1 siRNAs are presented in detail below in Table 6. All twelve siRNAs were determined to have pM IC50s in Hela cells. The IC50 data for the SOD1 siRNAs in Table 5 are a summary of the data presented in Table 6 below.

TABLE 6
Dose response data for 12 SOD1 siRNAs in HeLa cells

| Remaining SOD1 mRNA (% of control) | | | | | | | | | | | |
|---|---|---|---|---|---|---|---|---|---|---|---|
| siRNA | | | | | | | | | | | |
| duplex | | | | | | | | | | | IC50 |
| ID | 10 nM | 2.5 nM | 0.6 nM | 0.16 nM | 0.039 nM | 0.0098 nM | 0.0024 nM | 0.0006 nM | 0.00015 nM | 0.000038 nM | (nM) |
| D-2757 | 2 | 2 | 2 | 3 | 6 | 16 | 33 | 57 | 77 | 86 | 0.001 |
| D-2806 | 2 | 3 | 3 | 6 | 13 | 32 | 59 | 83 | 90 | 105 | 0.004 |
| D-2860 | 5 | 5 | 5 | 6 | 10 | 22 | 50 | 68 | 87 | 92 | 0.002 |
| D-2861 | 4 | 4 | 4 | 5 | 10 | 25 | 51 | 73 | 81 | 92 | 0.002 |
| D-2875 | 4 | 4 | 4 | 7 | 15 | 34 | 62 | 78 | 82 | 92 | 0.004 |
| D-2871 | 4 | 5 | 4 | 8 | 18 | 43 | 62 | 78 | 87 | 90 | 0.005 |
| D-2758 | 5 | 5 | 5 | 9 | 17 | 41 | 70 | 81 | 97 | 111 | 0.005 |
| D-2759 | 4 | 4 | 4 | 7 | 15 | 35 | 63 | 82 | 87 | 94 | 0.005 |
| D-2866 | 3 | 3 | 4 | 8 | 17 | 39 | 54 | 79 | 80 | 76 | 0.004 |
| D-2870 | 4 | 5 | 5 | 8 | 18 | 41 | 59 | 77 | 93 | 101 | 0.004 |
| D-2823 | 3 | 3 | 4 | 7 | 20 | 42 | 65 | 81 | 86 | 92 | 0.006 |
| D-2858 | 5 | 5 | 5 | 9 | 21 | 46 | 72 | 82 | 88 | 94 | 0.008 |

### Example 3. In Vitro Screen of Selected SOD1 siRNAs Against Endogenous SOD1 mRNA Expression in SH-SY5Y Cells, U87 Cells and Primary Human Astrocytes

SH-SY5Y cells were obtained from ATCC (ATCC in Partnership with LGC Standards, Wesel, Germany) and cultured in Dulbecco's MEM (Biochrom GmbH, Berlin, Germany) supplemented to contain 15% FCS (Ultra-low IgG from GIBCO/Life Technologies), 1% L-Glutamine (Biochrom GmbH, Berlin, Germany) and 1% Pen/Strep (Biochrom GmbH, Berlin, Germany) at 37° C. in an atmosphere with 5% CO₂ in a humidified incubator.

U87MG cells were obtained from ATCC (ATCC in Partnership with LGC Standards, Wesel, Germany) and cultured in ATCC-formulated Eagle's Minimum Essential Medium (ATCC in Partnership with LGC Standards, Wesel, Germany) supplemented to contain 10% FCS (Ultra-low IgG from GIBCO/Life Technologies) and 1% Pen/Strep (Biochrom GmbH, Berlin, Germany) at 37° C. in an atmosphere with 5% CO₂ in a humidified incubator.

Primary human astrocytes were obtained from LONZA (Lonza Sales Ltd, Basel, Switzerland) and cultured in ABM Basal Medium (Lonza Sales Ltd, Basel, Switzerland) supplemented with AGM SingleQuot Kit (Lonza Sales Ltd, Basel, Switzerland) at 37° C. in an atmosphere with 5% CO₂ in a humidified incubator.

Transfection of SH-SY5Y cells, U87MG cells and primary human astrocytes with twelve selected siRNAs (D-2757, D-2806, D-2860, D-2861, D-2875, D-2871, D-2758, D-2759, D-2866, D-2870, D-2823, D-2858), and quantitation of SOD1 and GAPDH mRNA levels with bDNA were performed in a similar manner to that described for HeLa cells, except that the transfection reagents were Lipofectamine2000 (Invitrogen/Life Technologies) for SH-SY5Y cells, RNAiMAX (Invitrogen/Life Technologies) for U87 cells, and Lipofectamine2000 (Invitrogen/Life Technologies) for primary human astrocytes.

The dose response data from SH-SY5Y cells, U87MG cells and primary human astrocytes used to identify the IC50s for these twelve SOD1 siRNAs (D-2757, D-2806, D-2860, D-2861, D-2875, D-2871, D-2758, D-2759, D-2866, D-2870, D-2823, D-2858), are presented in detail below in Tables 7, 8 and 9, respectively. All twelve siRNAs were determined to have pM IC50s in U87 cells.

IC50 values are provided in Table 10. In primary human astrocytes, IC50s were higher than in SH-SY5Y and U87MG cells, in general.

TABLE 7
Dose response data for 12 SOD1 siRNAs in SH-SY5Y cells

| siRNA | | | | | | | | | | | |
|---|---|---|---|---|---|---|---|---|---|---|---|
| duplex | Remaining SOD1 mRNA (% of control) | | | | | | | | | | IC50 |
| ID | 10 nM | 2.5 nM | 0.6 nM | 0.16 nM | 0.039 nM | 0.0098 nM | 0.0024 nM | 0.0006 nM | 0.00015 nM | 0.000038 nM | (nM) |
| D-2757 | 8 | 13 | 16 | 22 | 36 | 55 | 72 | 92 | 107 | 114 | 0.013 |
| D-2806 | 11 | 12 | 15 | 26 | 40 | 71 | 103 | 121 | 117 | 131 | 0.025 |
| D-2860 | 11 | 15 | 17 | 26 | 42 | 63 | 79 | 86 | 92 | 96 | 0.022 |
| D-2861 | 12 | 14 | 16 | 19 | 37 | 60 | 82 | 83 | 87 | 94 | 0.017 |
| D-2875 | 20 | 25 | 35 | 59 | 79 | 92 | 96 | 95 | 99 | 104 | 0.234 |
| D-2871 | 15 | 19 | 23 | 42 | 71 | 87 | 95 | 94 | 99 | 96 | 0.103 |
| D-2758 | 24 | 35 | 36 | 58 | 91 | 96 | 134 | 123 | 105 | 94 | 0.369 |
| D-2759 | 10 | 11 | 16 | 25 | 43 | 67 | 85 | 94 | 104 | 108 | 0.026 |
| D-2866 | 17 | 19 | 24 | 42 | 72 | 93 | 93 | 102 | 103 | 101 | 0.105 |
| D-2870 | 19 | 22 | 26 | 40 | 62 | 88 | 100 | 105 | 105 | 105 | 0.078 |
| D-2823 | 11 | 16 | 25 | 47 | 64 | 84 | 91 | 98 | 105 | 95 | 0.099 |
| D-2858 | 16 | 21 | 25 | 46 | 68 | 91 | 92 | 95 | 103 | 116 | 0.106 |

TABLE 8
Dose response data for 12 SOD1 siRNAs in U87MG cells

| siRNA | | | | | | | | | | | |
|---|---|---|---|---|---|---|---|---|---|---|---|
| duplex | Remaining SOD1 mRNA (% of control) | | | | | | | | | | IC50 |
| ID | 10 nM | 2.5 nM | 0.6 nM | 0.16 nM | 0.039 nM | 0.0098 nM | 0.0024 nM | 0.0006 nM | 0.00015 nM | 0.000038 nM | (nM) |
| D-2757 | 3 | 4 | 3 | 4 | 5 | 8 | 19 | 50 | 86 | 99 | 0.001 |
| D-2806 | 4 | 3 | 3 | 3 | 4 | 8 | 18 | 49 | 81 | 106 | 0.001 |
| D-2860 | 4 | 4 | 5 | 5 | 6 | 8 | 20 | 46 | 72 | 93 | 0.001 |
| D-2861 | 5 | 6 | 6 | 6 | 8 | 15 | 39 | 67 | 87 | 93 | 0.001 |
| D-2875 | 4 | 5 | 5 | 5 | 6 | 9 | 19 | 45 | 76 | 99 | 0.001 |
| D-2871 | 5 | 5 | 5 | 5 | 6 | 11 | 24 | 50 | 77 | 86 | 0.001 |
| D-2758 | 7 | 9 | 6 | 7 | 10 | 25 | 64 | 99 | 103 | 112 | 0.004 |
| D-2759 | 6 | 6 | 5 | 6 | 8 | 21 | 50 | 80 | 93 | 104 | 0.002 |
| D-2866 | 4 | 4 | 4 | 5 | 8 | 17 | 38 | 64 | 86 | 94 | 0.001 |
| D-2870 | 5 | 5 | 5 | 5 | 7 | 7 | 13 | 31 | 63 | 85 | 0.003 |
| D-2823 | 4 | 4 | 4 | 4 | 6 | 13 | 34 | 61 | 74 | 94 | 0.001 |
| D-2858 | 7 | 6 | 6 | 7 | 8 | 14 | 33 | 54 | 71 | 94 | 0.001 |

TABLE 9
Dose response data for 12 SOD1 siRNAs in Primary Human Astrocytes

| siRNA | | | | | | | | | | | |
|---|---|---|---|---|---|---|---|---|---|---|---|
| duplex | Remaining SOD1 mRNA (% of control) | | | | | | | | | | IC50 |
| ID | 10 nM | 2.5 nM | 0.6 nM | 0.16 nM | 0.039 nM | 0.0098 nM | 0.0024 nM | 0.0006 nM | 0.00015 nM | 0.000038 nM | (nM) |
| D-2757 | 29 | 30 | 35 | 48 | 66 | 87 | 95 | 101 | 95 | 103 | 0.123 |
| D-2806 | 26 | 32 | 35 | 47 | 63 | 78 | 87 | 95 | 95 | 98 | 0.113 |
| D-2860 | 29 | 38 | 39 | 51 | 68 | 82 | 94 | 93 | 94 | 101 | 0.192 |
| D-2861 | 27 | 33 | 38 | 47 | 62 | 73 | 88 | 93 | 96 | 102 | 0.114 |
| D-2875 | 25 | 28 | 39 | 47 | 72 | 80 | 100 | 105 | 105 | 118 | 0.151 |
| D-2871 | 25 | 34 | 42 | 52 | 63 | 83 | 97 | 100 | 97 | 108 | 0.182 |
| D-2758 | 27 | 29 | 31 | 41 | 51 | 71 | 86 | 91 | 95 | 98 | 0.049 |
| D-2759 | 34 | 39 | 41 | 53 | 70 | 83 | 97 | 101 | 98 | 103 | 0.219 |
| D-2866 | 30 | 32 | 35 | 46 | 65 | 78 | 84 | 87 | 92 | 95 | 0.118 |
| D-2870 | 34 | 34 | 38 | 48 | 71 | 74 | 82 | 91 | 92 | 98 | 0.163 |
| D-2823 | 27 | 31 | 40 | 53 | 67 | 80 | 84 | 86 | 92 | 97 | 0.186 |
| D-2858 | 29 | 30 | 37 | 55 | 72 | 91 | 93 | 100 | 104 | 104 | 0.197 |

The IC50 data for SOD1 siRNAs in Table 10 is a summary of the data presented in Tables 7, 8 and 9.

TABLE 10
IC50 results of in vitro assays of SOD1 siRNAs in
SH-SY5Y cells, U87MG cells and primary human astrocytes
for SOD1 gene expression inhibiting activity

| | | | Primary Human |
|---|---|---|---|
| siRNA | SH-SY5Y IC50 | U87MG IC50 | Astrocytes IC 50 |
| duplex ID | Mean (pM) | Mean (pM) | Mean (pM) |
| D-2757 | 13 | 1 | 123 |
| D-2806 | 25 | 1 | 113 |
| D-2860 | 22 | 1 | 192 |
| D-2861 | 17 | 1 | 114 |
| D-2875 | 234 | 1 | 151 |
| D-2871 | 103 | 1 | 182 |
| D-2758 | 369 | 4 | 49 |
| D-2759 | 26 | 2 | 219 |
| D-2866 | 105 | 1 | 118 |
| D-2870 | 78 | 3 | 163 |
| D-2823 | 99 | 1 | 186 |
| D-2858 | 106 | 1 | 197 |

### Example 4. siRNA Targeting SOD1

The passenger-guide strand duplexes of the SOD1 siRNA found to be efficacious are engineered into expression vectors and transfected into cells of the central nervous system or neuronal cell lines. Even though overhang utilized in the siRNA knockdown study is a canonical dTdT for siRNA, the overhang in the constructs may comprise any dinucleotide overhang.

The cells used may be primary cells or derived from induced pluripotent stem cells (iPS cells).

SOD1 knockdown is then measured and deep sequencing performed to determine the exact passenger and guide strand processed from each construct administered in the expression vector.

A guide to passenger strand ratio is calculated to determine the efficiency of knockdown, e.g., of RNA Induced Silencing Complex (RISC) processing.

The N-terminus is sequenced to determine the cleavage site and to determine the percent homogeneous cleavage of the target. It is expected that cleavage will be higher than 90 percent.

HeLa cells are co-transfected in a parallel study to analyze in vitro knockdown of SOD1. A luciferase construct is used as a control to determine off-target effects.

Deep sequencing is again performed.

### Example 5. Passenger and Guide Sequences Targeting SOD1

According to the present invention, SOD1 siRNAs were designed. These are given in Tables 11A and 11B. The passenger and guide strands are described in the tables. In Tables 11A and 11B, the “miR” component of the name of the sequence does not necessarily correspond to the sequence numbering of miRNA genes (e.g., VOYmiR-101 is the name of the sequence and does not necessarily mean that miR-101 is part of the sequence).

TABLE 11A
Passenger and Guide Sequences (5′-3′)

| | Duplex | | | Passenger | AS | | Guide |
|---|---|---|---|---|---|---|---|
| Name | ID | SS ID | Passenger | SEQ ID | ID | Guide | SEQ ID |
| VOYpre-001_D- | D-2910 | 7752 | CAAUGUG | 342 | 7753 | UUUGU | 343 |
| 2806_Starting | | | ACUGCUG | | | CAGCA | |
| construct (18 native | | | ACAACCC | | | GUCAC | |
| nucleotides and | | | | | | AUUGU | |
| position 19 is C; 3′ | | | | | | U | |
| terminal CC | | | | | | | |
| dinucleotide) | | | | | | | |
| VOYpre-002_D- | D-2911 | 7754 | CAAUGUG | 344 | 7753 | UUUGU | 343 |
| 2806_p19MMU | | | ACUGCUG | | | CAGCA | |
| (position 19 U to form | | | ACAAUCC | | | GUCAC | |
| mismatch) | | | | | | AUUGU | |
| | | | | | | U | |
| VOYpre-003_D- | D-2912 | 7755 | CAAUGUG | 345 | 7753 | UUUGU | 343 |
| 2806_p19GUpair | | | ACUGCUG | | | CAGCA | |
| (position 19 is G to | | | ACAAGCC | | | GUCAC | |
| form GU pair) | | | | | | AUUGU | |
| | | | | | | U | |
| VOYpre-004_D- | D-2913 | 7756 | CAAUGUG | 346 | 7753 | UUUGU | 343 |
| 2806_p19AUpair | | | ACUGCUG | | | CAGCA | |
| (position 19 is A to | | | ACAAACC | | | GUCAC | |
| form AU pair) | | | | | | AUUGU | |
| | | | | | | U | |
| VOYpre-005_D- | D-2914 | 7757 | CAAUGUG | 347 | 7753 | UUUGU | 343 |
| 2806_CMM (central | | | ACAGCUG | | | CAGCA | |
| mismatch) | | | ACAAACC | | | GUCAC | |
| | | | | | | AUUGU | |
| | | | | | | U | |
| VOYpre-006_D- | D-2915 | 7758 | CAAUGUG | 348 | 7753 | UUUGU | 343 |
| 2806_p19DEL | | | ACUGCUG | | | CAGCA | |
| (position 19 deleted) | | | ACAACC | | | GUCAC | |
| | | | | | | AUUGU | |
| | | | | | | U | |
| VOYpre-007_D- | D-2916 | 7759 | CAAUGUG | 349 | 7753 | UUUGU | 343 |
| 2806_p19ADD | | | ACUGCUG | | | CAGCA | |
| (nucleotide added at | | | ACAAUCC | | | GUCAC | |
| position 19; addition is | | | C | | | AUUGU | |
| U; keep C and | | | | | | U | |
| terminal CC | | | | | | | |
| dinucleotide) | | | | | | | |
| VOYpre-008_D- | D-2917 | 7752 | CAAUGUG | 342 | 7753 | UUUGU | 343 |
| 2806_Uloop | | | ACUGCUG | | | CAGCA | |
| | | | ACAACCC | | | GUCAC | |
| | | | | | | AUUGU | |
| | | | | | | U | |
| VOYpre-009_D- | D-2918 | 7752 | CAAUGUG | 342 | 7753 | UUUGU | 343 |
| 2806_AUloop | | | ACUGCUG | | | CAGCA | |
| | | | ACAACCC | | | GUCAC | |
| | | | | | | AUUGU | |
| | | | | | | U | |
| VOYpre-010_D- | D-2919 | 7760 | CAAUGUG | 350 | 7753 | UUUGU | 343 |
| 2806_mir-22-loop | | | ACUGCUG | | | CAGCA | |
| | | | ACAACAC | | | GUCAC | |
| | | | | | | AUUGU | |
| | | | | | | U | |
| VOYmiR-101_pre- | D-2923 | 7752 | CAAUGUG | 342 | 7753 | UUUGU | 343 |
| 001 hsa-mir-155; D- | | | ACUGCUG | | | CAGCA | |
| 2806 | | | ACAACCC | | | GUCAC | |
| | | | | | | AUUGU | |
| | | | | | | U | |
| VOYmiR-102_pre- | D-2924 | 7752 | CAAUGUG | 342 | 7753 | UUUGU | 343 |
| 001 Engineered; D- | | | ACUGCUG | | | CAGCA | |
| 2806; let-7b stem | | | ACAACCC | | | GUCAC | |
| | | | | | | AUUGU | |
| | | | | | | U | |
| VOYmiR-103_pre- | D-2925 | 7754 | CAAUGUG | 344 | 7753 | UUUGU | 343 |
| 002 Engineered; D- | | | ACUGCUG | | | CAGCA | |
| 2806_p19MMU; let- | | | ACAAUCC | | | GUCAC | |
| 7b stem | | | | | | AUUGU | |
| | | | | | | U | |
| VOYmiR-104_pre- | D-2926 | 7755 | CAAUGUG | 345 | 7753 | UUUGU | 343 |
| 003 Engineered; D- | | | ACUGCUG | | | CAGCA | |
| 2806_p19GUpair; let- | | | ACAAGCC | | | GUCAC | |
| 7b stem | | | | | | AUUGU | |
| | | | | | | U | |
| VOYmiR-105_pre- | D-2927 | 7756 | CAAUGUG | 346 | 7753 | UUUGU | 343 |
| 004 Engineered; D- | | | ACUGCUG | | | CAGCA | |
| 2806_p19AUpair; let- | | | ACAAACC | | | GUCAC | |
| 7b stem | | | | | | AUUGU | |
| | | | | | | U | |
| VOYmiR-106_pre- | D-2928 | 7757 | CAAUGUG | 347 | 7753 | UUUGU | 343 |
| 005 Engineered; D- | | | ACAGCUG | | | CAGCA | |
| 2806_CMM; let-7b | | | ACAAACC | | | GUCAC | |
| stem | | | | | | AUUGU | |
| | | | | | | U | |
| VOYmiR-107_pre- | D-2929 | 7758 | CAAUGUG | 348 | 7753 | UUUGU | 343 |
| 006 Engineered; D- | | | ACUGCUG | | | CAGCA | |
| 2806_p19DEL; let-7b | | | ACAACC | | | GUCAC | |
| stem | | | | | | AUUGU | |
| | | | | | | U | |
| VOYmiR-108_pre- | D-2930 | 7765 | CAAUGUG | 355 | 7753 | UUUGU | 343 |
| 007 Engineered; D- | | | ACUGCUG | | | CAGCA | |
| 2806_p19ADD; let-7b | | | ACAAUCC | | | GUCAC | |
| stem | | | C | | | AUUGU | |
| | | | | | | U | |
| VOYmiR-109_pre- | D-2931 | 7752 | CAAUGUG | 342 | 7753 | UUUGU | 343 |
| 008 Engineered; D- | | | ACUGCUG | | | CAGCA | |
| 2806_Uloop; let-7b | | | ACAACCC | | | GUCAC | |
| stem | | | | | | AUUGU | |
| | | | | | | U | |
| VOYmiR-110_pre- | D-2932 | 7752 | CAAUGUG | 342 | 7753 | UUUGU | 343 |
| 009 Engineered; D- | | | ACUGCUG | | | CAGCA | |
| 2806_AUloop; let-7b | | | ACAACCC | | | GUCAC | |
| stem | | | | | | AUUGU | |
| | | | | | | U | |
| VOYmiR-111_pre- | D-2933 | 7760 | CAAUGUG | 350 | 7753 | UUUGU | 343 |
| 010 Engineered; D- | | | ACUGCUG | | | CAGCA | |
| 2806_mir-22-loop; let- | | | ACAACAC | | | GUCAC | |
| 7b stem | | | | | | AUUGU | |
| | | | | | | U | |
| VOYmiR-112_pre- | D-2934 | 7752 | CAAUGUG | 342 | 7753 | UUUGU | 343 |
| 001 Engineered; PD; | | | ACUGCUG | | | CAGCA | |
| D-2806; let-7b basal- | | | ACAACCC | | | GUCAC | |
| stem instability | | | | | | AUUGU | |
| | | | | | | U | |
| VOYmiR-113_pre- | D-2935 | 7754 | CAAUGUG | 344 | 7753 | UUUGU | 343 |
| 002 Engineered; D- | | | ACUGCUG | | | CAGCA | |
| 2806_p19MMU; let- | | | ACAAUCC | | | GUCAC | |
| 7b basal-stem | | | | | | AUUGU | |
| instability | | | | | | U | |
| VOYmiR-114_pre- | D-2936 | 7757 | CAAUGUG | 347 | 7753 | UUUGU | 343 |
| 005 Engineered; D- | | | ACAGCUG | | | CAGCA | |
| 2806_CMM; let-7b | | | ACAAACC | | | GUCAC | |
| basal-stem instability | | | | | | AUUGU | |
| | | | | | | U | |
| VOYmiR-115_pre- | D-2937 | 7760 | CAAUGUG | 350 | 7753 | UUUGU | 343 |
| 010 Engineered; D- | | | ACUGCUG | | | CAGCA | |
| 2806_mir-22-loop; let- | | | ACAACAC | | | GUCAC | |
| 7b basal-stem | | | | | | AUUGU | |
| instability | | | | | | U | |
| VOYmiR-116_pre- | D-2938 | 7755 | CAAUGUG | 345 | 7753 | UUUGU | 343 |
| 003 Engineered; D- | | | ACUGCUG | | | CAGCA | |
| 2806_p19GUpair; let- | | | ACAAGCC | | | GUCAC | |
| 7b basal-stem | | | | | | AUUGU | |
| instability | | | | | | U | |
| VOYmiR-117_pre- | D-2939 | 7766 | CGACGAA | 356 | 7767 | UCGCA | 357 |
| 001 Engineered; D- | | | GGCCGUG | | | CACGG | |
| 2757; let-7b stem | | | UGCGCCC | | | CCUUC | |
| | | | | | | GUCGU | |
| | | | | | | U | |
| VOYmiR-118_pre- | D-2940 | 7768 | UGACUUG | 358 | 7769 | UCCAC | 359 |
| 001 Engineered; D- | | | GGCAAAG | | | CUUUG | |
| 2823; let-7b stem | | | GUGGCCC | | | CCCAA | |
| | | | | | | GUCAU | |
| | | | | | | U | |
| VOYmiR-119_pre- | D-2941 | 7770 | AACUCAU | 360 | 7771 | UCAGG | 361 |
| 001 Engineered; D- | | | CUGUUAU | | | AUAAC | |
| 2866; let-7b stem | | | CCUGCCC | | | AGAUG | |
| | | | | | | AGUUU | |
| | | | | | | U | |
| VOYmiR-127 | D-2942 | 7752 | CAAUGUG | 342 | 7753 | UUUGU | 343 |
| | | | ACUGCUG | | | CAGCA | |
| | | | ACAACCC | | | GUCAC | |
| | | | | | | AUUGU | |
| | | | | | | U | |
| VOYmiR-102.860 | D-2943 | 7772 | CCCCUUA | 362 | 7773 | UAACA | 363 |
| | | | ACUCAUC | | | GAUGA | |
| | | | UGUUCCC | | | GUUAA | |
| | | | | | | GGGGU | |
| | | | | | | U | |
| VOYmiR102.861 | D-2944 | 7774 | CCCUUAA | 364 | 7775 | UUAAC | 365 |
| | | | CUCAUCU | | | AGAUG | |
| | | | GUUACCC | | | AGUUA | |
| | | | | | | AGGGU | |
| | | | | | | U | |
| VOYmiR-102.866 | D-2945 | 7776 | AACUCAU | 366 | 7771 | UCAGG | 361 |
| | | | CUGUUAU | | | AUAAC | |
| | | | CUUGCCC | | | AGAUG | |
| | | | | | | AGUUU | |
| | | | | | | U | |
| VOYmiR-102.870 | D-2946 | 7777 | GCUGUGG | 367 | 7778 | UAGGA | 368 |
| | | | AAAUGUA | | | UACAU | |
| | | | UCUUCCC | | | UUCUA | |
| | | | | | | CAGCU | |
| | | | | | | U | |
| VOYmiR-102.823 | D-2947 | 7779 | UGACUUG | 369 | 7769 | UCCAC | 359 |
| | | | GGCAAAG | | | CUUUG | |
| | | | GUGAGCC | | | CCCAA | |
| | | | | | | GUCAU | |
| | | | | | | U | |
| VOYmiR-104.860 | D-2948 | 7780 | CCCCUUA | 370 | 7773 | UAACA | 363 |
| | | | ACUCAUC | | | GAUGA | |
| | | | UGUUGCC | | | GUUAA | |
| | | | | | | GGGGU | |
| | | | | | | U | |
| VOYmiR-104.861 | D-2949 | 7781 | CCCUUAA | 371 | 7775 | UUAAC | 365 |
| | | | CUCAUCU | | | AGAUG | |
| | | | GUUAGCC | | | AGUUA | |
| | | | | | | AGGGU | |
| | | | | | | U | |
| VOYmiR-104.866 | D-2950 | 7782 | AACUCAU | 372 | 7771 | UCAGG | 361 |
| | | | CUGUUAU | | | AUAAC | |
| | | | CUUAGCC | | | AGAUG | |
| | | | | | | AGUUU | |
| | | | | | | U | |
| VOYmiR-104.870 | D-2951 | 7783 | GCUGUGG | 373 | 7778 | UAGGA | 368 |
| | | | AAAUGUA | | | UACAU | |
| | | | UCUUGCC | | | UUCUA | |
| | | | | | | CAGCU | |
| | | | | | | U | |
| VOYmiR-104.823 | D-2952 | 7784 | UGACUUG | 374 | 7769 | UCCAC | 359 |
| | | | GGCAAAG | | | CUUUG | |
| | | | GUAGGCC | | | CCCAA | |
| | | | | | | GUCAU | |
| | | | | | | U | |
| VOYmiR-109.860 | D-2953 | 7772 | CCCCUUA | 362 | 7773 | UAACA | 363 |
| | | | ACUCAUC | | | GAUGA | |
| | | | UGUUCCC | | | GUUAA | |
| | | | | | | GGGGU | |
| | | | | | | U | |
| VOYmiR-104.861 | D-2954 | 7774 | CCCUUAA | 364 | 7775 | UUAAC | 365 |
| | | | CUCAUCU | | | AGAUG | |
| | | | GUUACCC | | | AGUUA | |
| | | | | | | AGGGU | |
| | | | | | | U | |
| VOYmiR-104.866 | D-2955 | 7776 | AACUCAU | 366 | 7771 | UCAGG | 361 |
| | | | CUGUUAU | | | AUAAC | |
| | | | CUUGCCC | | | AGAUG | |
| | | | | | | AGUUU | |
| | | | | | | U | |
| VOYmiR-109.870 | D-2956 | 7777 | GCUGUGG | 367 | 7778 | UAGGA | 368 |
| | | | AAAUGUA | | | UACAU | |
| | | | UCUUCCC | | | UUCUA | |
| | | | | | | CAGCU | |
| | | | | | | OD | |
| VOYmiR-109.823 | D-2957 | 7779 | UGACUUG | 369 | 7769 | UCCAC | 359 |
| | | | GGCAAAG | | | CUUUG | |
| | | | GUGAGCC | | | CCCAA | |
| | | | | | | GUCAU | |
| | | | | | | U | |
| VOYmiR-114.860 | D-2958 | 7785 | CCCCUUA | 375 | 7773 | UAACA | 363 |
| | | | ACACAUC | | | GAUGA | |
| | | | UGUUACC | | | GUUAA | |
| | | | | | | GGGGU | |
| | | | | | | U | |
| VOYmiR-114.861 | D-2959 | 7786 | CCCUUAA | 376 | 7775 | UUAAC | 365 |
| | | | CUGAUCU | | | AGAUG | |
| | | | GUUAACC | | | AGUUA | |
| | | | | | | AGGGU | |
| | | | | | | U | |
| VOYmiR-114.866 | D-2960 | 7787 | AACUCAU | 377 | 7771 | UCAGG | 361 |
| | | | CUCUUAU | | | AUAAC | |
| | | | CUUGCCC | | | AGAUG | |
| | | | | | | AGUUU | |
| | | | | | | U | |
| VOYmiR-114.870 | D-2961 | 7788 | GCUGUGG | 378 | 7778 | UAGGA | 368 |
| | | | AAUUGUA | | | UACAU | |
| | | | UCUUGCC | | | UUCUA | |
| | | | | | | CAGCU | |
| | | | | | | U | |
| VOYmiR-114.823 | D-2962 | 7789 | UGACUUG | 379 | 7769 | UCCAC | 359 |
| | | | GGGAAAG | | | CUUUG | |
| | | | GUGAGCC | | | CCCAA | |
| | | | | | | GUCAU | |
| | | | | | | U | |
| VOYmiR-116.860 | D-2963 | 7780 | CCCCUUA | 370 | 7773 | UAACA | 363 |
| | | | ACUCAUC | | | GAUGA | |
| | | | UGUUGCC | | | GUUAA | |
| | | | | | | GGGGU | |
| | | | | | | U | |
| VOYmiR-116.861 | D-2964 | 7781 | CCCUUAA | 371 | 7775 | UUAAC | 365 |
| | | | CUCAUCU | | | AGAUG | |
| | | | GUUAGCC | | | AGUUA | |
| | | | | | | AGGGU | |
| | | | | | | U | |
| VOYmiR-116.866 | D-2965 | 7790 | AACUCAU | 380 | 7771 | UCAGG | 361 |
| | | | CUGUUAU | | | AUAAC | |
| | | | CUUGGCC | | | AGAUG | |
| | | | | | | AGUUU | |
| | | | | | | U | |
| VOYmiR-116.870 | D-2966 | 7783 | GCUGUGG | 373 | 7778 | UAGGA | 368 |
| | | | AAAUGUA | | | UACAU | |
| | | | UCUUGCC | | | UUCUA | |
| | | | | | | CAGCU | |
| | | | | | | U | |
| VOYmiR-116.823 | D-2967 | 7784 | UGACUUG | 374 | 7769 | UCCAC | 359 |
| | | | GGCAAAG | | | CUUUG | |
| | | | GUAGGCC | | | CCCAA | |
| | | | | | | GUCAU | |
| | | | | | | U | |
| VoymiR-127.860 | D-2968 | 7791 | CCCCUUA | 381 | 7773 | UAACA | 363 |
| | | | ACUCAUU | | | GAUGA | |
| | | | UGUUCCC | | | GUUAA | |
| | | | | | | GGGGU | |
| | | | | | | U | |
| VoymiR-127.861 | D-2969 | 7774 | CCCUUAA | 364 | 7775 | UUAAC | 365 |
| | | | CUCAUCU | | | AGAUG | |
| | | | GUUACCC | | | AGUUA | |
| | | | | | | AGGGU | |
| | | | | | | U | |
| VoymiR-127.866 | D-2970 | 7776 | AACUCAU | 366 | 7771 | UCAGG | 361 |
| | | | CUGUUAU | | | AUAAC | |
| | | | CUUGCCC | | | AGAUG | |
| | | | | | | AGUUU | |
| | | | | | | U | |
| VoymiR-127.870 | D-2971 | 7777 | GCUGUGG | 367 | 7778 | UAGGA | 368 |
| | | | AAAUGUA | | | UACAU | |
| | | | UCUUCCC | | | UUCUA | |
| | | | | | | CAGCU | |
| | | | | | | U | |
| VoymiR-127.823 | D-2972 | 7792 | UGACUUG | 382 | 7769 | UCCAC | 359 |
| | | | GGCAAAG | | | CUUUG | |
| | | | GUAGCCC | | | CCCAA | |
| | | | | | | GUCAU | |
| | | | | | | U | |
| VOYmiR-120 | D-2973 | 7793 | CAAUGUG | 383 | 7794 | UUUGU | 384 |
| | | | ACUGCUG | | | CAGCA | |
| | | | ACAAA | | | GUCAC | |
| | | | | | | AUUGU | |
| | | | | | | C | |

TABLE 11B
Passenger and Guide Sequences (5′-3′)

| | Duplex | | | Passenger | AS | | Guide |
|---|---|---|---|---|---|---|---|
| Name | ID | SS ID | Passenger | SEQ ID | ID | Guide | SEQ ID |
| VOYpre-011_D- | D-2920 | 7761 | UUUGUCA | 351 | 7762 | CAAUG | 352 |
| 2806_passenger- | | | GCAGUCA | | | UGACU | |
| guide strand swap | | | CAUUGUC | | | GCUGA | |
| with terminal 3′ C | | | | | | CAAAU | |
| on passenger | | | | | | C | |
| strand | | | | | | | |
| VOYpre-012_D- | D-2921 | 7761 | UUUGUCA | 351 | 7763 | CAAUG | 353 |
| 2806_passenger- | | | GCAGUCA | | | UGACU | |
| guide strand swap | | | CAUUGUC | | | GCUGA | |
| with terminal 3′ C | | | | | | CAAUU | |
| on passenger | | | | | | C | |
| strand | | | | | | | |
| VOYpre-013_D- | D-2922 | 7764 | UUUGUCA | 354 | 7762 | CAAUG | 352 |
| 2806_passenger- | | | GCAGUCA | | | UGACU | |
| guide strand swap | | | CAUUGAC | | | GCUGA | |
| with terminal 3′ C | | | | | | CAAAU | |
| on passenger | | | | | | C | |
| strand | | | | | | | |

### Example 6. SOD1 siRNA Constructs in AAV-miRNA Vectors

The passenger-guide strand duplexes of the SOD1 siRNA listed in Table 11 are engineered into AAV-miRNA expression vectors. The construct from ITR to ITR, recited 5′ to 3′, comprises a mutant ITR, a promoter (either a CMV, a U6 or the CB6 promoter (which includes a CMVie enhancer, a CBA promoter and an SV40 intron), the passenger and guide strand (with a loop between the passenger and guide strand, a 5′ flanking region before the passenger strand and a 3′ flanking region after the guide strand) from Table 11, a rabbit globin polyA and wild type ITR. In vitro and in vivo studies are performed to test the efficacy of the AAV-miRNA expression vectors.

### Example 7. Activity of Constructs in Hela Cells

Seven of the SOD1 siRNA constructs described in Example 6 (VOYmiR-103, VOYmiR-105, VOYmiR-108, VOYmiR-114, VOYmiR-119, VOYmiR-120, and VOYmiR-127) and a control of double stranded mCherry were transfected in HeLa to test the activity of the constructs.

### A. Passenger and Guide Strand Activity

The seven SOD1 siRNA constructs and a control of double stranded mCherry were transfected into HeLa cells. After 48 hours the endogenous mRNA expression was evaluated. All seven of the SOD1 siRNA constructs showed high activity of the guide strand with 75-80% knock-down and low to no activity of the passenger strand. Guide strands of the SOD1 siRNA candidate vectors showed high activity, yielding 75-80% knockdown of SOD1, while passenger strands demonstrated little to no activity.

### B. Activity of Constructs on SOD1

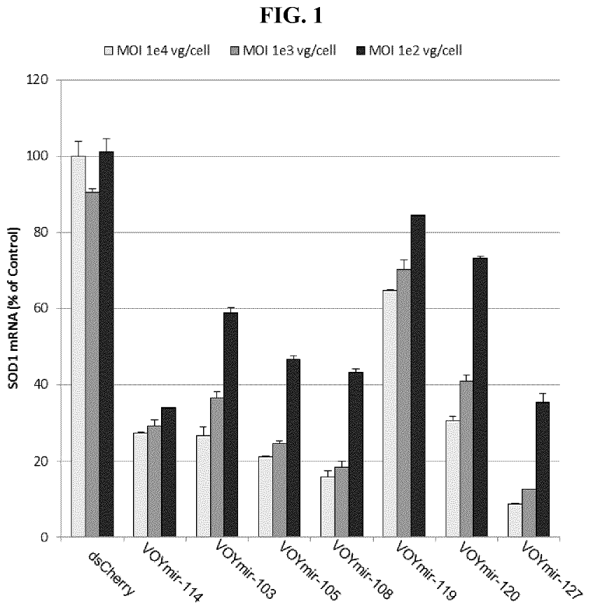

The seven SOD1 siRNA constructs and a control of double stranded mCherry (dsCherry) were transfected into HeLa cells at a MOI of 1e4 vg/cell, 1e3 vg/cell, or 1e2 vg/cell. After 72 hours the endogenous mRNA expression was evaluated. All seven of the SOD1 siRNA constructs showed efficient knock-down at 1e3 vg/cell. Most of the SOD1 siRNA constructs showed high activity (75-80% knock-down) as shown in FIG. 1.

### Example 8. Activity of Constructs in HEK Cells

Thirty of the SOD1 siRNA constructs described in Example 6 (VOYmiR-102.860, VOYmiR-102.861, VOYmiR-102.866, VOYmiR-102.870, VOYmiR-102.823, VOYmiR-104.860, VOYmiR-104.861, VOYmiR-104.866, VOYmiR-104.870, VOYmiR-104.823, VOYmiR-109.860, VOYmiR-109.861, VOYmiR-109.866, VOYmiR-109.870, VOYmiR-109.823, VOYmiR-114.860, VOYmiR-114.861, VOYmiR-114.866, VOYmiR-114.870, VOYmiR-114.823, VOYmiR-116.860, VOYmiR-116.861, VOYmiR-116.866, VOYmiR-116.870, VOYmiR-116.823, VOYmiR-127.860, VOYmiR-127.861, VOYmiR-127.866, VOYmiR-127.870, VOYmiR-127.823) and a control of VOYmiR-114 and double stranded mCherry were transfected in cells to test the activity of the constructs.

### A. Passenger and Guide Strand Activity in HEK293

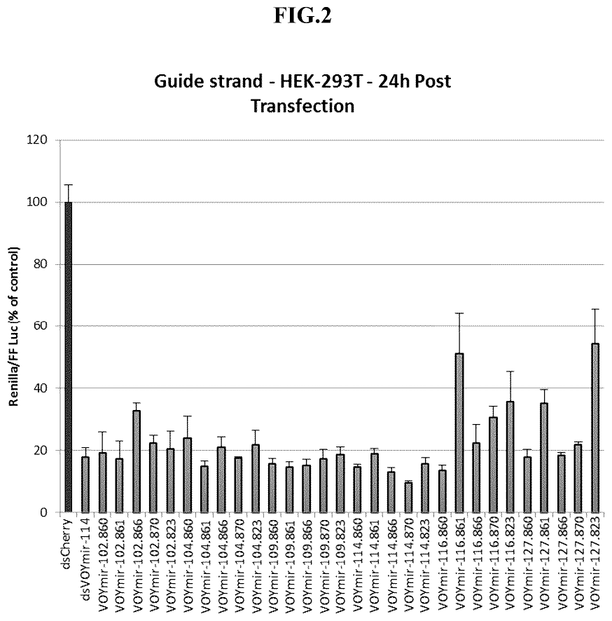

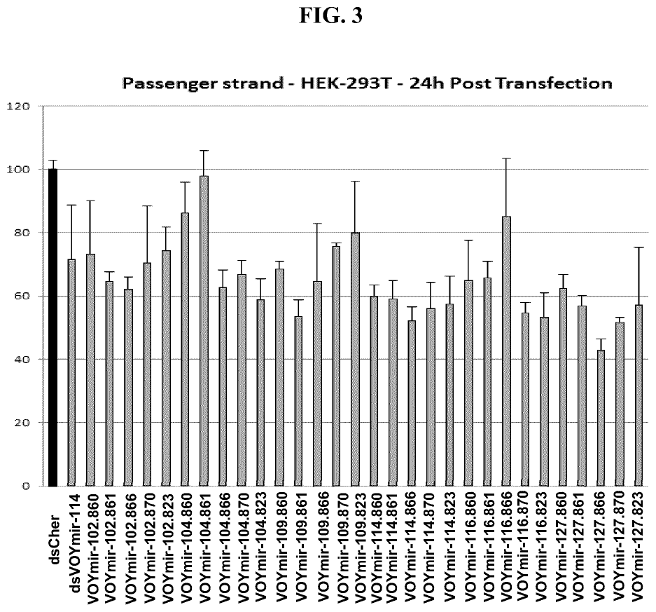

The thirty constructs and two controls were transfected into HEK293T cells. After 24 hours the endogenous mRNA expression was evaluated. Most of the constructs showed high activity of the guide strand (FIG. 2) and low to no activity of the passenger strand (FIG. 3).

### B. Passenger and Guide Strand Activity in HeLa

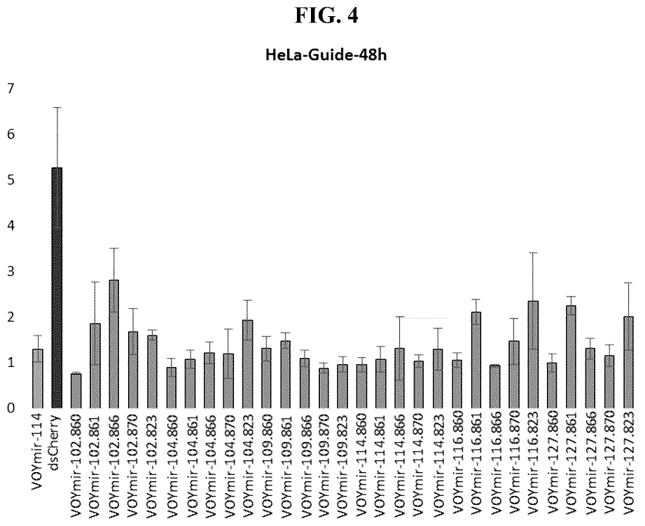

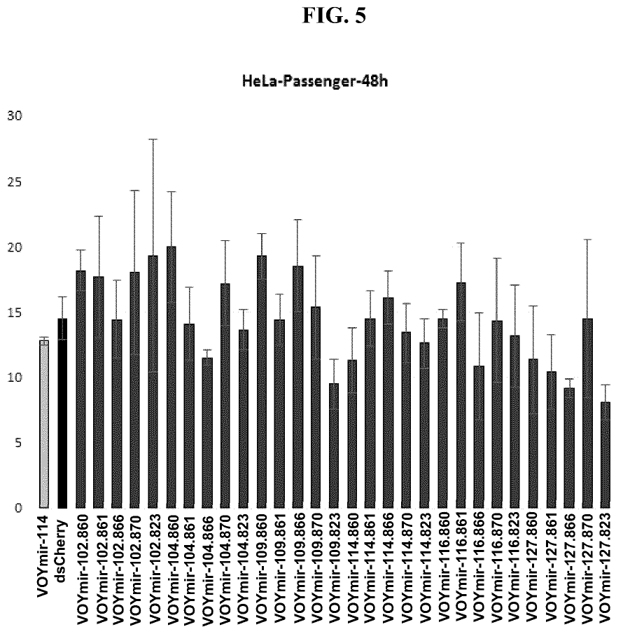

The thirty constructs and two controls were transfected into Hela cells. After 48 hours the endogenous mRNA expression was evaluated. Most of the constructs showed high activity of the guide strand (FIG. 4) and low to no activity of the passenger strand (FIG. 5).

### C. HeLa and HEK293 Correlation

The knock-down of the thirty constructs were similar between the HeLa and HEK293 cells. The thirty constructs showed knock-down for the guide strand for the constructs (See FIGS. 2 and 4). Most of the guide strands of the constructs showed 70-90% knock-down.

### D. Capsid Selection

The top constructs from the HeLa and HEK293 are packaged in AAVs and will undergo HeLa infection. To determine the best AAV to package the constructs, mCherry packaged in either AAV2 or AAV-DJ8 was infected into Hela cells at a MOI of 10 vg/cell, 1e2 vg/cell, 1e3 vg/cell, 1e4 vg/cell or 1e5 vg/cell and the expression was evaluated at 40 hours. AAV2 was selected as the capsid to package the top constructs.

### E. AAV2 Production

The top constructs from the HeLa and HEK293 are packaged in AAV2 (1.6 kb) and a control of double stranded mCherry (dsmCherry) was also packaged. The packaged constructs underwent Idoixanol purification prior to analysis. The AAV titer is shown in Table 12.

TABLE 12
AAV Titer

| Construct | AAV Titer (genomes per ul) |
|---|---|
| VOYmir-102.860 | 5.5E+08 |
| VOYmir-102.861 | 1.0E+09 |
| VOYmir-102.823 | 9.1E+08 |
| VOYmir-104.861 | 1.2E+09 |
| VOYmir-104.866 | 8.0E+08 |
| VOYmir-104.823 | 5.7E+08 |
| VOYmir-109.860 | 3.1E+08 |
| VOYmir-109.861 | 8.9E+08 |
| VOYmir-109.866 | 6.0E+08 |
| VOYmir-109.823 | 6.0E+08 |
| VOYmir-114.860 | 4.7E+08 |
| VOYmir-114.861 | 3.7E+08 |
| VOYmir-114.866 | 1.0E+09 |
| VOYmir-144.823 | 1.7E+09 |
| VOYmir-116.860 | 1.0E+09 |
| VOYmir-116.866 | 9.1E+08 |
| VOYmir-127.860 | 1.2E+09 |
| VOYmir-127.866 | 9.0E+08 |
| dsmCherry | 1.2E+09 |

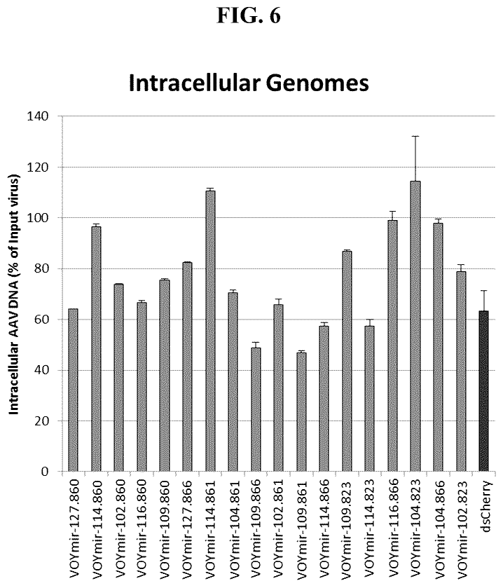

The effect of transduction on SOD1 knock-down in Hela cells is shown in FIG. 6. In addition, in Hela cells, a larger MOI (1.0E+04 compared to 1.0E+05) did not show increased knock-down for every construct.

### F. Activity of Constructs in Human Motor Neuron Progenitors (HMNPs)

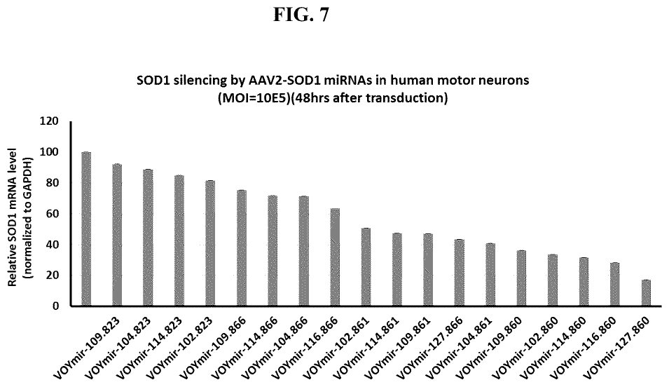

The top 18 pri-miRNA constructs as described in Example 8E and a control of mCherry were infected into human motor neuron progenitor (HMNP) cells at a MOI of 10E5. After 48 hours the endogenous mRNA expression was evaluated. About half of the constructs gave greater than 50% silencing of SOD1 in HMNPs and 4 of those gave greater than 70% silencing (FIG. 7).

### G. Construct Selection for In Vivo Studies

The top twelve constructs are selected which had a major effect on the target sequence and a minor effect on the cassette. These constructs packaged in AAV-rh10 capsids are formulated for injection and administered in mammals to study the in vivo effects of the constructs.

### Example 9. In Vitro Study of Constructs

The 18 constructs and mCherry control described in Example 8D packaged in AAV2 were used for this study. For this study, HEK293T cells (Fisher Scientific, Cat. #HCL4517) in culture medium (500 ml of DMEM/F-12 GLUTAMAX™ supplement (Life Technologies, Cat #. 10565-018), 50 ml FBS (Life Technologies, Cat #. 16000-044, lot: 1347556), 5 ml MEM Non-essential amino acids solution (100×) (Cat. #11140-050) and 5 ml HEPES (1M) (Life Technologies, Cat #. 15630-080)), U251MG cells (P18) (Sigma, Cat #. 09063001-1VL) in culture medium (500 ml of DMEM/F-12 GLUTAMAX™ supplement (Life Technologies, Cat #. 10565-018), 50 ml FBS (Life Technologies, Cat #. 16000-044, lot: 1347556), 5 ml MEM Non-essential amino acids solution (100×) (Cat. #11140-050) and 5 ml HEPES (1M) (Life Technologies, Cat #. 15630-080)) or normal human astrocyte (HA) (Lonza, Cat #CC-2565) in culture medium (ABM Basal Medium 500 ml (Lonza, Cat #. CC-3186) supplemented with AGM SingleQuot Kit Suppl. & Growth Factors (Lonza, Cat #. CC-4123)) were used to test the constructs. HEK293T cells (5×10E4 cells/well in 96 well plate), U251MG cells (2×10E4 cells/well in 96 well plate) and HA cells (2×10E4 cells/well in 96 well plate) were seeded and the MOI used for infection of cells was 1.0E+05. After 48 hours the cells were analyzed and the results are shown in Table 13.

TABLE 13
Relative SOD1 mRNA level

| | Relative SOD1 mRNA Level | | |
|---|---|---|---|
| | (%) (Normalized to GAPDH) | | |
| Construct | HEK293T | U251MG | HA |
| VOYmiR-102.823 | 19.5 | 49.6 | 87.3 |
| VOYmiR-102.860 | 1.7 | 5.3 | 19.2 |
| VOYmiR-102.861 | 1.1 | 13.9 | 42.6 |
| VOYmiR-104.823 | 49.9 | 69.6 | 102.7 |
| VOYmiR-104.861 | 1.0 | 10.7 | 36.3 |
| VOYmiR-104.866 | 12.3 | 54.6 | 85.5 |
| VOYmiR-109.823 | 23.0 | 46.1 | 84.6 |
| VOYmiR-109.860 | 1.9 | 8.3 | 35.6 |
| VOYmiR-109.861 | 1.9 | 22.7 | 57.3 |
| VOYmiR-109.866 | 4.1 | 38.5 | 67.9 |
| VOYmiR-114.823 | 19.3 | 44.7 | 82.3 |
| VOYmiR-114.860 | 1.4 | 4.7 | 17.6 |
| VOYmiR-114.861 | 1.1 | 9.7 | 48.1 |
| VOYmiR-114.866 | 4.0 | 38.7 | 78.2 |
| VOYmiR-116.860 | 1.1 | 4.8 | 15.8 |
| VOYmiR-116.866 | 5.5 | 40.2 | 73.7 |
| VOYmiR-127.860 | 1.0 | 2.1 | 7.4 |
| VOYmiR-127.866 | 1.0 | 15.4 | 43.8 |
| mCherry | 100.0 | 100.2 | 100.1 |

Greater than 80% knock-down was seen in the HEK293T cells for most constructs. More than half of the constructs showed greater than 80% knock-down in the U251MG cells and in the HA cells.

### Example 10. Dose Dependent SOD1 Lowering

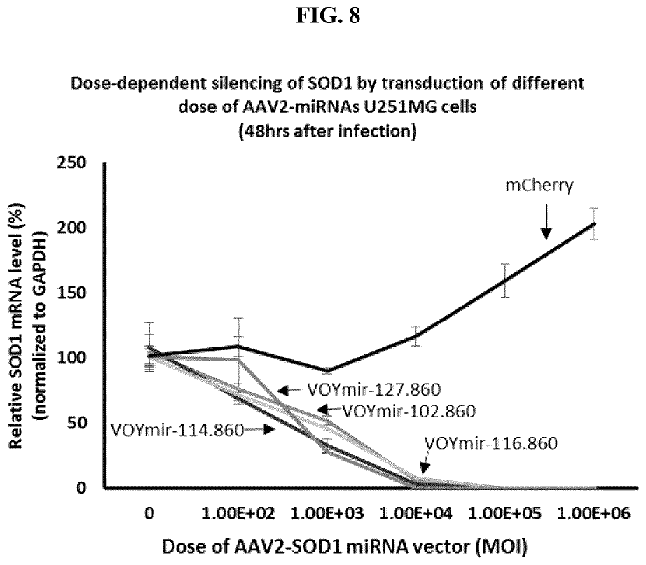

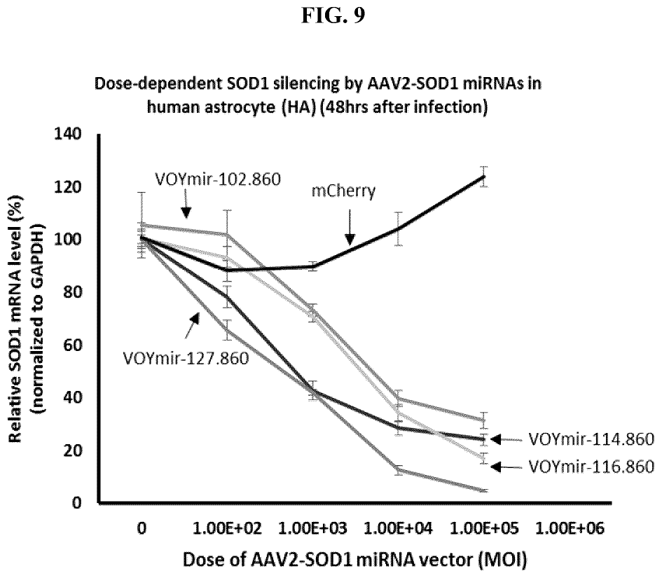

Four of the top 18 pri-miRNA constructs as described in Example 8E and a control of mCherry were transfected into a human astrocyte cell line (U251MG) or a primary human astrocyte (HA) at an MOI of 1.0E+02, 1.0E+03, 1.0E+04, 1.0E+05 or 1.0E+06. After 48 hours the endogenous mRNA expression was evaluated and the dose-dependent silencing are shown in FIG. 8 (U251MG) and FIG. 9 (HA). For all constructs, the increase in dose also correlated to an increase in the amount of SOD1 mRNA that was knocked-down.

### Example 11. Time Course of SOD1 Knock-Down

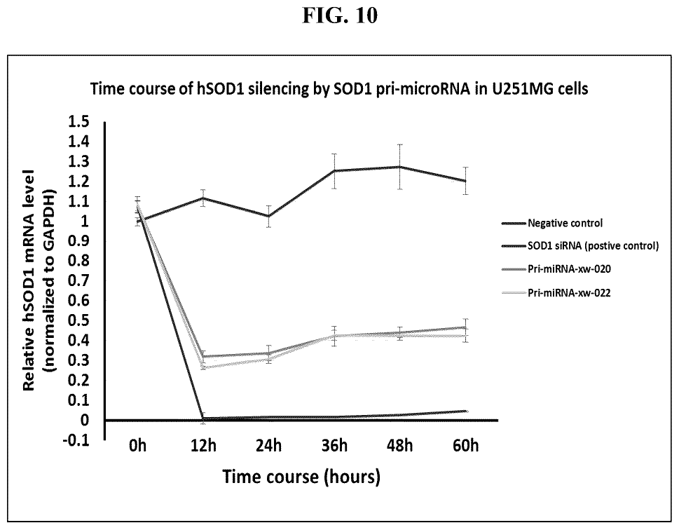

Two pri-miRNA constructs (VOYmiR-120 and VOYmiR-122), a negative control and a positive control of SOD1 siRNA were transfected into a human astrocyte cell line (U251MG). The relative SOD1 mRNA was determined for 60 hours as shown in FIG. 10. 70-75% knock-down of hSOD1 was seen for both pri-miR constructs after Nucleofector transfection, with the greatest knock-down seen in the 12-24 hour window.

### Example 12. SOD1 Knock-Down and Stand Percentages

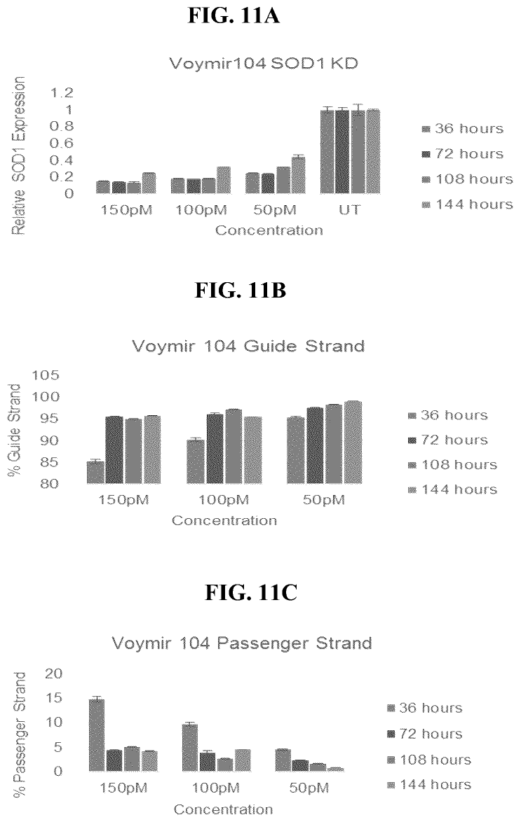

VOYmiR-104 was transfected into HeLa cells at a concentration of 50 pM, 100 pM and 150 pM and compared to untreated (UT) cells. The relative SOD1 mRNA, the percent of the guide strand and the percent of the passenger strand was determined at 36, 72, 108 and 144 hours as shown in FIGS. 11A-11C. The highest concentration (150 pM) showed the greatest reduction in expression, but all three doses showed a significant reduction in the expression of SOD1.

### Example 13. Constructs Targeting SOD1

Constructs were designed for Dog SOD1 and the constructs are given in Table 14. Dog SOD1 is 100% conserved with human in the region targeted in the present invention. The passenger and guide sequences are described in the table. In Table 14, the “miR” component of the name of the sequence does not necessarily correspond to the sequence numbering of miRNA genes (e.g., dVOYmiR-102 is the name of the sequence and does not necessarily mean that miR-102 is part of the sequence).

TABLE 14
Dog sequences (5′-3′)

| | Duplex | SS | | Passenger | AS | | Guide |
|---|---|---|---|---|---|---|---|
| Name | ID | ID | Passenger | SEQ ID | ID | Guide | SEQ ID |
| dVOYmiR- | D-2974 | 7795 | GCAGGUCC | 385 | 7796 | GAUUAAAG | 386 |
| 102.788 | | | UCACUUUA | | | UGAGGACC | |
| | | | AUGCC | | | UGCUU | |
| dVOYmiR- | D-2975 | 7797 | GGCAAUGU | 387 | 7798 | UGUCAGCA | 388 |
| 102.805 | | | GACUGCUG | | | GUCACAUU | |
| | | | ACCCC | | | GCCUU | |
| dVOYmiR- | D-2976 | 7799 | GCAGGUCC | 389 | 7796 | GAUUAAAG | 386 |
| 104.788 | | | UCACUUUA | | | UGAGGACC | |
| | | | AUUCC | | | UGCUU | |
| dVOYmiR- | D-2977 | 7800 | GGCAAUGU | 390 | 7798 | UGUCAGCA | 388 |
| 104.805 | | | GACUGCUG | | | GUCACAUU | |
| | | | AUGCC | | | GCCUU | |
| dVOYmiR- | D-2978 | 7801 | GCAGGUCC | 391 | 7796 | GAUUAAAG | 386 |
| 109.788 | | | UCACUUUA | | | UGAGGACC | |
| | | | AUCCC | | | UGCUU | |
| dVOYmiR- | D-2979 | 7802 | GGCAAUGU | 392 | 7798 | UGUCAGCA | 388 |
| 109.805 | | | GACUGCUG | | | GUCACAUU | |
| | | | AUACC | | | GCCUU | |
| dVOYmiR- | D-2980 | 7803 | GCAGGUCC | 393 | 7796 | GAUUAAAG | 386 |
| 114.788 | | | UGACUUUA | | | UGAGGACC | |
| | | | AUCCC | | | UGCUU | |
| dVOYmiR- | D-2981 | 7804 | GGCAAUGU | 394 | 7798 | UGUCAGCA | 388 |
| 114.805 | | | GUCUGCUG | | | GUCACAUU | |
| | | | AUACC | | | GCCUU | |
| dVOYmiR- | D-2982 | 7801 | GCAGGUCC | 391 | 7796 | GAUUAAAG | 386 |
| 116.788 | | | UCACUUUA | | | UGAGGACC | |
| | | | AUCCC | | | UGCUU | |
| dVOYmiR- | D-2983 | 7802 | GGCAAUGU | 392 | 7798 | UGUCAGCA | 388 |
| 116.805 | | | GACUGCUG | | | GUCACAUU | |
| | | | AUACC | | | GCCUU | |
| dVoymiR- | D-2984 | 7801 | GCAGGUCC | 391 | 7805 | GAUUAAAG | 395 |
| 127.788 | | | UCACUUUA | | | UGAGGACC | |
| | | | AUCCC | | | UGCUUU | |
| dVoymiR- | D-2985 | 7802 | GGCAAUGU | 392 | 7806 | UGUCAGCA | 396 |
| 127.805 | | | GACUGCUG | | | GUCACAUU | |
| | | | AUACC | | | GCCUUU | |

### Example 14. Effect of the Position of Modulatory Polynucleotides

### A. Effect on Viral Titers

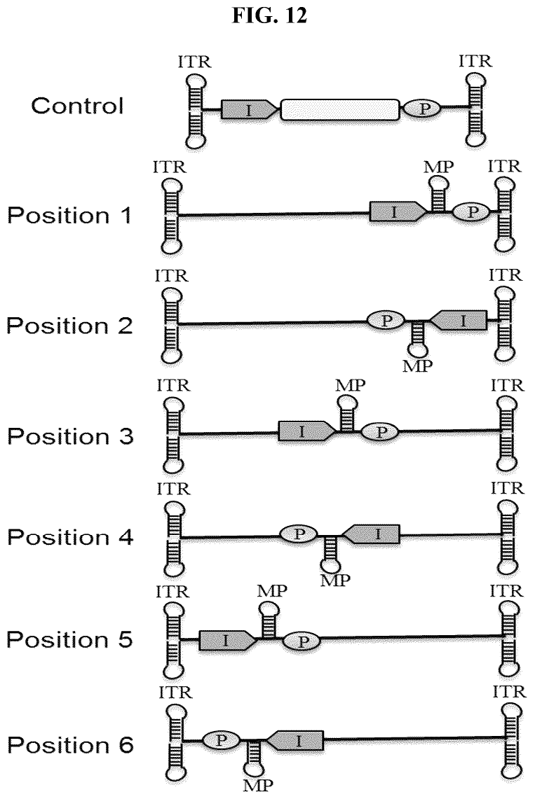

A siRNA molecule (VOYmiR-114 or VOYmiR-126) was inserted into an expression vector (genome size 2400 nucleotides; scAAV) at six different locations as shown in FIG. 12. In FIG. 12, “ITR” is the inverted terminal repeat, “I” represents intron, “P” is the polyA and “MP” is the modulatory polynucleotide comprising the siRNA molecule. The viral titers were evaluated using TaqMan PCR for the 6 position and for a control (construct without a modulatory polynucleotide; scAAV) and the results are shown in Table 15.

TABLE 15
Viral Titers

| | | Virus Titer |
|---|---|---|
| siRNA Molecule | siRNA Molecule Position | (VG per 15-cm dish) |
| VOYmiR-114 | Position 1 | 5.5E+10 |
| VOYmiR-114 | Position 2 | 5.5E+10 |
| VOYmiR-114 | Position 3 | 4.5E+10 |
| VOYmiR-114 | Position 4 | 3.7E+10 |
| VOYmiR-114 | Position 5 | 6.5E+10 |
| VOYmiR-114 | Position 6 | 2.5E+10 |
| VOYmiR-126 | Position 1 | 1.6E+10 |
| VOYmiR-126 | Position 2 | 3.2E+10 |
| VOYmiR-126 | Position 3 | 6.0E+10 |
| VOYmiR-126 | Position 4 | 1.6E+10 |
| VOYmiR-126 | Position 5 | 9.5E+09 |
| VOYmiR-126 | Position 6 | 6.0E+10 |
| — | Control | 2.1E+11 |

### B. Effect on Genome Integrity

A siRNA molecule (VOYmiR-114) was inserted into an expression vector (genome size 2400 nucleotides; scAAV) at six different locations and a control without a modulatory polynucleotide (scAAV) as shown in FIG. 12. In FIG. 12, “ITR” is the inverted terminal repeat, “I” represents intron, “P” is the polyA and “MP” is the modulatory polynucleotide comprising the siRNA molecule. Viral genomes were extracted from purified AAV preparations and run on a neutral agarose gel. Truncated genomes were seen in all constructs and the approximate percent of the truncated genomes (percent of the total) is shown in Table 16.

TABLE 16
Truncated Genomes

| Construct | % of total |
|---|---|
| Position 1 | 50 |
| Position 2 | 41 |
| Position 3 | 49 |
| Position 4 | 34 |
| Position 5 | 33 |
| Position 6 | 59 |
| Control | 9 |

Position 6 had the greatest number of truncated genomes with Position 4 and 5 having the least amount of truncated genomes.

### C. Effect on Knock-Down Efficiency

A siRNA molecule (VOYmiR-114) was inserted into an expression vector (AAV2) (genome size 2400 nucleotides; scAAV) at six different locations as shown in FIG. 12. In FIG. 12, “ITR” is the inverted terminal repeat, “I” represents intron, “P” is the polyA and “MP” is the modulatory polynucleotide comprising the siRNA molecule. Transduction of HeLa cells was conducted at 1×10⁴ vg/cell, 1×10³ vg/cell and 1×10² vg/cell. The SOD1 mRNA expression (as % of control (eGFP)) was determined 72 hours post-infection and the results are shown in Table 17.

TABLE 17
SOD1 Expression

| | SOD1 mRNA expression (% of control) | | |
|---|---|---|---|
| Construct | 1 × 10⁴ vg/cell | 1 × 10³ vg/cell | 1 × 10² vg/cell |
| Position 1 | 40 | 59 | 69 |
| Position 2 | 31 | 46 | 75 |
| Position 3 | 50 | 66 | 81 |
| Position 4 | 21 | 34 | 55 |
| Position 5 | 49 | 52 | 67 |
| Position 6 | 31 | 37 | 62 |
| Control (eGFP) | 100 | 100 | 94 |

Position 3 had the highest SOD1 mRNA expression (as % of control) and Position 4 had the lowest SOD1 mRNA expression (as % of control).

### Example 15. Effect of Genome Size

### A. Effect on Viral Titers

A siRNA molecule (VOYmiR-114) was inserted into an expression vector (genome size 2 kb; scAAV) at positions 1, 2, 5 and 6 as shown in FIG. 12. In FIG. 12, “ITR” is the inverted terminal repeat, “I” represents intron, “P” is the polyA and “MP” is the modulatory polynucleotide comprising the siRNA molecule. A double stranded control without a siRNA molecule (genome size 1.6 kb; scAAV ctrl) and a double stranded expression vector (scAAV miR114; ITR (105 nucleotide)-Promoter (˜900 nucleotides)-modulatory polynucleotide comprising the siRNA molecule (158 nucleotides)-polyA sequence (127 nucleotides) and ITR) was compared as well as a control (eGFP; scAAV) with no siRNA molecule. The viral titers were evaluated using TaqMan PCR and the results are shown in Table 18.

TABLE 18
Viral Titers

| | | |
|---|---|---|
| | | Virus Titer |
| Construct | Size | (VG per 15-cm dish) |

| | | | |
|---|---|---|---|
| Position 1 | 2 | kb | 9.5E+10 |
| Position 2 | 2 | kb | 1.2E+11 |
| scAAV miR114 | 1.6 | kb | 1.1E+11 |
| Position 5 | 2 | kb | 2.4E+10 |
| Position 6 | 2 | kb | 1.1E+11 |
| Control | 2 | kb | 2.2E+11 |

The lowest viral titers were seen with the position 5 construct and the greatest was with the position 2 construct.

### B. Effect on Genome Integrity

A siRNA molecule (VOYmiR-114) was inserted into an expression vector (genome size 2 kb; scAAV) at positions 1, 2, 5 and 6 as shown in FIG. 12. In FIG. 12, “ITR” is the inverted terminal repeat, “I” represents intron, “P” is the polyA and “MP” is the modulatory polynucleotide comprising the siRNA molecule. A double stranded control without a siRNA molecule (genome size 1.6 kb; scAAV ctrl) and a double stranded expression vector (scAAV miR114; ITR (105 nucleotide)-Promoter (˜900 nucleotides)-modulatory polynucleotide comprising the siRNA molecule (158 nucleotides)-polyA sequence (127 nucleotides) and ITR) was compared as well as a control (eGFP; scAAV) with no siRNA molecule. Truncated genomes were seen in all constructs and the approximate percent of the truncated genomes (percent of the total) is shown in Table 19.

TABLE 19
Truncated Genomes

| | | |
|---|---|---|
| Construct | Size | % of total |

| | | | |
|---|---|---|---|
| Position 1 | 2 | kb | 34 |
| Position 2 | 2 | kb | 30 |
| scAAV miR114 | 1.6 | kb | 20 |
| Position 5 | 2 | kb | 21 |
| Position 6 | 2 | kb | 46 |
| Control | 2 | kb | 5 |

All constructs were determined to have some truncated genomes.

An additional study was conducted to determine the effect of different siRNA molecules. VOYmiR-114 and VOYmiR-126 were inserted into separate expression vectors (genome size 1.6 kb; scAAV) at position 3 as shown in FIG. 12. In FIG. 12, “ITR” is the inverted terminal repeat, “I” represents intron, “P” is the polyA and “MP” is the modulatory polynucleotide comprising the siRNA molecule. For the VOYmiR-114 construct the distance between the 5′ end of the vector genome (1526 nucleotides) and the center of the modulatory polynucleotide (middle of the flexible loop) is 1115 nucleotides. For the VOYmiR-126 construct the distance between the 5′ end of the vector genome (1626 nucleotides) and the center of the modulatory polynucleotide (middle of the flexible loop) is 1164 nucleotides.

For the VOYmiR-114 construct, the viral titer (VG per 15-cm dish) was about 1.1E+11. For the VOYmiR-126 construct, the intron probe viral titer (VG per 15-cm dish) was about 1.2E+12. The control was about 2.1E+11 (VG per 15-cm dish). VOYmir-114 had about 20% truncated genomes, VOYmiR-126 has about 15% truncated genomes and the control had about 5% truncated genomes.

### Example 16. Effect of Single Stranded Constructs

### A. Effect on Viral Titers

A siRNA polynucleotide (VOYmiR-114) was inserted into an expression vector (genome size 4.7 kb; ssAAV) at positions 1, 3 and 5 as shown in FIG. 12 and there was a control also tested without a siRNA polynucleotide (genome size 2 kb; ssAAV). In FIG. 12, “ITR” is the inverted terminal repeat, “I” represents intron, “P” is the polyA and “MP” is the modulatory polynucleotide comprising the siRNA molecule. The viral titers were evaluated using TaqMan PCR and the results are shown in Table 20.

TABLE 20
Viral Titers

| | Virus Titer |
|---|---|
| Construct | (VG per 15-cm dish) |
| Position 1 | 5.0E+11 |
| Position 3 | 7.5E+11 |
| Position 5 | 3.5E+11 |
| Control | 2.5E+11 |

Position 3 showed the greatest viral titers followed by position 1 and then position 5.

### B. Effect on Genome Integrity

A siRNA molecule (VOYmiR-114) was inserted into an expression vector (genome size 4.7 kb; ssAAV) at positions 1, 3 and 5 as shown in FIG. 12 and there was a control also tested without a modulatory polynucleotide (genome size 2 kb; ssAAV). In FIG. 12, “ITR” is the inverted terminal repeat, “I” represents intron, “P” is the polyA and “MP” is the modulatory polynucleotide comprising the siRNA molecule. Viral genomes were extracted from purified AAV preparations and run on a neutral agarose gel. Truncated genomes were seen in all constructs and the approximate percent of the truncated genomes (percent of the total) is shown in Table 21.

TABLE 21
Truncated Genomes

| Construct | % of total |
|---|---|
| Position 1 | 48 |
| Position 3 | 30 |
| Position 5 | 72 |
| Control | 0 |

Position 5 had the greatest number of truncated genomes with Position 3 having the least amount of truncated genomes.

### C. Effect on Knock-Down Efficiency

A siRNA molecule (VOYmiR-114) was inserted into an expression vector (genome size 4.7 kb; ssAAV) at positions 1, 3 and 5 as shown in FIG. 12 and there was a single stranded control without a siRNA molecule (genome size 2 kb; ssAAV ctrl), a double stranded control without a siRNA molecule (genome size 1.6 kb; scAAV ctrl) and a double stranded expression vector (genome size 2.4 kb; scAAV miR114) with a siRNA molecule. In FIG. 12, “ITR” is the inverted terminal repeat, “I” represents intron, “P” is the polyA and “MP” is the modulatory polynucleotide comprising the siRNA molecule. Transduction of HeLa cells was conducted at 1×10⁴ vg/cell, 1×10³ vg/cell and 1×10² vg/cell. The SOD1 mRNA expression (as % of control (eGFP)) was determined 72 hours post-infection and the results are shown in Table 22.

TABLE 22
SOD1 Expression

| | SOD1 mRNA expression (% of control) | | |
|---|---|---|---|
| Construct | 1 × 10⁴ vg/cell | 1 × 10³ vg/cell | 1 × 10² vg/cell |
| Position 1 | 62 | 85 | 87 |
| Position 3 | 77 | 93 | 99 |
| Position 5 | 59 | 82 | 84 |
| ssAAV ctrl | 100 | 101 | 108 |
| scAAV ctrl | 95 | 97 | 102 |
| scAAV miR114 | 23 | 33 | 62 |

Position 3 had the highest SOD1 mRNA expression (as % of control), then position 1 and the single stranded constructs with the lowest SOD1 mRNA expression (as % of control) was Position 5. None of the single stranded constructs had knock-down efficiency that was as low as the double stranded control with a siRNA polynucleotide.

### Example 17. SOD1 Knock-Down In Vivo

To evaluate the in vivo biological activity of pri-miRNAs, self-complementary pri-miRNAs (VOYmiR-114.806, VOYmiR127.806, VOYmiR102.860, VOYmiR109.860, VOYmiR114.860, VOYmiR116.860, VOYmiR127.860, VOYmiR102.861, VOYmiR104.861, VOYmiR109.861, VOYmiR114.861, VOYmiR109.866, VOYmiR116.866, or VOYmiR127.866) are packaged in AAV-DJ with a CBA promoter.

In mice, these packaged pri-miRNAs or a control of vehicle only (phosphate-buffered saline with 5% sorbitol and 0.001% F-68) were administered by a 10-minute intrastriatal infusion. Female or male Tg (SOD1) 3Cje/J mice (Jackson Laboratory, Bar Harbor, ME), which express human SOD1, and of approximately 20-30 g body weight, receive unilateral injections of 5 uL test article which is targeted to the striatum (anteroposterior+0.5 mm, mediolateral+2 mm, relative to bregma; dorsoventral 3.8 mm, relative to skull surface). Test articles are injected (5 animals per test article) at 0.5 uL/min. using pre-filled, pump-regulated Hamilton micro-syringes (1701 model, 10 μl) with 33-gauge needles. At 1, 2, 3, 4 or 6 weeks following the injection, animals are sacrificed, brains are removed, and ipsilateral striata encompassing the infusion site from a 1 mm coronal slab, as well as striatal tissue from the adjacent 1 mm coronal slabs are dissected and flash frozen. Mouse tissue samples are lysed, and human SOD1 protein levels, and SOD1 and mouse GAPDH (mGAPDH) mRNA levels are quantified. SOD1 protein levels are quantified by ELISA (eBioscience (Affymetrix, San Diego, CA)), and total protein levels are quantified by BCA analysis (ThermoFisher Scientific, Waltham, MA). For each tissue sample, the level of SOD1 protein normalized to total protein is calculated as an average of 2 determinations. These normalized SOD1 protein levels are further normalized to the vehicle group, then averaged to obtain a group (treatment) average. SOD1 and mGAPDH mRNA levels are quantified by qRT-PCR. For each tissue sample, the ratio of SOD1/mGAPDH (normalized SOD1 mRNA level) is calculated as an average of 3 determinations. These ratios are then averaged to obtain a group (treatment) average. These group averages are further normalized to the vehicle group.

In non-human primates, test articles (1×10¹³-3×10¹³vg of pri-miRNA packaged in AAV-DJ with a CBA promoter) or vehicle are administered by intrathecal lumbar bolus. Female cynomolgus monkeys (Macaca fascicularis, CR Research Model Houston, Houston, TX) of approximately 2.5-8.5 kg body weight, receive implanted single intrathecal catheters with the tip of the catheter located at the lumbar spine. Test articles are administered (4 animals per test article) comprising three 1 mL bolus injections (1 mL/minute), at approximately 60-minute intervals. At 4 to 6 weeks following the administration, animals are sacrificed, and selected tissues harvested for bioanalytical and histological evaluation. SOD1 protein and mRNA levels are assessed for suppression after treatment with pri-miRNA packaged in AAV-DJ with a CBA promoter, relative to the vehicle group.

### Example 18. SOD1 Knock-Down In Vivo Using VOYmiR-114.806

In Tg (SOD1) 3Cje/J mice, VOYmiR-114.806 packaged in AAVDJ with a CBA promoter as described in Example 17. The mice were administered by unilateral intrastriatal administration a dose of 3.7×10⁹ vg. After 1 or 2 weeks, there was no significant reduction in normalized SOD1 protein levels; normalized SOD1 protein levels were 98±11% (standard deviation) and 98±10% of the vehicle control group after 1 and 2 weeks, respectively. By week 3, VOYmiR-114.806 reduced the normalized SOD1 protein level to 84±9.0% of the vehicle control group, which was statistically significant (p<0.05, One-way ANOVA with Dunnett's post-hoc analysis). By weeks 4 and 6, VOYmiR-114.806 reduced the normalized SOD1 protein level to 73±7.9% (p<0.0001) and 75±7.4% (p<0.0001), respectively, of the vehicle control group. These results demonstrate that VOYmiR-114.806 packaged in AAV-DJ with a CBA promoter, is efficacious in vivo in down-modulating SOD1 protein levels. In addition, these results demonstrate that a total intrastriatal dose as low as 3.7×10⁹ vg of VOYmiR-114.806 packaged in AAVDJ with a CBA promoter resulted in significant down-modulation of SOD1 protein levels.

1-25. (canceled)

26. An adeno-associated viral (AAV) vector genome comprising a nucleic acid positioned between two inverted terminal repeats (ITRs), wherein the nucleic acid encodes a sense strand sequence and an antisense strand sequence of a siRNA, wherein the sense strand sequence comprises at least 15 contiguous nucleotides differing by no more than 3 nucleotides from any one of the nucleotide sequences listed in Table 3, Table 11 or Table 14 and the antisense strand sequence comprises at least 15 contiguous nucleotides differing by no more than 3 nucleotides from any one of the nucleotide sequences listed in Table 3, Table 11 or Table 14, and wherein the sense strand sequence and the antisense strand sequence share a region of complementarity of at least 17 nucleotides in length.

27. The AAV vector of claim 26, wherein:
(i) the sense strand sequence and the antisense strand sequence are, independently, 21 nucleotides in length; and/or
(ii) at least one of the sense strand sequence and the antisense strand sequence comprise a 3′ overhang of at least 1 or 2 nucleotides.

28. The AAV vector genome of claim 26, which further encodes a modulatory polynucleotide comprising the sense strand sequence and the antisense strand sequence, wherein the modulatory polynucleotide comprises:
(i) a 5′ flanking region;
(ii) a loop region; and/or
(iii) a 3′ flanking region.

29. The AAV vector genome of claim 26, which further comprises:
(i) a promoter;
(ii) an enhancer
(iii) an intron; and/or
(iv) a polyA sequence.

30. An adeno-associated virus (AAV) particle comprising the AAV vector genome of claim 26 and an AAV capsid protein.

31. The AAV particle of claim 30, wherein the AAV capsid protein is an AAV9 capsid protein or variant thereof, an AAV5 capsid protein or variant thereof, or an AAVrh10 capsid protein or variant thereof.

32. A siRNA for inhibiting expression of SOD1 comprising a sense strand sequence and an antisense strand sequence, wherein the sense strand sequence comprises at least 15 contiguous nucleotides differing by no more than 3 nucleotides from any one of the nucleotide sequence of sequences listed in Table 3, Table 11 or Table 14 and the antisense strand sequence comprises at least 15 contiguous nucleotides differing by no more than 3 nucleotides from any one of the nucleotide sequence of sequences listed in Table 3, Table 11 or Table 14, and wherein said sense strand sequence and antisense strand sequence share a region of complementarity of at least 17 nucleotides in length.

33. A modulatory polynucleotide comprising the siRNA of claim 32, wherein the modulatory polynucleotide further comprises:
(i) a 5′ flanking region;
(ii) a loop region; and/or
(iii) a 3′ flanking region.

34. A pharmaceutical composition comprising the AAV particle of claim 30, and a pharmaceutically acceptable carrier.

35. A pharmaceutical composition comprising the siRNA of claim 32, and a pharmaceutically acceptable excipient.

36. A method for inhibiting the expression of SOD1 gene in a cell comprising administering to the cell the AAV particle of claim 30.

37. A method for inhibiting the expression of SOD1 gene in a cell comprising administering to the cell the siRNA of claim 32, optionally wherein the cell is
(i) a mammalian cell;
(ii) a CNS cell;
(iii) a neuron;
(iv) a motor neuron or a ventral horn motor neuron; or
(v) an astrocyte.

38. The method of claim 37, wherein the cell is
(i) a mammalian cell;
(ii) a CNS cell;
(iii) a neuron;
(iv) a motor neuron or a ventral horn motor neuron; or
(v) an astrocyte.

39. A method for treating and/or ameliorating amyotrophic lateral sclerosis (ALS) in a subject, the method comprising administering to the subject an AAV particle comprising an AAV vector genome comprising a nucleic acid positioned between two inverted terminal repeats (ITRs), and wherein the nucleic acid sequence encodes a sense strand sequence and an antisense strand sequence of a siRNA, wherein the sense strand sequence comprises at least 15 contiguous nucleotides differing by no more than 3 nucleotides from any one of the nucleotide sequences listed in Table 3, Table 11 or Table 14 and the antisense strand sequence comprises at least 15 contiguous nucleotides differing by no more than 3 nucleotides from any one of the nucleotide sequences listed in Table 3, Table 11 or Table 14; wherein the sense strand sequence and the antisense strand sequence share a region of complementarity of at least 17 nucleotides in length; thereby treating the subject.

40. The method of claim 39, wherein the expression of SOD1 is inhibited or suppressed in a CNS cell and/or a CNS region.

41. The method of claim 40, wherein:
(i) the CNS cell comprises a motor neuron;
(ii) the CNS region comprises a spinal cord region, a forebrain region, a midbrain region, the hindbrain region, or a combination thereof; and/or
(iii) the CNS region comprises a spinal cord region.

42. The method of claim 40, wherein the SOD1 is a wild type SOD1, a mutated SOD1, or a combination thereof.

43. The method of claim 39, wherein the ALS is:
(i) familial ALS;
(ii) sporadic ALS;
(iii) early stage ALS;
(iv) middle stage ALS; and/or
(v) late stage ALS.

44. The method of claim 39, wherein the AAV particle is administered intravenously to the subject.

45. A method of treating amyotrophic lateral sclerosis (ALS) in a subject, comprising administering to the subject, the siRNA of claim 32, thereby treating the subject.


SEQUENCE LISTING (396 sequences)

SEQ ID NO: 1 - DNA, 981 bp, Homo sapiens
  source 1..981: mol_type = genomic DNA
gtttggggccagagtgggcgaggcgcggaggtctggcctataaagtagtcgcggagacggggtgctggtttgcgtcgtagtctcctgcagcgtctggggtttccgttgcagtcctcggaaccaggacctcggcgtggcctagcgagttatggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaataaacattcccttggatgtagtctgaggccccttaactcatctgttatcctgctagctgtagaaatgtatcctgataaacattaaacactgtaatcttaaaagtgtaattgtgtgactttttcagagttgctttaaagtacctgtagtgagaaactgatttatgatcacttggaagatttgtatagttttataaaactcagttaaaatgtctgtttcaatgacctgtattttgccagacttaaatcacagatgggtattaaacttgtcagaatttctttgtcattcaagcctgtgaataaaaaccctgtatggcacttattatgaggctattaaaagaatccaaattcaaactaaaaaaaaaaaaaaaaaa

SEQ ID NO: 2 - DNA, 465 bp, Macaca fascicularis
  source 1..465: mol_type = genomic DNA
atggcgatgaaggccgtgtgcgtgttgaagggcgacagcccagtgcagggcaccatcaatttcgagcagaaggaaagtaatggaccagtgaaggtgtggggaagcattacaggattgactgaaggcctgcatggattccatgttcatcagtttggagataatacacaaggctgtaccagtgcaggtcctcactttaatcctctatccagacaacacggtgggccaaaggatgaagagaggcatgttggagacctgggcaatgtgactgctggcaaagatggtgtggccaaggtgtctttcgaagattctgtgatctcgctctcaggagaccattccatcattggccgcacattggtggtccatgaaaaagcagatgacttgggcaaaggtggaaatgaagaaagtaaaaagacaggaaacgctggaggtcgtctggcttgtggtgtaattgggatcgcccaataa

SEQ ID NO: 3 - DNA, 464 bp, Macaca mulatta
  source 1..464: mol_type = genomic DNA
atggcgatgaaggccgtgtgcgtgttgaagggcgacagcccagtgcagggcaccatcaatttcgagcagaaggaaagtaatggaccagtgaaggtgtggggaagcattacaggattgactgaaggcctgcatggattccatgttcatcagtttggagataatacacaaggctgtaccagtgcaggtcctcactttaatcctctatccagacaacacggtgggccaaaggatgaagagaggcatgttggagacctgggcaatgtgactgctggcaaagatggtgtggccaaggtgtctttcgaagattctgtgatctcgctctcaggagaccattccatcattggccgcacattggtggtccatgaaaaagcagatgacttgggcaaaggtggaaatgaagaaagtaaaaagacaggaaacgctggaggtcgtctggcttgtggtgtaattgggatcgccaataa

SEQ ID NO: 4 - RNA, 21 nt, synthetic construct
  modified_base 20..21: mod_base = OTHER; note = thymine
  misc_feature 1..21: note = Synthetic
  source 1..21: mol_type = other RNA
cggaggtctggcctataactt

SEQ ID NO: 5 - RNA, 21 nt, synthetic construct
  modified_base 20..21: mod_base = OTHER; note = thymine
  misc_feature 1..21: note = Synthetic
  source 1..21: mol_type = other RNA
ggaggtctggcctataaactt

SEQ ID NO: 6 - RNA, 21 nt, synthetic construct
  modified_base 20..21: mod_base = OTHER; note = thymine
  misc_feature 1..21: note = Synthetic
  source 1..21: mol_type = other RNA
gaggtctggcctataaagctt

SEQ ID NO: 7 - RNA, 21 nt, synthetic construct
  modified_base 20..21: mod_base = OTHER; note = thymine
  misc_feature 1..21: note = Synthetic
  source 1..21: mol_type = other RNA
aggtctggcctataaagtctt

SEQ ID NO: 8 - RNA, 21 nt, synthetic construct
  modified_base 20..21: mod_base = OTHER; note = thymine
  misc_feature 1..21: note = Synthetic
  source 1..21: mol_type = other RNA
ggtctggcctataaagtactt

SEQ ID NO: 9 - RNA, 21 nt, synthetic construct
  modified_base 20..21: mod_base = OTHER; note = thymine
  misc_feature 1..21: note = Synthetic
  source 1..21: mol_type = other RNA
tctggcctataaagtagtctt

SEQ ID NO: 10 - RNA, 21 nt, synthetic construct
  modified_base 20..21: mod_base = OTHER; note = thymine
  misc_feature 1..21: note = Synthetic
  source 1..21: mol_type = other RNA
ctggcctataaagtagtcctt

SEQ ID NO: 11 - RNA, 21 nt, synthetic construct
  modified_base 20..21: mod_base = OTHER; note = thymine
  misc_feature 1..21: note = Synthetic
  source 1..21: mol_type = other RNA
tggcctataaagtagtcgctt

SEQ ID NO: 12 - RNA, 21 nt, synthetic construct
  modified_base 20..21: mod_base = OTHER; note = thymine
  misc_feature 1..21: note = Synthetic
  source 1..21: mol_type = other RNA
ggcctataaagtagtcgcctt

SEQ ID NO: 13 - RNA, 21 nt, synthetic construct
  modified_base 20..21: mod_base = OTHER; note = thymine
  misc_feature 1..21: note = Synthetic
  source 1..21: mol_type = other RNA
gcctataaagtagtcgcgctt

SEQ ID NO: 14 - RNA, 21 nt, synthetic construct
  modified_base 20..21: mod_base = OTHER; note = thymine
  misc_feature 1..21: note = Synthetic
  source 1..21: mol_type = other RNA
cctataaagtagtcgcggctt

SEQ ID NO: 15 - RNA, 21 nt, synthetic construct
  modified_base 20..21: mod_base = OTHER; note = thymine
  misc_feature 1..21: note = Synthetic
  source 1..21: mol_type = other RNA
gtcgtagtctcctgcagcctt

SEQ ID NO: 16 - RNA, 21 nt, synthetic construct
  modified_base 20..21: mod_base = OTHER; note = thymine
  misc_feature 1..21: note = Synthetic
  source 1..21: mol_type = other RNA
cgtagtctcctgcagcgtctt

SEQ ID NO: 17 - RNA, 21 nt, synthetic construct
  modified_base 20..21: mod_base = OTHER; note = thymine
  misc_feature 1..21: note = Synthetic
  source 1..21: mol_type = other RNA
gtagtctcctgcagcgtcctt

SEQ ID NO: 18 - RNA, 21 nt, synthetic construct
  modified_base 20..21: mod_base = OTHER; note = thymine
  misc_feature 1..21: note = Synthetic
  source 1..21: mol_type = other RNA
tagtctcctgcagcgtctctt

SEQ ID NO: 19 - RNA, 21 nt, synthetic construct
  modified_base 20..21: mod_base = OTHER; note = thymine
  misc_feature 1..21: note = Synthetic
  source 1..21: mol_type = other RNA
atggcgacgaaggccgtgctt

SEQ ID NO: 20 - RNA, 21 nt, synthetic construct
  modified_base 20..21: mod_base = OTHER; note = thymine
  misc_feature 1..21: note = Synthetic
  source 1..21: mol_type = other RNA
cgacgaaggccgtgtgcgctt

SEQ ID NO: 21 - RNA, 21 nt, synthetic construct
  modified_base 20..21: mod_base = OTHER; note = thymine
  misc_feature 1..21: note = Synthetic
  source 1..21: mol_type = other RNA
gaaggccgtgtgcgtgctctt

SEQ ID NO: 22 - RNA, 21 nt, synthetic construct
  modified_base 20..21: mod_base = OTHER; note = thymine
  misc_feature 1..21: note = Synthetic
  source 1..21: mol_type = other RNA
ggccgtgtgcgtgctgaactt

SEQ ID NO: 23 - RNA, 21 nt, synthetic construct
  modified_base 20..21: mod_base = OTHER; note = thymine
  misc_feature 1..21: note = Synthetic
  source 1..21: mol_type = other RNA
agggcgacggcccagtgcctt

SEQ ID NO: 24 - RNA, 21 nt, synthetic construct
  modified_base 20..21: mod_base = OTHER; note = thymine
  misc_feature 1..21: note = Synthetic
  source 1..21: mol_type = other RNA
tgcagggcatcatcaattctt

SEQ ID NO: 25 - RNA, 21 nt, synthetic construct
  modified_base 20..21: mod_base = OTHER; note = thymine
  misc_feature 1..21: note = Synthetic
  source 1..21: mol_type = other RNA
gcagggcatcatcaatttctt

SEQ ID NO: 26 - RNA, 21 nt, synthetic construct
  modified_base 20..21: mod_base = OTHER; note = thymine
  misc_feature 1..21: note = Synthetic
  source 1..21: mol_type = other RNA
agggcatcatcaatttcgctt

SEQ ID NO: 27 - RNA, 21 nt, synthetic construct
  modified_base 20..21: mod_base = OTHER; note = thymine
  misc_feature 1..21: note = Synthetic
  source 1..21: mol_type = other RNA
gggcatcatcaatttcgactt

SEQ ID NO: 28 - RNA, 21 nt, synthetic construct
  modified_base 20..21: mod_base = OTHER; note = thymine
  misc_feature 1..21: note = Synthetic
  source 1..21: mol_type = other RNA
ggcatcatcaatttcgagctt

SEQ ID NO: 29 - RNA, 21 nt, synthetic construct
  modified_base 20..21: mod_base = OTHER; note = thymine
  misc_feature 1..21: note = Synthetic
  source 1..21: mol_type = other RNA
gcatcatcaatttcgagcctt

SEQ ID NO: 30 - RNA, 21 nt, synthetic construct
  modified_base 20..21: mod_base = OTHER; note = thymine
  misc_feature 1..21: note = Synthetic
  source 1..21: mol_type = other RNA
catcatcaatttcgagcactt

SEQ ID NO: 31 - RNA, 21 nt, synthetic construct
  modified_base 20..21: mod_base = OTHER; note = thymine
  misc_feature 1..21: note = Synthetic
  source 1..21: mol_type = other RNA
aatttcgagcagaaggaactt

SEQ ID NO: 32 - RNA, 21 nt, synthetic construct
  modified_base 20..21: mod_base = OTHER; note = thymine
  misc_feature 1..21: note = Synthetic
  source 1..21: mol_type = other RNA
ttcgagcagaaggaaagtctt

SEQ ID NO: 33 - RNA, 21 nt, synthetic construct
  modified_base 20..21: mod_base = OTHER; note = thymine
  misc_feature 1..21: note = Synthetic
  source 1..21: mol_type = other RNA
tcgagcagaaggaaagtactt

SEQ ID NO: 34 - RNA, 21 nt, synthetic construct
  modified_base 20..21: mod_base = OTHER; note = thymine
  misc_feature 1..21: note = Synthetic
  source 1..21: mol_type = other RNA
aaggtgtggggaagcattctt

SEQ ID NO: 35 - RNA, 21 nt, synthetic construct
  modified_base 20..21: mod_base = OTHER; note = thymine
  misc_feature 1..21: note = Synthetic
  source 1..21: mol_type = other RNA
ggtgtggggaagcattaactt

SEQ ID NO: 36 - RNA, 21 nt, synthetic construct
  modified_base 20..21: mod_base = OTHER; note = thymine
  misc_feature 1..21: note = Synthetic
  source 1..21: mol_type = other RNA
gactgactgaaggcctgcctt

SEQ ID NO: 37 - RNA, 21 nt, synthetic construct
  modified_base 20..21: mod_base = OTHER; note = thymine
  misc_feature 1..21: note = Synthetic
  source 1..21: mol_type = other RNA
ctgactgaaggcctgcatctt

SEQ ID NO: 38 - RNA, 21 nt, synthetic construct
  modified_base 20..21: mod_base = OTHER; note = thymine
  misc_feature 1..21: note = Synthetic
  source 1..21: mol_type = other RNA
tgactgaaggcctgcatgctt

SEQ ID NO: 39 - RNA, 21 nt, synthetic construct
  modified_base 20..21: mod_base = OTHER; note = thymine
  misc_feature 1..21: note = Synthetic
  source 1..21: mol_type = other RNA
tgaaggcctgcatggattctt

SEQ ID NO: 40 - RNA, 21 nt, synthetic construct
  modified_base 20..21: mod_base = OTHER; note = thymine
  misc_feature 1..21: note = Synthetic
  source 1..21: mol_type = other RNA
gaaggcctgcatggattcctt

SEQ ID NO: 41 - RNA, 21 nt, synthetic construct
  modified_base 20..21: mod_base = OTHER; note = thymine
  misc_feature 1..21: note = Synthetic
  source 1..21: mol_type = other RNA
tgcatggattccatgttcctt

SEQ ID NO: 42 - RNA, 21 nt, synthetic construct
  modified_base 20..21: mod_base = OTHER; note = thymine
  misc_feature 1..21: note = Synthetic
  source 1..21: mol_type = other RNA
catggattccatgttcatctt

SEQ ID NO: 43 - RNA, 21 nt, synthetic construct
  modified_base 20..21: mod_base = OTHER; note = thymine
  misc_feature 1..21: note = Synthetic
  source 1..21: mol_type = other RNA
ggattccatgttcatgagctt

SEQ ID NO: 44 - RNA, 21 nt, synthetic construct
  modified_base 20..21: mod_base = OTHER; note = thymine
  misc_feature 1..21: note = Synthetic
  source 1..21: mol_type = other RNA
ttccatgttcatgagtttctt

SEQ ID NO: 45 - RNA, 21 nt, synthetic construct
  modified_base 20..21: mod_base = OTHER; note = thymine
  misc_feature 1..21: note = Synthetic
  source 1..21: mol_type = other RNA
gttcatgagtttggagatctt

SEQ ID NO: 46 - RNA, 21 nt, synthetic construct
  modified_base 20..21: mod_base = OTHER; note = thymine
  misc_feature 1..21: note = Synthetic
  source 1..21: mol_type = other RNA
ttcatgagtttggagatactt

SEQ ID NO: 47 - RNA, 21 nt, synthetic construct
  modified_base 20..21: mod_base = OTHER; note = thymine
  misc_feature 1..21: note = Synthetic
  source 1..21: mol_type = other RNA
tgagtttggagataatacctt

SEQ ID NO: 48 - RNA, 21 nt, synthetic construct
  modified_base 20..21: mod_base = OTHER; note = thymine
  misc_feature 1..21: note = Synthetic
  source 1..21: mol_type = other RNA
gagtttggagataatacactt

SEQ ID NO: 49 - RNA, 21 nt, synthetic construct
  modified_base 20..21: mod_base = OTHER; note = thymine
  misc_feature 1..21: note = Synthetic
  source 1..21: mol_type = other RNA
aggctgtaccagtgcaggctt

SEQ ID NO: 50 - RNA, 21 nt, synthetic construct
  modified_base 20..21: mod_base = OTHER; note = thymine
  misc_feature 1..21: note = Synthetic
  source 1..21: mol_type = other RNA
ggctgtaccagtgcaggtctt

SEQ ID NO: 51 - RNA, 21 nt, synthetic construct
  modified_base 20..21: mod_base = OTHER; note = thymine
  misc_feature 1..21: note = Synthetic
  source 1..21: mol_type = other RNA
gcaggtcctcactttaatctt

SEQ ID NO: 52 - RNA, 21 nt, synthetic construct
  modified_base 20..21: mod_base = OTHER; note = thymine
  misc_feature 1..21: note = Synthetic
  source 1..21: mol_type = other RNA
caggtcctcactttaatcctt

SEQ ID NO: 53 - RNA, 21 nt, synthetic construct
  modified_base 20..21: mod_base = OTHER; note = thymine
  misc_feature 1..21: note = Synthetic
  source 1..21: mol_type = other RNA
tcactttaatcctctatcctt

SEQ ID NO: 54 - RNA, 21 nt, synthetic construct
  modified_base 20..21: mod_base = OTHER; note = thymine
  misc_feature 1..21: note = Synthetic
  source 1..21: mol_type = other RNA
ctatccagaaaacacggtctt

SEQ ID NO: 55 - RNA, 21 nt, synthetic construct
  modified_base 20..21: mod_base = OTHER; note = thymine
  misc_feature 1..21: note = Synthetic
  source 1..21: mol_type = other RNA
tatccagaaaacacggtgctt

SEQ ID NO: 56 - RNA, 21 nt, synthetic construct
  modified_base 20..21: mod_base = OTHER; note = thymine
  misc_feature 1..21: note = Synthetic
  source 1..21: mol_type = other RNA
atccagaaaacacggtggctt

SEQ ID NO: 57 - RNA, 21 nt, synthetic construct
  modified_base 20..21: mod_base = OTHER; note = thymine
  misc_feature 1..21: note = Synthetic
  source 1..21: mol_type = other RNA
ccagaaaacacggtgggcctt

SEQ ID NO: 58 - RNA, 21 nt, synthetic construct
  modified_base 20..21: mod_base = OTHER; note = thymine
  misc_feature 1..21: note = Synthetic
  source 1..21: mol_type = other RNA
gaaaacacggtgggccaactt

SEQ ID NO: 59 - RNA, 21 nt, synthetic construct
  modified_base 20..21: mod_base = OTHER; note = thymine
  misc_feature 1..21: note = Synthetic
  source 1..21: mol_type = other RNA
aaaacacggtgggccaaactt

SEQ ID NO: 60 - RNA, 21 nt, synthetic construct
  modified_base 20..21: mod_base = OTHER; note = thymine
  misc_feature 1..21: note = Synthetic
  source 1..21: mol_type = other RNA
cggtgggccaaaggatgactt

SEQ ID NO: 61 - RNA, 21 nt, synthetic construct
  modified_base 20..21: mod_base = OTHER; note = thymine
  misc_feature 1..21: note = Synthetic
  source 1..21: mol_type = other RNA
aggatgaagagaggcatgctt

SEQ ID NO: 62 - RNA, 21 nt, synthetic construct
  modified_base 20..21: mod_base = OTHER; note = thymine
  misc_feature 1..21: note = Synthetic
  source 1..21: mol_type = other RNA
atgaagagaggcatgttgctt

SEQ ID NO: 63 - RNA, 21 nt, synthetic construct
  modified_base 20..21: mod_base = OTHER; note = thymine
  misc_feature 1..21: note = Synthetic
  source 1..21: mol_type = other RNA
gagaggcatgttggagacctt

SEQ ID NO: 64 - RNA, 21 nt, synthetic construct
  modified_base 20..21: mod_base = OTHER; note = thymine
  misc_feature 1..21: note = Synthetic
  source 1..21: mol_type = other RNA
agaggcatgttggagactctt

SEQ ID NO: 65 - RNA, 21 nt, synthetic construct
  modified_base 20..21: mod_base = OTHER; note = thymine
  misc_feature 1..21: note = Synthetic
  source 1..21: mol_type = other RNA
atgttggagacttgggcactt

SEQ ID NO: 66 - RNA, 21 nt, synthetic construct
  modified_base 20..21: mod_base = OTHER; note = thymine
  misc_feature 1..21: note = Synthetic
  source 1..21: mol_type = other RNA
gttggagacttgggcaatctt

SEQ ID NO: 67 - RNA, 21 nt, synthetic construct
  modified_base 20..21: mod_base = OTHER; note = thymine
  misc_feature 1..21: note = Synthetic
  source 1..21: mol_type = other RNA
ggagacttgggcaatgtgctt

SEQ ID NO: 68 - RNA, 21 nt, synthetic construct
  modified_base 20..21: mod_base = OTHER; note = thymine
  misc_feature 1..21: note = Synthetic
  source 1..21: mol_type = other RNA
ggcaatgtgactgctgacctt

SEQ ID NO: 69 - RNA, 21 nt, synthetic construct
  modified_base 20..21: mod_base = OTHER; note = thymine
  misc_feature 1..21: note = Synthetic
  source 1..21: mol_type = other RNA
caatgtgactgctgacaactt

SEQ ID NO: 70 - RNA, 21 nt, synthetic construct
  modified_base 20..21: mod_base = OTHER; note = thymine
  misc_feature 1..21: note = Synthetic
  source 1..21: mol_type = other RNA
ctgacaaagatggtgtggctt

SEQ ID NO: 71 - RNA, 21 nt, synthetic construct
  modified_base 20..21: mod_base = OTHER; note = thymine
  misc_feature 1..21: note = Synthetic
  source 1..21: mol_type = other RNA
tgacaaagatggtgtggcctt

SEQ ID NO: 72 - RNA, 21 nt, synthetic construct
  modified_base 20..21: mod_base = OTHER; note = thymine
  misc_feature 1..21: note = Synthetic
  source 1..21: mol_type = other RNA
ctcaggagaccattgcatctt

SEQ ID NO: 73 - RNA, 21 nt, synthetic construct
  modified_base 20..21: mod_base = OTHER; note = thymine
  misc_feature 1..21: note = Synthetic
  source 1..21: mol_type = other RNA
tcaggagaccattgcatcctt

SEQ ID NO: 74 - RNA, 21 nt, synthetic construct
  modified_base 20..21: mod_base = OTHER; note = thymine
  misc_feature 1..21: note = Synthetic
  source 1..21: mol_type = other RNA
agaccattgcatcattggctt

SEQ ID NO: 75 - RNA, 21 nt, synthetic construct
  modified_base 20..21: mod_base = OTHER; note = thymine
  misc_feature 1..21: note = Synthetic
  source 1..21: mol_type = other RNA
gaccattgcatcattggcctt

SEQ ID NO: 76 - RNA, 21 nt, synthetic construct
  modified_base 20..21: mod_base = OTHER; note = thymine
  misc_feature 1..21: note = Synthetic
  source 1..21: mol_type = other RNA
attgcatcattggccgcactt

SEQ ID NO: 77 - RNA, 21 nt, synthetic construct
  modified_base 20..21: mod_base = OTHER; note = thymine
  misc_feature 1..21: note = Synthetic
  source 1..21: mol_type = other RNA
cattggccgcacactggtctt

SEQ ID NO: 78 - RNA, 21 nt, synthetic construct
  modified_base 20..21: mod_base = OTHER; note = thymine
  misc_feature 1..21: note = Synthetic
  source 1..21: mol_type = other RNA
cgcacactggtggtccatctt

SEQ ID NO: 79 - RNA, 21 nt, synthetic construct
  modified_base 20..21: mod_base = OTHER; note = thymine
  misc_feature 1..21: note = Synthetic
  source 1..21: mol_type = other RNA
cacactggtggtccatgactt

SEQ ID NO: 80 - RNA, 21 nt, synthetic construct
  modified_base 20..21: mod_base = OTHER; note = thymine
  misc_feature 1..21: note = Synthetic
  source 1..21: mol_type = other RNA
acactggtggtccatgaactt

SEQ ID NO: 81 - RNA, 21 nt, synthetic construct
  modified_base 20..21: mod_base = OTHER; note = thymine
  misc_feature 1..21: note = Synthetic
  source 1..21: mol_type = other RNA
tggtggtccatgaaaaagctt

SEQ ID NO: 82 - RNA, 21 nt, synthetic construct
  modified_base 20..21: mod_base = OTHER; note = thymine
  misc_feature 1..21: note = Synthetic
  source 1..21: mol_type = other RNA
tggtccatgaaaaagcagctt

SEQ ID NO: 83 - RNA, 21 nt, synthetic construct
  modified_base 20..21: mod_base = OTHER; note = thymine
  misc_feature 1..21: note = Synthetic
  source 1..21: mol_type = other RNA
aaagcagatgacttgggcctt

SEQ ID NO: 84 - RNA, 21 nt, synthetic construct
  modified_base 20..21: mod_base = OTHER; note = thymine
  misc_feature 1..21: note = Synthetic
  source 1..21: mol_type = other RNA
gcagatgacttgggcaaactt

SEQ ID NO: 85 - RNA, 21 nt, synthetic construct
  modified_base 20..21: mod_base = OTHER; note = thymine
  misc_feature 1..21: note = Synthetic
  source 1..21: mol_type = other RNA
atgacttgggcaaaggtgctt

SEQ ID NO: 86 - RNA, 21 nt, synthetic construct
  modified_base 20..21: mod_base = OTHER; note = thymine
  misc_feature 1..21: note = Synthetic
  source 1..21: mol_type = other RNA
tgacttgggcaaaggtggctt

SEQ ID NO: 87 - RNA, 21 nt, synthetic construct
  modified_base 20..21: mod_base = OTHER; note = thymine
  misc_feature 1..21: note = Synthetic
  source 1..21: mol_type = other RNA
gacttgggcaaaggtggactt

SEQ ID NO: 88 - RNA, 21 nt, synthetic construct
  modified_base 20..21: mod_base = OTHER; note = thymine
  misc_feature 1..21: note = Synthetic
  source 1..21: mol_type = other RNA
gtacaaagacaggaaacgctt

SEQ ID NO: 89 - RNA, 21 nt, synthetic construct
  modified_base 20..21: mod_base = OTHER; note = thymine
  misc_feature 1..21: note = Synthetic
  source 1..21: mol_type = other RNA
acaaagacaggaaacgctctt

SEQ ID NO: 90 - RNA, 21 nt, synthetic construct
  modified_base 20..21: mod_base = OTHER; note = thymine
  misc_feature 1..21: note = Synthetic
  source 1..21: mol_type = other RNA
caaagacaggaaacgctgctt

SEQ ID NO: 91 - RNA, 21 nt, synthetic construct
  modified_base 20..21: mod_base = OTHER; note = thymine
  misc_feature 1..21: note = Synthetic
  source 1..21: mol_type = other RNA
aggaaacgctggaagtcgctt

SEQ ID NO: 92 - RNA, 21 nt, synthetic construct
  modified_base 20..21: mod_base = OTHER; note = thymine
  misc_feature 1..21: note = Synthetic
  source 1..21: mol_type = other RNA
gtcgtttggcttgtggtgctt

SEQ ID NO: 93 - RNA, 21 nt, synthetic construct
  modified_base 20..21: mod_base = OTHER; note = thymine
  misc_feature 1..21: note = Synthetic
  source 1..21: mol_type = other RNA
tcgtttggcttgtggtgtctt

SEQ ID NO: 94 - RNA, 21 nt, synthetic construct
  modified_base 20..21: mod_base = OTHER; note = thymine
  misc_feature 1..21: note = Synthetic
  source 1..21: mol_type = other RNA
cgtttggcttgtggtgtactt

SEQ ID NO: 95 - RNA, 21 nt, synthetic construct
  modified_base 20..21: mod_base = OTHER; note = thymine
  misc_feature 1..21: note = Synthetic
  source 1..21: mol_type = other RNA
gtttggcttgtggtgtaactt

SEQ ID NO: 96 - RNA, 21 nt, synthetic construct
  modified_base 20..21: mod_base = OTHER; note = thymine
  misc_feature 1..21: note = Synthetic
  source 1..21: mol_type = other RNA
ttggcttgtggtgtaattctt

SEQ ID NO: 97 - RNA, 21 nt, synthetic construct
  modified_base 20..21: mod_base = OTHER; note = thymine
  misc_feature 1..21: note = Synthetic
  source 1..21: mol_type = other RNA
ggcttgtggtgtaattggctt

SEQ ID NO: 98 - RNA, 21 nt, synthetic construct
  modified_base 20..21: mod_base = OTHER; note = thymine
  misc_feature 1..21: note = Synthetic
  source 1..21: mol_type = other RNA
gcttgtggtgtaattgggctt

SEQ ID NO: 99 - RNA, 21 nt, synthetic construct
  modified_base 20..21: mod_base = OTHER; note = thymine
  misc_feature 1..21: note = Synthetic
  source 1..21: mol_type = other RNA
cttgtggtgtaattgggactt

SEQ ID NO: 100 - RNA, 21 nt, synthetic construct
  modified_base 20..21: mod_base = OTHER; note = thymine
  misc_feature 1..21: note = Synthetic
  source 1..21: mol_type = other RNA
tgtggtgtaattgggatcctt

SEQ ID NO: 101 - RNA, 21 nt, synthetic construct
  modified_base 20..21: mod_base = OTHER; note = thymine
  misc_feature 1..21: note = Synthetic
  source 1..21: mol_type = other RNA
gtggtgtaattgggatcgctt

SEQ ID NO: 102 - RNA, 21 nt, synthetic construct
  modified_base 20..21: mod_base = OTHER; note = thymine
  misc_feature 1..21: note = Synthetic
  source 1..21: mol_type = other RNA
tggtgtaattgggatcgcctt

SEQ ID NO: 103 - RNA, 21 nt, synthetic construct
  modified_base 20..21: mod_base = OTHER; note = thymine
  misc_feature 1..21: note = Synthetic
  source 1..21: mol_type = other RNA
gtaattgggatcgcccaactt

SEQ ID NO: 104 - RNA, 21 nt, synthetic construct
  modified_base 20..21: mod_base = OTHER; note = thymine
  misc_feature 1..21: note = Synthetic
  source 1..21: mol_type = other RNA
taattgggatcgcccaatctt

SEQ ID NO: 105 - RNA, 21 nt, synthetic construct
  modified_base 20..21: mod_base = OTHER; note = thymine
  misc_feature 1..21: note = Synthetic
  source 1..21: mol_type = other RNA
aattgggatcgcccaatactt

SEQ ID NO: 106 - RNA, 21 nt, synthetic construct
  modified_base 20..21: mod_base = OTHER; note = thymine
  misc_feature 1..21: note = Synthetic
  source 1..21: mol_type = other RNA
attgggatcgcccaataactt

SEQ ID NO: 107 - RNA, 21 nt, synthetic construct
  modified_base 20..21: mod_base = OTHER; note = thymine
  misc_feature 1..21: note = Synthetic
  source 1..21: mol_type = other RNA
ttgggatcgcccaataaactt

SEQ ID NO: 108 - RNA, 21 nt, synthetic construct
  modified_base 20..21: mod_base = OTHER; note = thymine
  misc_feature 1..21: note = Synthetic
  source 1..21: mol_type = other RNA
tgggatcgcccaataaacctt

SEQ ID NO: 109 - RNA, 21 nt, synthetic construct
  modified_base 20..21: mod_base = OTHER; note = thymine
  misc_feature 1..21: note = Synthetic
  source 1..21: mol_type = other RNA
gggatcgcccaataaacactt

SEQ ID NO: 110 - RNA, 21 nt, synthetic construct
  modified_base 20..21: mod_base = OTHER; note = thymine
  misc_feature 1..21: note = Synthetic
  source 1..21: mol_type = other RNA
atcgcccaataaacattcctt

SEQ ID NO: 111 - RNA, 21 nt, synthetic construct
  modified_base 20..21: mod_base = OTHER; note = thymine
  misc_feature 1..21: note = Synthetic
  source 1..21: mol_type = other RNA
ccaataaacattcccttgctt

SEQ ID NO: 112 - RNA, 21 nt, synthetic construct
  modified_base 20..21: mod_base = OTHER; note = thymine
  misc_feature 1..21: note = Synthetic
  source 1..21: mol_type = other RNA
caataaacattcccttggctt

SEQ ID NO: 113 - RNA, 21 nt, synthetic construct
  modified_base 20..21: mod_base = OTHER; note = thymine
  misc_feature 1..21: note = Synthetic
  source 1..21: mol_type = other RNA
aataaacattcccttggactt

SEQ ID NO: 114 - RNA, 21 nt, synthetic construct
  modified_base 20..21: mod_base = OTHER; note = thymine
  misc_feature 1..21: note = Synthetic
  source 1..21: mol_type = other RNA
ataaacattcccttggatctt

SEQ ID NO: 115 - RNA, 21 nt, synthetic construct
  modified_base 20..21: mod_base = OTHER; note = thymine
  misc_feature 1..21: note = Synthetic
  source 1..21: mol_type = other RNA
taaacattcccttggatgctt

SEQ ID NO: 116 - RNA, 21 nt, synthetic construct
  modified_base 20..21: mod_base = OTHER; note = thymine
  misc_feature 1..21: note = Synthetic
  source 1..21: mol_type = other RNA
aaacattcccttggatgtctt

SEQ ID NO: 117 - RNA, 21 nt, synthetic construct
  modified_base 20..21: mod_base = OTHER; note = thymine
  misc_feature 1..21: note = Synthetic
  source 1..21: mol_type = other RNA
aacattcccttggatgtactt

SEQ ID NO: 118 - RNA, 21 nt, synthetic construct
  modified_base 20..21: mod_base = OTHER; note = thymine
  misc_feature 1..21: note = Synthetic
  source 1..21: mol_type = other RNA
attcccttggatgtagtcctt

SEQ ID NO: 119 - RNA, 21 nt, synthetic construct
  modified_base 20..21: mod_base = OTHER; note = thymine
  misc_feature 1..21: note = Synthetic
  source 1..21: mol_type = other RNA
cttggatgtagtctgaggctt

SEQ ID NO: 120 - RNA, 21 nt, synthetic construct
  modified_base 20..21: mod_base = OTHER; note = thymine
  misc_feature 1..21: note = Synthetic
  source 1..21: mol_type = other RNA
ctgaggccccttaactcactt

SEQ ID NO: 121 - RNA, 21 nt, synthetic construct
  modified_base 20..21: mod_base = OTHER; note = thymine
  misc_feature 1..21: note = Synthetic
  source 1..21: mol_type = other RNA
gaggccccttaactcatcctt

SEQ ID NO: 122 - RNA, 21 nt, synthetic construct
  modified_base 20..21: mod_base = OTHER; note = thymine
  misc_feature 1..21: note = Synthetic
  source 1..21: mol_type = other RNA
aggccccttaactcatctctt

SEQ ID NO: 123 - RNA, 21 nt, synthetic construct
  modified_base 20..21: mod_base = OTHER; note = thymine
  misc_feature 1..21: note = Synthetic
  source 1..21: mol_type = other RNA
ccccttaactcatctgttctt

SEQ ID NO: 124 - RNA, 21 nt, synthetic construct
  modified_base 20..21: mod_base = OTHER; note = thymine
  misc_feature 1..21: note = Synthetic
  source 1..21: mol_type = other RNA
cccttaactcatctgttactt

SEQ ID NO: 125 - RNA, 21 nt, synthetic construct
  modified_base 20..21: mod_base = OTHER; note = thymine
  misc_feature 1..21: note = Synthetic
  source 1..21: mol_type = other RNA
ccttaactcatctgttatctt

SEQ ID NO: 126 - RNA, 21 nt, synthetic construct
  modified_base 20..21: mod_base = OTHER; note = thymine
  misc_feature 1..21: note = Synthetic
  source 1..21: mol_type = other RNA
cttaactcatctgttatcctt

SEQ ID NO: 127 - RNA, 21 nt, synthetic construct
  modified_base 20..21: mod_base = OTHER; note = thymine
  misc_feature 1..21: note = Synthetic
  source 1..21: mol_type = other RNA
ttaactcatctgttatccctt

SEQ ID NO: 128 - RNA, 21 nt, synthetic construct
  modified_base 20..21: mod_base = OTHER; note = thymine
  misc_feature 1..21: note = Synthetic
  source 1..21: mol_type = other RNA
taactcatctgttatcctctt

SEQ ID NO: 129 - RNA, 21 nt, synthetic construct
  modified_base 20..21: mod_base = OTHER; note = thymine
  misc_feature 1..21: note = Synthetic
  source 1..21: mol_type = other RNA
aactcatctgttatcctgctt

SEQ ID NO: 130 - RNA, 21 nt, synthetic construct
  modified_base 20..21: mod_base = OTHER; note = thymine
  misc_feature 1..21: note = Synthetic
  source 1..21: mol_type = other RNA
gttatcctgctagctgtactt

SEQ ID NO: 131 - RNA, 21 nt, synthetic construct
  modified_base 20..21: mod_base = OTHER; note = thymine
  misc_feature 1..21: note = Synthetic
  source 1..21: mol_type = other RNA
ctgctagctgtagaaatgctt

SEQ ID NO: 132 - RNA, 21 nt, synthetic construct
  modified_base 20..21: mod_base = OTHER; note = thymine
  misc_feature 1..21: note = Synthetic
  source 1..21: mol_type = other RNA
tgctagctgtagaaatgtctt

SEQ ID NO: 133 - RNA, 21 nt, synthetic construct
  modified_base 20..21: mod_base = OTHER; note = thymine
  misc_feature 1..21: note = Synthetic
  source 1..21: mol_type = other RNA
gctgtagaaatgtatcctctt

SEQ ID NO: 134 - RNA, 21 nt, synthetic construct
  modified_base 20..21: mod_base = OTHER; note = thymine
  misc_feature 1..21: note = Synthetic
  source 1..21: mol_type = other RNA
ctgtagaaatgtatcctgctt

SEQ ID NO: 135 - RNA, 21 nt, synthetic construct
  modified_base 20..21: mod_base = OTHER; note = thymine
  misc_feature 1..21: note = Synthetic
  source 1..21: mol_type = other RNA
tgtagaaatgtatcctgactt

SEQ ID NO: 136 - RNA, 21 nt, synthetic construct
  modified_base 20..21: mod_base = OTHER; note = thymine
  misc_feature 1..21: note = Synthetic
  source 1..21: mol_type = other RNA
gtagaaatgtatcctgatctt

SEQ ID NO: 137 - RNA, 21 nt, synthetic construct
  modified_base 20..21: mod_base = OTHER; note = thymine
  misc_feature 1..21: note = Synthetic
  source 1..21: mol_type = other RNA
aaatgtatcctgataaacctt

SEQ ID NO: 138 - RNA, 21 nt, synthetic construct
  modified_base 20..21: mod_base = OTHER; note = thymine
  misc_feature 1..21: note = Synthetic
  source 1..21: mol_type = other RNA
gtatcctgataaacattactt

SEQ ID NO: 139 - RNA, 21 nt, synthetic construct
  modified_base 20..21: mod_base = OTHER; note = thymine
  misc_feature 1..21: note = Synthetic
  source 1..21: mol_type = other RNA
ttaaacactgtaatcttactt

SEQ ID NO: 140 - RNA, 21 nt, synthetic construct
  modified_base 20..21: mod_base = OTHER; note = thymine
  misc_feature 1..21: note = Synthetic
  source 1..21: mol_type = other RNA
actgtaatcttaaaagtgctt

SEQ ID NO: 141 - RNA, 21 nt, synthetic construct
  modified_base 20..21: mod_base = OTHER; note = thymine
  misc_feature 1..21: note = Synthetic
  source 1..21: mol_type = other RNA
ctgtaatcttaaaagtgtctt

SEQ ID NO: 142 - RNA, 21 nt, synthetic construct
  modified_base 20..21: mod_base = OTHER; note = thymine
  misc_feature 1..21: note = Synthetic
  source 1..21: mol_type = other RNA
tgtaatcttaaaagtgtactt

SEQ ID NO: 143 - RNA, 21 nt, synthetic construct
  modified_base 20..21: mod_base = OTHER; note = thymine
  misc_feature 1..21: note = Synthetic
  source 1..21: mol_type = other RNA
gtaatcttaaaagtgtaactt

SEQ ID NO: 144 - RNA, 21 nt, synthetic construct
  modified_base 20..21: mod_base = OTHER; note = thymine
  misc_feature 1..21: note = Synthetic
  source 1..21: mol_type = other RNA
cttaaaagtgtaattgtgctt

SEQ ID NO: 145 - RNA, 21 nt, synthetic construct
  modified_base 20..21: mod_base = OTHER; note = thymine
  misc_feature 1..21: note = Synthetic
  source 1..21: mol_type = other RNA
tacctgtagtgagaaactctt

SEQ ID NO: 146 - RNA, 21 nt, synthetic construct
  modified_base 20..21: mod_base = OTHER; note = thymine
  misc_feature 1..21: note = Synthetic
  source 1..21: mol_type = other RNA
ttatgatcacttggaagactt

SEQ ID NO: 147 - RNA, 21 nt, synthetic construct
  modified_base 20..21: mod_base = OTHER; note = thymine
  misc_feature 1..21: note = Synthetic
  source 1..21: mol_type = other RNA
atgatcacttggaagattctt

SEQ ID NO: 148 - RNA, 21 nt, synthetic construct
  modified_base 20..21: mod_base = OTHER; note = thymine
  misc_feature 1..21: note = Synthetic
  source 1..21: mol_type = other RNA
atcacttggaagatttgtctt

SEQ ID NO: 149 - RNA, 21 nt, synthetic construct
  modified_base 20..21: mod_base = OTHER; note = thymine
  misc_feature 1..21: note = Synthetic
  source 1..21: mol_type = other RNA
tggaagatttgtatagttctt

SEQ ID NO: 150 - RNA, 21 nt, synthetic construct
  modified_base 20..21: mod_base = OTHER; note = thymine
  misc_feature 1..21: note = Synthetic
  source 1..21: mol_type = other RNA
tataaaactcagttaaaactt

SEQ ID NO: 151 - RNA, 21 nt, synthetic construct
  modified_base 20..21: mod_base = OTHER; note = thymine
  misc_feature 1..21: note = Synthetic
  source 1..21: mol_type = other RNA
aaactcagttaaaatgtcctt

SEQ ID NO: 152 - RNA, 21 nt, synthetic construct
  modified_base 20..21: mod_base = OTHER; note = thymine
  misc_feature 1..21: note = Synthetic
  source 1..21: mol_type = other RNA
gtctgtttcaatgacctgctt

SEQ ID NO: 153 - RNA, 21 nt, synthetic construct
  modified_base 20..21: mod_base = OTHER; note = thymine
  misc_feature 1..21: note = Synthetic
  source 1..21: mol_type = other RNA
atgacctgtattttgccactt

SEQ ID NO: 154 - RNA, 21 nt, synthetic construct
  modified_base 20..21: mod_base = OTHER; note = thymine
  misc_feature 1..21: note = Synthetic
  source 1..21: mol_type = other RNA
acctgtattttgccagacctt

SEQ ID NO: 155 - RNA, 21 nt, synthetic construct
  modified_base 20..21: mod_base = OTHER; note = thymine
  misc_feature 1..21: note = Synthetic
  source 1..21: mol_type = other RNA
cctgtattttgccagactctt

SEQ ID NO: 156 - RNA, 21 nt, synthetic construct
  modified_base 20..21: mod_base = OTHER; note = thymine
  misc_feature 1..21: note = Synthetic
  source 1..21: mol_type = other RNA
taaatcacagatgggtatctt

SEQ ID NO: 157 - RNA, 21 nt, synthetic construct
  modified_base 20..21: mod_base = OTHER; note = thymine
  misc_feature 1..21: note = Synthetic
  source 1..21: mol_type = other RNA
atcacagatgggtattaactt

SEQ ID NO: 158 - RNA, 21 nt, synthetic construct
  modified_base 20..21: mod_base = OTHER; note = thymine
  misc_feature 1..21: note = Synthetic
  source 1..21: mol_type = other RNA
tcacagatgggtattaaactt

SEQ ID NO: 159 - RNA, 21 nt, synthetic construct
  modified_base 20..21: mod_base = OTHER; note = thymine
  misc_feature 1..21: note = Synthetic
  source 1..21: mol_type = other RNA
acagatgggtattaaactctt

SEQ ID NO: 160 - RNA, 21 nt, synthetic construct
  modified_base 20..21: mod_base = OTHER; note = thymine
  misc_feature 1..21: note = Synthetic
  source 1..21: mol_type = other RNA
cagatgggtattaaacttctt

SEQ ID NO: 161 - RNA, 21 nt, synthetic construct
  modified_base 20..21: mod_base = OTHER; note = thymine
  misc_feature 1..21: note = Synthetic
  source 1..21: mol_type = other RNA
agatgggtattaaacttgctt

SEQ ID NO: 162 - RNA, 21 nt, synthetic construct
  modified_base 20..21: mod_base = OTHER; note = thymine
  misc_feature 1..21: note = Synthetic
  source 1..21: mol_type = other RNA
atgggtattaaacttgtcctt

SEQ ID NO: 163 - RNA, 21 nt, synthetic construct
  modified_base 20..21: mod_base = OTHER; note = thymine
  misc_feature 1..21: note = Synthetic
  source 1..21: mol_type = other RNA
taaacttgtcagaatttcctt

SEQ ID NO: 164 - RNA, 21 nt, synthetic construct
  modified_base 20..21: mod_base = OTHER; note = thymine
  misc_feature 1..21: note = Synthetic
  source 1..21: mol_type = other RNA
tcattcaagcctgtgaatctt

SEQ ID NO: 165 - RNA, 21 nt, synthetic construct
  modified_base 20..21: mod_base = OTHER; note = thymine
  misc_feature 1..21: note = Synthetic
  source 1..21: mol_type = other RNA
cattcaagcctgtgaatactt

SEQ ID NO: 166 - RNA, 21 nt, synthetic construct
  modified_base 20..21: mod_base = OTHER; note = thymine
  misc_feature 1..21: note = Synthetic
  source 1..21: mol_type = other RNA
aataaaaaccctgtatggctt

SEQ ID NO: 167 - RNA, 21 nt, synthetic construct
  modified_base 20..21: mod_base = OTHER; note = thymine
  misc_feature 1..21: note = Synthetic
  source 1..21: mol_type = other RNA
ataaaaaccctgtatggcctt

SEQ ID NO: 168 - RNA, 21 nt, synthetic construct
  modified_base 20..21: mod_base = OTHER; note = thymine
  misc_feature 1..21: note = Synthetic
  source 1..21: mol_type = other RNA
aaccctgtatggcacttactt

SEQ ID NO: 169 - RNA, 21 nt, synthetic construct
  modified_base 20..21: mod_base = OTHER; note = thymine
  misc_feature 1..21: note = Synthetic
  source 1..21: mol_type = other RNA
accctgtatggcacttatctt

SEQ ID NO: 170 - RNA, 21 nt, synthetic construct
  modified_base 20..21: mod_base = OTHER; note = thymine
  misc_feature 1..21: note = Synthetic
  source 1..21: mol_type = other RNA
gaggctattaaaagaatcctt

SEQ ID NO: 171 - RNA, 21 nt, synthetic construct
  modified_base 20..21: mod_base = OTHER; note = thymine
  misc_feature 1..21: note = Synthetic
  source 1..21: mol_type = other RNA
aaagaatccaaattcaaactt

SEQ ID NO: 172 - RNA, 21 nt, synthetic construct
  modified_base 20..21: mod_base = OTHER; note = thymine
  misc_feature 1..21: note = Synthetic
  source 1..21: mol_type = other RNA
gaatccaaattcaaactactt

SEQ ID NO: 173 - RNA, 21 nt, synthetic construct
  modified_base 20..21: mod_base = OTHER; note = thymine
  misc_feature 1..21: note = Synthetic
  source 1..21: mol_type = other RNA
tttataggccagacctccgtt

SEQ ID NO: 174 - RNA, 21 nt, synthetic construct
  modified_base 20..21: mod_base = OTHER; note = thymine
  misc_feature 1..21: note = Synthetic
  source 1..21: mol_type = other RNA
ttttataggccagacctcctt

SEQ ID NO: 175 - RNA, 21 nt, synthetic construct
  modified_base 20..21: mod_base = OTHER; note = thymine
  misc_feature 1..21: note = Synthetic
  source 1..21: mol_type = other RNA
tctttataggccagacctctt

SEQ ID NO: 176 - RNA, 21 nt, synthetic construct
  modified_base 20..21: mod_base = OTHER; note = thymine
  misc_feature 1..21: note = Synthetic
  source 1..21: mol_type = other RNA
tactttataggccagaccttt

SEQ ID NO: 177 - RNA, 21 nt, synthetic construct
  modified_base 20..21: mod_base = OTHER; note = thymine
  misc_feature 1..21: note = Synthetic
  source 1..21: mol_type = other RNA
ttactttataggccagacctt

SEQ ID NO: 178 - RNA, 21 nt, synthetic construct
  modified_base 20..21: mod_base = OTHER; note = thymine
  misc_feature 1..21: note = Synthetic
  source 1..21: mol_type = other RNA
tactactttataggccagatt

SEQ ID NO: 179 - RNA, 21 nt, synthetic construct
  modified_base 20..21: mod_base = OTHER; note = thymine
  misc_feature 1..21: note = Synthetic
  source 1..21: mol_type = other RNA
tgactactttataggccagtt

SEQ ID NO: 180 - RNA, 21 nt, synthetic construct
  modified_base 20..21: mod_base = OTHER; note = thymine
  misc_feature 1..21: note = Synthetic
  source 1..21: mol_type = other RNA
tcgactactttataggccatt

SEQ ID NO: 181 - RNA, 21 nt, synthetic construct
  modified_base 20..21: mod_base = OTHER; note = thymine
  misc_feature 1..21: note = Synthetic
  source 1..21: mol_type = other RNA
tgcgactactttataggcctt

SEQ ID NO: 182 - RNA, 21 nt, synthetic construct
  modified_base 20..21: mod_base = OTHER; note = thymine
  misc_feature 1..21: note = Synthetic
  source 1..21: mol_type = other RNA
tcgcgactactttataggctt

SEQ ID NO: 183 - RNA, 21 nt, synthetic construct
  modified_base 20..21: mod_base = OTHER; note = thymine
  misc_feature 1..21: note = Synthetic
  source 1..21: mol_type = other RNA
tccgcgactactttataggtt

SEQ ID NO: 184 - RNA, 21 nt, synthetic construct
  modified_base 20..21: mod_base = OTHER; note = thymine
  misc_feature 1..21: note = Synthetic
  source 1..21: mol_type = other RNA
tgctgcaggagactacgactt

SEQ ID NO: 185 - RNA, 21 nt, synthetic construct
  modified_base 20..21: mod_base = OTHER; note = thymine
  misc_feature 1..21: note = Synthetic
  source 1..21: mol_type = other RNA
tacgctgcaggagactacgtt

SEQ ID NO: 186 - RNA, 21 nt, synthetic construct
  modified_base 20..21: mod_base = OTHER; note = thymine
  misc_feature 1..21: note = Synthetic
  source 1..21: mol_type = other RNA
tgacgctgcaggagactactt

SEQ ID NO: 187 - RNA, 21 nt, synthetic construct
  modified_base 20..21: mod_base = OTHER; note = thymine
  misc_feature 1..21: note = Synthetic
  source 1..21: mol_type = other RNA
tagacgctgcaggagactatt

SEQ ID NO: 188 - RNA, 21 nt, synthetic construct
  modified_base 20..21: mod_base = OTHER; note = thymine
  misc_feature 1..21: note = Synthetic
  source 1..21: mol_type = other RNA
tcacggccttcgtcgccattt

SEQ ID NO: 189 - RNA, 21 nt, synthetic construct
  modified_base 20..21: mod_base = OTHER; note = thymine
  misc_feature 1..21: note = Synthetic
  source 1..21: mol_type = other RNA
tcgcacacggccttcgtcgtt

SEQ ID NO: 190 - RNA, 21 nt, synthetic construct
  modified_base 20..21: mod_base = OTHER; note = thymine
  misc_feature 1..21: note = Synthetic
  source 1..21: mol_type = other RNA
tagcacgcacacggccttctt

SEQ ID NO: 191 - RNA, 21 nt, synthetic construct
  modified_base 20..21: mod_base = OTHER; note = thymine
  misc_feature 1..21: note = Synthetic
  source 1..21: mol_type = other RNA
tttcagcacgcacacggcctt

SEQ ID NO: 192 - RNA, 21 nt, synthetic construct
  modified_base 20..21: mod_base = OTHER; note = thymine
  misc_feature 1..21: note = Synthetic
  source 1..21: mol_type = other RNA
tgcactgggccgtcgcccttt

SEQ ID NO: 193 - RNA, 21 nt, synthetic construct
  modified_base 20..21: mod_base = OTHER; note = thymine
  misc_feature 1..21: note = Synthetic
  source 1..21: mol_type = other RNA
taattgatgatgccctgcatt

SEQ ID NO: 194 - RNA, 21 nt, synthetic construct
  modified_base 20..21: mod_base = OTHER; note = thymine
  misc_feature 1..21: note = Synthetic
  source 1..21: mol_type = other RNA
taaattgatgatgccctgctt

SEQ ID NO: 195 - RNA, 21 nt, synthetic construct
  modified_base 20..21: mod_base = OTHER; note = thymine
  misc_feature 1..21: note = Synthetic
  source 1..21: mol_type = other RNA
tcgaaattgatgatgcccttt

SEQ ID NO: 196 - RNA, 21 nt, synthetic construct
  modified_base 20..21: mod_base = OTHER; note = thymine
  misc_feature 1..21: note = Synthetic
  source 1..21: mol_type = other RNA
ttcgaaattgatgatgccctt

SEQ ID NO: 197 - RNA, 21 nt, synthetic construct
  modified_base 20..21: mod_base = OTHER; note = thymine
  misc_feature 1..21: note = Synthetic
  source 1..21: mol_type = other RNA
tctcgaaattgatgatgcctt

SEQ ID NO: 198 - RNA, 21 nt, synthetic construct
  modified_base 20..21: mod_base = OTHER; note = thymine
  misc_feature 1..21: note = Synthetic
  source 1..21: mol_type = other RNA
tgctcgaaattgatgatgctt

SEQ ID NO: 199 - RNA, 21 nt, synthetic construct
  modified_base 20..21: mod_base = OTHER; note = thymine
  misc_feature 1..21: note = Synthetic
  source 1..21: mol_type = other RNA
ttgctcgaaattgatgatgtt

SEQ ID NO: 200 - RNA, 21 nt, synthetic construct
  modified_base 20..21: mod_base = OTHER; note = thymine
  misc_feature 1..21: note = Synthetic
  source 1..21: mol_type = other RNA
tttccttctgctcgaaatttt

SEQ ID NO: 201 - RNA, 21 nt, synthetic construct
  modified_base 20..21: mod_base = OTHER; note = thymine
  misc_feature 1..21: note = Synthetic
  source 1..21: mol_type = other RNA
tactttccttctgctcgaatt

SEQ ID NO: 202 - RNA, 21 nt, synthetic construct
  modified_base 20..21: mod_base = OTHER; note = thymine
  misc_feature 1..21: note = Synthetic
  source 1..21: mol_type = other RNA
ttactttccttctgctcgatt

SEQ ID NO: 203 - RNA, 21 nt, synthetic construct
  modified_base 20..21: mod_base = OTHER; note = thymine
  misc_feature 1..21: note = Synthetic
  source 1..21: mol_type = other RNA
taatgcttccccacacctttt

SEQ ID NO: 204 - RNA, 21 nt, synthetic construct
  modified_base 20..21: mod_base = OTHER; note = thymine
  misc_feature 1..21: note = Synthetic
  source 1..21: mol_type = other RNA
tttaatgcttccccacacctt

SEQ ID NO: 205 - RNA, 21 nt, synthetic construct
  modified_base 20..21: mod_base = OTHER; note = thymine
  misc_feature 1..21: note = Synthetic
  source 1..21: mol_type = other RNA
tgcaggccttcagtcagtctt

SEQ ID NO: 206 - RNA, 21 nt, synthetic construct
  modified_base 20..21: mod_base = OTHER; note = thymine
  misc_feature 1..21: note = Synthetic
  source 1..21: mol_type = other RNA
tatgcaggccttcagtcagtt

SEQ ID NO: 207 - RNA, 21 nt, synthetic construct
  modified_base 20..21: mod_base = OTHER; note = thymine
  misc_feature 1..21: note = Synthetic
  source 1..21: mol_type = other RNA
tcatgcaggccttcagtcatt

SEQ ID NO: 208 - RNA, 21 nt, synthetic construct
  modified_base 20..21: mod_base = OTHER; note = thymine
  misc_feature 1..21: note = Synthetic
  source 1..21: mol_type = other RNA
taatccatgcaggccttcatt

SEQ ID NO: 209 - RNA, 21 nt, synthetic construct
  modified_base 20..21: mod_base = OTHER; note = thymine
  misc_feature 1..21: note = Synthetic
  source 1..21: mol_type = other RNA
tgaatccatgcaggccttctt

SEQ ID NO: 210 - RNA, 21 nt, synthetic construct
  modified_base 20..21: mod_base = OTHER; note = thymine
  misc_feature 1..21: note = Synthetic
  source 1..21: mol_type = other RNA
tgaacatggaatccatgcatt

SEQ ID NO: 211 - RNA, 21 nt, synthetic construct
  modified_base 20..21: mod_base = OTHER; note = thymine
  misc_feature 1..21: note = Synthetic
  source 1..21: mol_type = other RNA
tatgaacatggaatccatgtt

SEQ ID NO: 212 - RNA, 21 nt, synthetic construct
  modified_base 20..21: mod_base = OTHER; note = thymine
  misc_feature 1..21: note = Synthetic
  source 1..21: mol_type = other RNA
tctcatgaacatggaatcctt

SEQ ID NO: 213 - RNA, 21 nt, synthetic construct
  modified_base 20..21: mod_base = OTHER; note = thymine
  misc_feature 1..21: note = Synthetic
  source 1..21: mol_type = other RNA
taaactcatgaacatggaatt

SEQ ID NO: 214 - RNA, 21 nt, synthetic construct
  modified_base 20..21: mod_base = OTHER; note = thymine
  misc_feature 1..21: note = Synthetic
  source 1..21: mol_type = other RNA
tatctccaaactcatgaactt

SEQ ID NO: 215 - RNA, 21 nt, synthetic construct
  modified_base 20..21: mod_base = OTHER; note = thymine
  misc_feature 1..21: note = Synthetic
  source 1..21: mol_type = other RNA
ttatctccaaactcatgaatt

SEQ ID NO: 216 - RNA, 21 nt, synthetic construct
  modified_base 20..21: mod_base = OTHER; note = thymine
  misc_feature 1..21: note = Synthetic
  source 1..21: mol_type = other RNA
tgtattatctccaaactcatt

SEQ ID NO: 217 - RNA, 21 nt, synthetic construct
  modified_base 20..21: mod_base = OTHER; note = thymine
  misc_feature 1..21: note = Synthetic
  source 1..21: mol_type = other RNA
ttgtattatctccaaactctt

SEQ ID NO: 218 - RNA, 21 nt, synthetic construct
  modified_base 20..21: mod_base = OTHER; note = thymine
  misc_feature 1..21: note = Synthetic
  source 1..21: mol_type = other RNA
tcctgcactggtacagccttt

SEQ ID NO: 219 - RNA, 21 nt, synthetic construct
  modified_base 20..21: mod_base = OTHER; note = thymine
  misc_feature 1..21: note = Synthetic
  source 1..21: mol_type = other RNA
tacctgcactggtacagcctt

SEQ ID NO: 220 - RNA, 21 nt, synthetic construct
  modified_base 20..21: mod_base = OTHER; note = thymine
  misc_feature 1..21: note = Synthetic
  source 1..21: mol_type = other RNA
tattaaagtgaggacctgctt

SEQ ID NO: 221 - RNA, 21 nt, synthetic construct
  modified_base 20..21: mod_base = OTHER; note = thymine
  misc_feature 1..21: note = Synthetic
  source 1..21: mol_type = other RNA
tgattaaagtgaggacctgtt

SEQ ID NO: 222 - RNA, 21 nt, synthetic construct
  modified_base 20..21: mod_base = OTHER; note = thymine
  misc_feature 1..21: note = Synthetic
  source 1..21: mol_type = other RNA
tgatagaggattaaagtgatt

SEQ ID NO: 223 - RNA, 21 nt, synthetic construct
  modified_base 20..21: mod_base = OTHER; note = thymine
  misc_feature 1..21: note = Synthetic
  source 1..21: mol_type = other RNA
taccgtgttttctggatagtt

SEQ ID NO: 224 - RNA, 21 nt, synthetic construct
  modified_base 20..21: mod_base = OTHER; note = thymine
  misc_feature 1..21: note = Synthetic
  source 1..21: mol_type = other RNA
tcaccgtgttttctggatatt

SEQ ID NO: 225 - RNA, 21 nt, synthetic construct
  modified_base 20..21: mod_base = OTHER; note = thymine
  misc_feature 1..21: note = Synthetic
  source 1..21: mol_type = other RNA
tccaccgtgttttctggattt

SEQ ID NO: 226 - RNA, 21 nt, synthetic construct
  modified_base 20..21: mod_base = OTHER; note = thymine
  misc_feature 1..21: note = Synthetic
  source 1..21: mol_type = other RNA
tgcccaccgtgttttctggtt

SEQ ID NO: 227 - RNA, 21 nt, synthetic construct
  modified_base 20..21: mod_base = OTHER; note = thymine
  misc_feature 1..21: note = Synthetic
  source 1..21: mol_type = other RNA
tttggcccaccgtgttttctt

SEQ ID NO: 228 - RNA, 21 nt, synthetic construct
  modified_base 20..21: mod_base = OTHER; note = thymine
  misc_feature 1..21: note = Synthetic
  source 1..21: mol_type = other RNA
ttttggcccaccgtgtttttt

SEQ ID NO: 229 - RNA, 21 nt, synthetic construct
  modified_base 20..21: mod_base = OTHER; note = thymine
  misc_feature 1..21: note = Synthetic
  source 1..21: mol_type = other RNA
ttcatcctttggcccaccgtt

SEQ ID NO: 230 - RNA, 21 nt, synthetic construct
  modified_base 20..21: mod_base = OTHER; note = thymine
  misc_feature 1..21: note = Synthetic
  source 1..21: mol_type = other RNA
tcatgcctctcttcatccttt

SEQ ID NO: 231 - RNA, 21 nt, synthetic construct
  modified_base 20..21: mod_base = OTHER; note = thymine
  misc_feature 1..21: note = Synthetic
  source 1..21: mol_type = other RNA
tcaacatgcctctcttcattt

SEQ ID NO: 232 - RNA, 21 nt, synthetic construct
  modified_base 20..21: mod_base = OTHER; note = thymine
  misc_feature 1..21: note = Synthetic
  source 1..21: mol_type = other RNA
tgtctccaacatgcctctctt

SEQ ID NO: 233 - RNA, 21 nt, synthetic construct
  modified_base 20..21: mod_base = OTHER; note = thymine
  misc_feature 1..21: note = Synthetic
  source 1..21: mol_type = other RNA
tagtctccaacatgcctcttt

SEQ ID NO: 234 - RNA, 21 nt, synthetic construct
  modified_base 20..21: mod_base = OTHER; note = thymine
  misc_feature 1..21: note = Synthetic
  source 1..21: mol_type = other RNA
ttgcccaagtctccaacattt

SEQ ID NO: 235 - RNA, 21 nt, synthetic construct
  modified_base 20..21: mod_base = OTHER; note = thymine
  misc_feature 1..21: note = Synthetic
  source 1..21: mol_type = other RNA
tattgcccaagtctccaactt

SEQ ID NO: 236 - RNA, 21 nt, synthetic construct
  modified_base 20..21: mod_base = OTHER; note = thymine
  misc_feature 1..21: note = Synthetic
  source 1..21: mol_type = other RNA
tcacattgcccaagtctcctt

SEQ ID NO: 237 - RNA, 21 nt, synthetic construct
  modified_base 20..21: mod_base = OTHER; note = thymine
  misc_feature 1..21: note = Synthetic
  source 1..21: mol_type = other RNA
tgtcagcagtcacattgcctt

SEQ ID NO: 238 - RNA, 21 nt, synthetic construct
  modified_base 20..21: mod_base = OTHER; note = thymine
  misc_feature 1..21: note = Synthetic
  source 1..21: mol_type = other RNA
tttgtcagcagtcacattgtt

SEQ ID NO: 239 - RNA, 21 nt, synthetic construct
  modified_base 20..21: mod_base = OTHER; note = thymine
  misc_feature 1..21: note = Synthetic
  source 1..21: mol_type = other RNA
tccacaccatctttgtcagtt

SEQ ID NO: 240 - RNA, 21 nt, synthetic construct
  modified_base 20..21: mod_base = OTHER; note = thymine
  misc_feature 1..21: note = Synthetic
  source 1..21: mol_type = other RNA
tgccacaccatctttgtcatt

SEQ ID NO: 241 - RNA, 21 nt, synthetic construct
  modified_base 20..21: mod_base = OTHER; note = thymine
  misc_feature 1..21: note = Synthetic
  source 1..21: mol_type = other RNA
tatgcaatggtctcctgagtt

SEQ ID NO: 242 - RNA, 21 nt, synthetic construct
  modified_base 20..21: mod_base = OTHER; note = thymine
  misc_feature 1..21: note = Synthetic
  source 1..21: mol_type = other RNA
tgatgcaatggtctcctgatt

SEQ ID NO: 243 - RNA, 21 nt, synthetic construct
  modified_base 20..21: mod_base = OTHER; note = thymine
  misc_feature 1..21: note = Synthetic
  source 1..21: mol_type = other RNA
tccaatgatgcaatggtcttt

SEQ ID NO: 244 - RNA, 21 nt, synthetic construct
  modified_base 20..21: mod_base = OTHER; note = thymine
  misc_feature 1..21: note = Synthetic
  source 1..21: mol_type = other RNA
tgccaatgatgcaatggtctt

SEQ ID NO: 245 - RNA, 21 nt, synthetic construct
  modified_base 20..21: mod_base = OTHER; note = thymine
  misc_feature 1..21: note = Synthetic
  source 1..21: mol_type = other RNA
ttgcggccaatgatgcaattt

SEQ ID NO: 246 - RNA, 21 nt, synthetic construct
  modified_base 20..21: mod_base = OTHER; note = thymine
  misc_feature 1..21: note = Synthetic
  source 1..21: mol_type = other RNA
taccagtgtgcggccaatgtt

SEQ ID NO: 247 - RNA, 21 nt, synthetic construct
  modified_base 20..21: mod_base = OTHER; note = thymine
  misc_feature 1..21: note = Synthetic
  source 1..21: mol_type = other RNA
tatggaccaccagtgtgcgtt

SEQ ID NO: 248 - RNA, 21 nt, synthetic construct
  modified_base 20..21: mod_base = OTHER; note = thymine
  misc_feature 1..21: note = Synthetic
  source 1..21: mol_type = other RNA
ttcatggaccaccagtgtgtt

SEQ ID NO: 249 - RNA, 21 nt, synthetic construct
  modified_base 20..21: mod_base = OTHER; note = thymine
  misc_feature 1..21: note = Synthetic
  source 1..21: mol_type = other RNA
tttcatggaccaccagtgttt

SEQ ID NO: 250 - RNA, 21 nt, synthetic construct
  modified_base 20..21: mod_base = OTHER; note = thymine
  misc_feature 1..21: note = Synthetic
  source 1..21: mol_type = other RNA
tctttttcatggaccaccatt

SEQ ID NO: 251 - RNA, 21 nt, synthetic construct
  modified_base 20..21: mod_base = OTHER; note = thymine
  misc_feature 1..21: note = Synthetic
  source 1..21: mol_type = other RNA
tctgctttttcatggaccatt

SEQ ID NO: 252 - RNA, 21 nt, synthetic construct
  modified_base 20..21: mod_base = OTHER; note = thymine
  misc_feature 1..21: note = Synthetic
  source 1..21: mol_type = other RNA
tgcccaagtcatctgcttttt

SEQ ID NO: 253 - RNA, 21 nt, synthetic construct
  modified_base 20..21: mod_base = OTHER; note = thymine
  misc_feature 1..21: note = Synthetic
  source 1..21: mol_type = other RNA
ttttgcccaagtcatctgctt

SEQ ID NO: 254 - RNA, 21 nt, synthetic construct
  modified_base 20..21: mod_base = OTHER; note = thymine
  misc_feature 1..21: note = Synthetic
  source 1..21: mol_type = other RNA
tcacctttgcccaagtcattt

SEQ ID NO: 255 - RNA, 21 nt, synthetic construct
  modified_base 20..21: mod_base = OTHER; note = thymine
  misc_feature 1..21: note = Synthetic
  source 1..21: mol_type = other RNA
tccacctttgcccaagtcatt

SEQ ID NO: 256 - RNA, 21 nt, synthetic construct
  modified_base 20..21: mod_base = OTHER; note = thymine
  misc_feature 1..21: note = Synthetic
  source 1..21: mol_type = other RNA
ttccacctttgcccaagtctt

SEQ ID NO: 257 - RNA, 21 nt, synthetic construct
  modified_base 20..21: mod_base = OTHER; note = thymine
  misc_feature 1..21: note = Synthetic
  source 1..21: mol_type = other RNA
tcgtttcctgtctttgtactt

SEQ ID NO: 258 - RNA, 21 nt, synthetic construct
  modified_base 20..21: mod_base = OTHER; note = thymine
  misc_feature 1..21: note = Synthetic
  source 1..21: mol_type = other RNA
tagcgtttcctgtctttgttt

SEQ ID NO: 259 - RNA, 21 nt, synthetic construct
  modified_base 20..21: mod_base = OTHER; note = thymine
  misc_feature 1..21: note = Synthetic
  source 1..21: mol_type = other RNA
tcagcgtttcctgtctttgtt

SEQ ID NO: 260 - RNA, 21 nt, synthetic construct
  modified_base 20..21: mod_base = OTHER; note = thymine
  misc_feature 1..21: note = Synthetic
  source 1..21: mol_type = other RNA
tcgacttccagcgtttccttt

SEQ ID NO: 261 - RNA, 21 nt, synthetic construct
  modified_base 20..21: mod_base = OTHER; note = thymine
  misc_feature 1..21: note = Synthetic
  source 1..21: mol_type = other RNA
tcaccacaagccaaacgactt

SEQ ID NO: 262 - RNA, 21 nt, synthetic construct
  modified_base 20..21: mod_base = OTHER; note = thymine
  misc_feature 1..21: note = Synthetic
  source 1..21: mol_type = other RNA
tacaccacaagccaaacgatt

SEQ ID NO: 263 - RNA, 21 nt, synthetic construct
  modified_base 20..21: mod_base = OTHER; note = thymine
  misc_feature 1..21: note = Synthetic
  source 1..21: mol_type = other RNA
ttacaccacaagccaaacgtt

SEQ ID NO: 264 - RNA, 21 nt, synthetic construct
  modified_base 20..21: mod_base = OTHER; note = thymine
  misc_feature 1..21: note = Synthetic
  source 1..21: mol_type = other RNA
tttacaccacaagccaaactt

SEQ ID NO: 265 - RNA, 21 nt, synthetic construct
  modified_base 20..21: mod_base = OTHER; note = thymine
  misc_feature 1..21: note = Synthetic
  source 1..21: mol_type = other RNA
taattacaccacaagccaatt

SEQ ID NO: 266 - RNA, 21 nt, synthetic construct
  modified_base 20..21: mod_base = OTHER; note = thymine
  misc_feature 1..21: note = Synthetic
  source 1..21: mol_type = other RNA
tccaattacaccacaagcctt

SEQ ID NO: 267 - RNA, 21 nt, synthetic construct
  modified_base 20..21: mod_base = OTHER; note = thymine
  misc_feature 1..21: note = Synthetic
  source 1..21: mol_type = other RNA
tcccaattacaccacaagctt

SEQ ID NO: 268 - RNA, 21 nt, synthetic construct
  modified_base 20..21: mod_base = OTHER; note = thymine
  misc_feature 1..21: note = Synthetic
  source 1..21: mol_type = other RNA
ttcccaattacaccacaagtt

SEQ ID NO: 269 - RNA, 21 nt, synthetic construct
  modified_base 20..21: mod_base = OTHER; note = thymine
  misc_feature 1..21: note = Synthetic
  source 1..21: mol_type = other RNA
tgatcccaattacaccacatt

SEQ ID NO: 270 - RNA, 21 nt, synthetic construct
  modified_base 20..21: mod_base = OTHER; note = thymine
  misc_feature 1..21: note = Synthetic
  source 1..21: mol_type = other RNA
tcgatcccaattacaccactt

SEQ ID NO: 271 - RNA, 21 nt, synthetic construct
  modified_base 20..21: mod_base = OTHER; note = thymine
  misc_feature 1..21: note = Synthetic
  source 1..21: mol_type = other RNA
tgcgatcccaattacaccatt

SEQ ID NO: 272 - RNA, 21 nt, synthetic construct
  modified_base 20..21: mod_base = OTHER; note = thymine
  misc_feature 1..21: note = Synthetic
  source 1..21: mol_type = other RNA
tttgggcgatcccaattactt

SEQ ID NO: 273 - RNA, 21 nt, synthetic construct
  modified_base 20..21: mod_base = OTHER; note = thymine
  misc_feature 1..21: note = Synthetic
  source 1..21: mol_type = other RNA
tattgggcgatcccaattatt

SEQ ID NO: 274 - RNA, 21 nt, synthetic construct
  modified_base 20..21: mod_base = OTHER; note = thymine
  misc_feature 1..21: note = Synthetic
  source 1..21: mol_type = other RNA
ttattgggcgatcccaatttt

SEQ ID NO: 275 - RNA, 21 nt, synthetic construct
  modified_base 20..21: mod_base = OTHER; note = thymine
  misc_feature 1..21: note = Synthetic
  source 1..21: mol_type = other RNA
tttattgggcgatcccaattt

SEQ ID NO: 276 - RNA, 21 nt, synthetic construct
  modified_base 20..21: mod_base = OTHER; note = thymine
  misc_feature 1..21: note = Synthetic
  source 1..21: mol_type = other RNA
ttttattgggcgatcccaatt

SEQ ID NO: 277 - RNA, 21 nt, synthetic construct
  modified_base 20..21: mod_base = OTHER; note = thymine
  misc_feature 1..21: note = Synthetic
  source 1..21: mol_type = other RNA
tgtttattgggcgatcccatt

SEQ ID NO: 278 - RNA, 21 nt, synthetic construct
  modified_base 20..21: mod_base = OTHER; note = thymine
  misc_feature 1..21: note = Synthetic
  source 1..21: mol_type = other RNA
ttgtttattgggcgatccctt

SEQ ID NO: 279 - RNA, 21 nt, synthetic construct
  modified_base 20..21: mod_base = OTHER; note = thymine
  misc_feature 1..21: note = Synthetic
  source 1..21: mol_type = other RNA
tgaatgtttattgggcgattt

SEQ ID NO: 280 - RNA, 21 nt, synthetic construct
  modified_base 20..21: mod_base = OTHER; note = thymine
  misc_feature 1..21: note = Synthetic
  source 1..21: mol_type = other RNA
tcaagggaatgtttattggtt

SEQ ID NO: 281 - RNA, 21 nt, synthetic construct
  modified_base 20..21: mod_base = OTHER; note = thymine
  misc_feature 1..21: note = Synthetic
  source 1..21: mol_type = other RNA
tccaagggaatgtttattgtt

SEQ ID NO: 282 - RNA, 21 nt, synthetic construct
  modified_base 20..21: mod_base = OTHER; note = thymine
  misc_feature 1..21: note = Synthetic
  source 1..21: mol_type = other RNA
ttccaagggaatgtttatttt

SEQ ID NO: 283 - RNA, 21 nt, synthetic construct
  modified_base 20..21: mod_base = OTHER; note = thymine
  misc_feature 1..21: note = Synthetic
  source 1..21: mol_type = other RNA
tatccaagggaatgtttattt

SEQ ID NO: 284 - RNA, 21 nt, synthetic construct
  modified_base 20..21: mod_base = OTHER; note = thymine
  misc_feature 1..21: note = Synthetic
  source 1..21: mol_type = other RNA
tcatccaagggaatgtttatt

SEQ ID NO: 285 - RNA, 21 nt, synthetic construct
  modified_base 20..21: mod_base = OTHER; note = thymine
  misc_feature 1..21: note = Synthetic
  source 1..21: mol_type = other RNA
tacatccaagggaatgttttt

SEQ ID NO: 286 - RNA, 21 nt, synthetic construct
  modified_base 20..21: mod_base = OTHER; note = thymine
  misc_feature 1..21: note = Synthetic
  source 1..21: mol_type = other RNA
ttacatccaagggaatgtttt

SEQ ID NO: 287 - RNA, 21 nt, synthetic construct
  modified_base 20..21: mod_base = OTHER; note = thymine
  misc_feature 1..21: note = Synthetic
  source 1..21: mol_type = other RNA
tgactacatccaagggaattt

SEQ ID NO: 288 - RNA, 21 nt, synthetic construct
  modified_base 20..21: mod_base = OTHER; note = thymine
  misc_feature 1..21: note = Synthetic
  source 1..21: mol_type = other RNA
tcctcagactacatccaagtt

SEQ ID NO: 289 - RNA, 21 nt, synthetic construct
  modified_base 20..21: mod_base = OTHER; note = thymine
  misc_feature 1..21: note = Synthetic
  source 1..21: mol_type = other RNA
ttgagttaaggggcctcagtt

SEQ ID NO: 290 - RNA, 21 nt, synthetic construct
  modified_base 20..21: mod_base = OTHER; note = thymine
  misc_feature 1..21: note = Synthetic
  source 1..21: mol_type = other RNA
tgatgagttaaggggcctctt

SEQ ID NO: 291 - RNA, 21 nt, synthetic construct
  modified_base 20..21: mod_base = OTHER; note = thymine
  misc_feature 1..21: note = Synthetic
  source 1..21: mol_type = other RNA
tagatgagttaaggggccttt

SEQ ID NO: 292 - RNA, 21 nt, synthetic construct
  modified_base 20..21: mod_base = OTHER; note = thymine
  misc_feature 1..21: note = Synthetic
  source 1..21: mol_type = other RNA
taacagatgagttaaggggtt

SEQ ID NO: 293 - RNA, 21 nt, synthetic construct
  modified_base 20..21: mod_base = OTHER; note = thymine
  misc_feature 1..21: note = Synthetic
  source 1..21: mol_type = other RNA
ttaacagatgagttaagggtt

SEQ ID NO: 294 - RNA, 21 nt, synthetic construct
  modified_base 20..21: mod_base = OTHER; note = thymine
  misc_feature 1..21: note = Synthetic
  source 1..21: mol_type = other RNA
tataacagatgagttaaggtt

SEQ ID NO: 295 - RNA, 21 nt, synthetic construct
  modified_base 20..21: mod_base = OTHER; note = thymine
  misc_feature 1..21: note = Synthetic
  source 1..21: mol_type = other RNA
tgataacagatgagttaagtt

SEQ ID NO: 296 - RNA, 21 nt, synthetic construct
  modified_base 20..21: mod_base = OTHER; note = thymine
  misc_feature 1..21: note = Synthetic
  source 1..21: mol_type = other RNA
tggataacagatgagttaatt

SEQ ID NO: 297 - RNA, 21 nt, synthetic construct
  modified_base 20..21: mod_base = OTHER; note = thymine
  misc_feature 1..21: note = Synthetic
  source 1..21: mol_type = other RNA
taggataacagatgagttatt

SEQ ID NO: 298 - RNA, 21 nt, synthetic construct
  modified_base 20..21: mod_base = OTHER; note = thymine
  misc_feature 1..21: note = Synthetic
  source 1..21: mol_type = other RNA
tcaggataacagatgagtttt

SEQ ID NO: 299 - RNA, 21 nt, synthetic construct
  modified_base 20..21: mod_base = OTHER; note = thymine
  misc_feature 1..21: note = Synthetic
  source 1..21: mol_type = other RNA
ttacagctagcaggataactt

SEQ ID NO: 300 - RNA, 21 nt, synthetic construct
  modified_base 20..21: mod_base = OTHER; note = thymine
  misc_feature 1..21: note = Synthetic
  source 1..21: mol_type = other RNA
tcatttctacagctagcagtt

SEQ ID NO: 301 - RNA, 21 nt, synthetic construct
  modified_base 20..21: mod_base = OTHER; note = thymine
  misc_feature 1..21: note = Synthetic
  source 1..21: mol_type = other RNA
tacatttctacagctagcatt

SEQ ID NO: 302 - RNA, 21 nt, synthetic construct
  modified_base 20..21: mod_base = OTHER; note = thymine
  misc_feature 1..21: note = Synthetic
  source 1..21: mol_type = other RNA
taggatacatttctacagctt

SEQ ID NO: 303 - RNA, 21 nt, synthetic construct
  modified_base 20..21: mod_base = OTHER; note = thymine
  misc_feature 1..21: note = Synthetic
  source 1..21: mol_type = other RNA
tcaggatacatttctacagtt

SEQ ID NO: 304 - RNA, 21 nt, synthetic construct
  modified_base 20..21: mod_base = OTHER; note = thymine
  misc_feature 1..21: note = Synthetic
  source 1..21: mol_type = other RNA
ttcaggatacatttctacatt

SEQ ID NO: 305 - RNA, 21 nt, synthetic construct
  modified_base 20..21: mod_base = OTHER; note = thymine
  misc_feature 1..21: note = Synthetic
  source 1..21: mol_type = other RNA
tatcaggatacatttctactt

SEQ ID NO: 306 - RNA, 21 nt, synthetic construct
  modified_base 20..21: mod_base = OTHER; note = thymine
  misc_feature 1..21: note = Synthetic
  source 1..21: mol_type = other RNA
tgtttatcaggatacattttt

SEQ ID NO: 307 - RNA, 21 nt, synthetic construct
  modified_base 20..21: mod_base = OTHER; note = thymine
  misc_feature 1..21: note = Synthetic
  source 1..21: mol_type = other RNA
ttaatgtttatcaggatactt

SEQ ID NO: 308 - RNA, 21 nt, synthetic construct
  modified_base 20..21: mod_base = OTHER; note = thymine
  misc_feature 1..21: note = Synthetic
  source 1..21: mol_type = other RNA
ttaagattacagtgtttaatt

SEQ ID NO: 309 - RNA, 21 nt, synthetic construct
  modified_base 20..21: mod_base = OTHER; note = thymine
  misc_feature 1..21: note = Synthetic
  source 1..21: mol_type = other RNA
tcacttttaagattacagttt

SEQ ID NO: 310 - RNA, 21 nt, synthetic construct
  modified_base 20..21: mod_base = OTHER; note = thymine
  misc_feature 1..21: note = Synthetic
  source 1..21: mol_type = other RNA
tacacttttaagattacagtt

SEQ ID NO: 311 - RNA, 21 nt, synthetic construct
  modified_base 20..21: mod_base = OTHER; note = thymine
  misc_feature 1..21: note = Synthetic
  source 1..21: mol_type = other RNA
ttacacttttaagattacatt

SEQ ID NO: 312 - RNA, 21 nt, synthetic construct
  modified_base 20..21: mod_base = OTHER; note = thymine
  misc_feature 1..21: note = Synthetic
  source 1..21: mol_type = other RNA
tttacacttttaagattactt

SEQ ID NO: 313 - RNA, 21 nt, synthetic construct
  modified_base 20..21: mod_base = OTHER; note = thymine
  misc_feature 1..21: note = Synthetic
  source 1..21: mol_type = other RNA
tcacaattacacttttaagtt

SEQ ID NO: 314 - RNA, 21 nt, synthetic construct
  modified_base 20..21: mod_base = OTHER; note = thymine
  misc_feature 1..21: note = Synthetic
  source 1..21: mol_type = other RNA
tagtttctcactacaggtatt

SEQ ID NO: 315 - RNA, 21 nt, synthetic construct
  modified_base 20..21: mod_base = OTHER; note = thymine
  misc_feature 1..21: note = Synthetic
  source 1..21: mol_type = other RNA
ttcttccaagtgatcataatt

SEQ ID NO: 316 - RNA, 21 nt, synthetic construct
  modified_base 20..21: mod_base = OTHER; note = thymine
  misc_feature 1..21: note = Synthetic
  source 1..21: mol_type = other RNA
taatcttccaagtgatcattt

SEQ ID NO: 317 - RNA, 21 nt, synthetic construct
  modified_base 20..21: mod_base = OTHER; note = thymine
  misc_feature 1..21: note = Synthetic
  source 1..21: mol_type = other RNA
tacaaatcttccaagtgattt

SEQ ID NO: 318 - RNA, 21 nt, synthetic construct
  modified_base 20..21: mod_base = OTHER; note = thymine
  misc_feature 1..21: note = Synthetic
  source 1..21: mol_type = other RNA
taactatacaaatcttccatt

SEQ ID NO: 319 - RNA, 21 nt, synthetic construct
  modified_base 20..21: mod_base = OTHER; note = thymine
  misc_feature 1..21: note = Synthetic
  source 1..21: mol_type = other RNA
tttttaactgagttttatatt

SEQ ID NO: 320 - RNA, 21 nt, synthetic construct
  modified_base 20..21: mod_base = OTHER; note = thymine
  misc_feature 1..21: note = Synthetic
  source 1..21: mol_type = other RNA
tgacattttaactgagttttt

SEQ ID NO: 321 - RNA, 21 nt, synthetic construct
  modified_base 20..21: mod_base = OTHER; note = thymine
  misc_feature 1..21: note = Synthetic
  source 1..21: mol_type = other RNA
tcaggtcattgaaacagactt

SEQ ID NO: 322 - RNA, 21 nt, synthetic construct
  modified_base 20..21: mod_base = OTHER; note = thymine
  misc_feature 1..21: note = Synthetic
  source 1..21: mol_type = other RNA
ttggcaaaatacaggtcattt

SEQ ID NO: 323 - RNA, 21 nt, synthetic construct
  modified_base 20..21: mod_base = OTHER; note = thymine
  misc_feature 1..21: note = Synthetic
  source 1..21: mol_type = other RNA
tgtctggcaaaatacaggttt

SEQ ID NO: 324 - RNA, 21 nt, synthetic construct
  modified_base 20..21: mod_base = OTHER; note = thymine
  misc_feature 1..21: note = Synthetic
  source 1..21: mol_type = other RNA
tagtctggcaaaatacaggtt

SEQ ID NO: 325 - RNA, 21 nt, synthetic construct
  modified_base 20..21: mod_base = OTHER; note = thymine
  misc_feature 1..21: note = Synthetic
  source 1..21: mol_type = other RNA
tatacccatctgtgatttatt

SEQ ID NO: 326 - RNA, 21 nt, synthetic construct
  modified_base 20..21: mod_base = OTHER; note = thymine
  misc_feature 1..21: note = Synthetic
  source 1..21: mol_type = other RNA
tttaatacccatctgtgattt

SEQ ID NO: 327 - RNA, 21 nt, synthetic construct
  modified_base 20..21: mod_base = OTHER; note = thymine
  misc_feature 1..21: note = Synthetic
  source 1..21: mol_type = other RNA
ttttaatacccatctgtgatt

SEQ ID NO: 328 - RNA, 21 nt, synthetic construct
  modified_base 20..21: mod_base = OTHER; note = thymine
  misc_feature 1..21: note = Synthetic
  source 1..21: mol_type = other RNA
tagtttaatacccatctgttt

SEQ ID NO: 329 - RNA, 21 nt, synthetic construct
  modified_base 20..21: mod_base = OTHER; note = thymine
  misc_feature 1..21: note = Synthetic
  source 1..21: mol_type = other RNA
taagtttaatacccatctgtt

SEQ ID NO: 330 - RNA, 21 nt, synthetic construct
  modified_base 20..21: mod_base = OTHER; note = thymine
  misc_feature 1..21: note = Synthetic
  source 1..21: mol_type = other RNA
tcaagtttaatacccatcttt

SEQ ID NO: 331 - RNA, 21 nt, synthetic construct
  modified_base 20..21: mod_base = OTHER; note = thymine
  misc_feature 1..21: note = Synthetic
  source 1..21: mol_type = other RNA
tgacaagtttaatacccattt

SEQ ID NO: 332 - RNA, 21 nt, synthetic construct
  modified_base 20..21: mod_base = OTHER; note = thymine
  misc_feature 1..21: note = Synthetic
  source 1..21: mol_type = other RNA
tgaaattctgacaagtttatt

SEQ ID NO: 333 - RNA, 21 nt, synthetic construct
  modified_base 20..21: mod_base = OTHER; note = thymine
  misc_feature 1..21: note = Synthetic
  source 1..21: mol_type = other RNA
tattcacaggcttgaatgatt

SEQ ID NO: 334 - RNA, 21 nt, synthetic construct
  modified_base 20..21: mod_base = OTHER; note = thymine
  misc_feature 1..21: note = Synthetic
  source 1..21: mol_type = other RNA
ttattcacaggcttgaatgtt

SEQ ID NO: 335 - RNA, 21 nt, synthetic construct
  modified_base 20..21: mod_base = OTHER; note = thymine
  misc_feature 1..21: note = Synthetic
  source 1..21: mol_type = other RNA
tccatacagggtttttatttt

SEQ ID NO: 336 - RNA, 21 nt, synthetic construct
  modified_base 20..21: mod_base = OTHER; note = thymine
  misc_feature 1..21: note = Synthetic
  source 1..21: mol_type = other RNA
tgccatacagggtttttattt

SEQ ID NO: 337 - RNA, 21 nt, synthetic construct
  modified_base 20..21: mod_base = OTHER; note = thymine
  misc_feature 1..21: note = Synthetic
  source 1..21: mol_type = other RNA
ttaagtgccatacagggtttt

SEQ ID NO: 338 - RNA, 21 nt, synthetic construct
  modified_base 20..21: mod_base = OTHER; note = thymine
  misc_feature 1..21: note = Synthetic
  source 1..21: mol_type = other RNA
tataagtgccatacagggttt

SEQ ID NO: 339 - RNA, 21 nt, synthetic construct
  modified_base 20..21: mod_base = OTHER; note = thymine
  misc_feature 1..21: note = Synthetic
  source 1..21: mol_type = other RNA
tgattcttttaatagcctctt

SEQ ID NO: 340 - RNA, 21 nt, synthetic construct
  modified_base 20..21: mod_base = OTHER; note = thymine
  misc_feature 1..21: note = Synthetic
  source 1..21: mol_type = other RNA
ttttgaatttggattcttttt

SEQ ID NO: 341 - RNA, 21 nt, synthetic construct
  misc_feature 1..21: note = Synthetic
  source 1..21: mol_type = other RNA
  modified_base 20..21: mod_base = OTHER; note = thymine
ttagtttgaatttggattctt

SEQ ID NO: 342 - RNA, 21 nt, synthetic construct
  misc_feature 1..21: note = Description of Artificial Sequence: Synthetic oligonucleotide
  source 1..21: mol_type = other RNA
caatgtgactgctgacaaccc

SEQ ID NO: 343 - RNA, 21 nt, synthetic construct
  misc_feature 1..21: note = Description of Artificial Sequence: Synthetic oligonucleotide
  source 1..21: mol_type = other RNA
tttgtcagcagtcacattgtt

SEQ ID NO: 344 - RNA, 21 nt, synthetic construct
  misc_feature 1..21: note = Description of Artificial Sequence: Synthetic oligonucleotide
  source 1..21: mol_type = other RNA
caatgtgactgctgacaatcc

SEQ ID NO: 345 - RNA, 21 nt, synthetic construct
  misc_feature 1..21: note = Description of Artificial Sequence: Synthetic oligonucleotide
  source 1..21: mol_type = other RNA
caatgtgactgctgacaagcc

SEQ ID NO: 346 - RNA, 21 nt, synthetic construct
  misc_feature 1..21: note = Description of Artificial Sequence: Synthetic oligonucleotide
  source 1..21: mol_type = other RNA
caatgtgactgctgacaaacc

SEQ ID NO: 347 - RNA, 21 nt, synthetic construct
  misc_feature 1..21: note = Description of Artificial Sequence: Synthetic oligonucleotide
  source 1..21: mol_type = other RNA
caatgtgacagctgacaaacc

SEQ ID NO: 348 - RNA, 20 nt, synthetic construct
  misc_feature 1..20: note = Description of Artificial Sequence: Synthetic oligonucleotide
  source 1..20: mol_type = other RNA
caatgtgactgctgacaacc

SEQ ID NO: 349 - RNA, 22 nt, synthetic construct
  misc_feature 1..22: note = Description of Artificial Sequence: Synthetic oligonucleotide
  source 1..22: mol_type = other RNA
caatgtgactgctgacaatccc

SEQ ID NO: 350 - RNA, 21 nt, synthetic construct
  misc_feature 1..21: note = Description of Artificial Sequence: Synthetic oligonucleotide
  source 1..21: mol_type = other RNA
caatgtgactgctgacaacac

SEQ ID NO: 351 - RNA, 21 nt, synthetic construct
  misc_feature 1..21: note = Description of Artificial Sequence: Synthetic oligonucleotide
  source 1..21: mol_type = other RNA
tttgtcagcagtcacattgtc

SEQ ID NO: 352 - RNA, 21 nt, synthetic construct
  misc_feature 1..21: note = Description of Artificial Sequence: Synthetic oligonucleotide
  source 1..21: mol_type = other RNA
caatgtgactgctgacaaatc

SEQ ID NO: 353 - RNA, 21 nt, synthetic construct
  misc_feature 1..21: note = Description of Artificial Sequence: Synthetic oligonucleotide
  source 1..21: mol_type = other RNA
caatgtgactgctgacaattc

SEQ ID NO: 354 - RNA, 21 nt, synthetic construct
  misc_feature 1..21: note = Description of Artificial Sequence: Synthetic oligonucleotide
  source 1..21: mol_type = other RNA
tttgtcagcagtcacattgac

SEQ ID NO: 355 - RNA, 22 nt, synthetic construct
  misc_feature 1..22: note = Description of Artificial Sequence: Synthetic oligonucleotide
  source 1..22: mol_type = other RNA
caatgtgactgctgacaatccc

SEQ ID NO: 356 - RNA, 21 nt, synthetic construct
  misc_feature 1..21: note = Description of Artificial Sequence: Synthetic oligonucleotide
  source 1..21: mol_type = other RNA
cgacgaaggccgtgtgcgccc

SEQ ID NO: 357 - RNA, 21 nt, synthetic construct
  misc_feature 1..21: note = Description of Artificial Sequence: Synthetic oligonucleotide
  source 1..21: mol_type = other RNA
tcgcacacggccttcgtcgtt

SEQ ID NO: 358 - RNA, 21 nt, synthetic construct
  misc_feature 1..21: note = Description of Artificial Sequence: Synthetic oligonucleotide
  source 1..21: mol_type = other RNA
tgacttgggcaaaggtggccc

SEQ ID NO: 359 - RNA, 21 nt, synthetic construct
  misc_feature 1..21: note = Description of Artificial Sequence: Synthetic oligonucleotide
  source 1..21: mol_type = other RNA
tccacctttgcccaagtcatt

SEQ ID NO: 360 - RNA, 21 nt, synthetic construct
  misc_feature 1..21: note = Description of Artificial Sequence: Synthetic oligonucleotide
  source 1..21: mol_type = other RNA
aactcatctgttatcctgccc

SEQ ID NO: 361 - RNA, 21 nt, synthetic construct
  misc_feature 1..21: note = Description of Artificial Sequence: Synthetic oligonucleotide
  source 1..21: mol_type = other RNA
tcaggataacagatgagtttt

SEQ ID NO: 362 - RNA, 21 nt, synthetic construct
  misc_feature 1..21: note = Description of Artificial Sequence: Synthetic oligonucleotide
  source 1..21: mol_type = other RNA
ccccttaactcatctgttccc

SEQ ID NO: 363 - RNA, 21 nt, synthetic construct
  misc_feature 1..21: note = Description of Artificial Sequence: Synthetic oligonucleotide
  source 1..21: mol_type = other RNA
taacagatgagttaaggggtt

SEQ ID NO: 364 - RNA, 21 nt, synthetic construct
  misc_feature 1..21: note = Description of Artificial Sequence: Synthetic oligonucleotide
  source 1..21: mol_type = other RNA
cccttaactcatctgttaccc

SEQ ID NO: 365 - RNA, 21 nt, synthetic construct
  misc_feature 1..21: note = Description of Artificial Sequence: Synthetic oligonucleotide
  source 1..21: mol_type = other RNA
ttaacagatgagttaagggtt

SEQ ID NO: 366 - RNA, 21 nt, synthetic construct
  misc_feature 1..21: note = Description of Artificial Sequence: Synthetic oligonucleotide
  source 1..21: mol_type = other RNA
aactcatctgttatcttgccc

SEQ ID NO: 367 - RNA, 21 nt, synthetic construct
  misc_feature 1..21: note = Description of Artificial Sequence: Synthetic oligonucleotide
  source 1..21: mol_type = other RNA
gctgtggaaatgtatcttccc

SEQ ID NO: 368 - RNA, 21 nt, synthetic construct
  misc_feature 1..21: note = Description of Artificial Sequence: Synthetic oligonucleotide
  source 1..21: mol_type = other RNA
taggatacatttctacagctt

SEQ ID NO: 369 - RNA, 21 nt, synthetic construct
  misc_feature 1..21: note = Description of Artificial Sequence: Synthetic oligonucleotide
  source 1..21: mol_type = other RNA
tgacttgggcaaaggtgagcc

SEQ ID NO: 370 - RNA, 21 nt, synthetic construct
  misc_feature 1..21: note = Description of Artificial Sequence: Synthetic oligonucleotide
  source 1..21: mol_type = other RNA
ccccttaactcatctgttgcc

SEQ ID NO: 371 - RNA, 21 nt, synthetic construct
  misc_feature 1..21: note = Description of Artificial Sequence: Synthetic oligonucleotide
  source 1..21: mol_type = other RNA
cccttaactcatctgttagcc

SEQ ID NO: 372 - RNA, 21 nt, synthetic construct
  misc_feature 1..21: note = Description of Artificial Sequence: Synthetic oligonucleotide
  source 1..21: mol_type = other RNA
aactcatctgttatcttagcc

SEQ ID NO: 373 - RNA, 21 nt, synthetic construct
  misc_feature 1..21: note = Description of Artificial Sequence: Synthetic oligonucleotide
  source 1..21: mol_type = other RNA
gctgtggaaatgtatcttgcc

SEQ ID NO: 374 - RNA, 21 nt, synthetic construct
  misc_feature 1..21: note = Description of Artificial Sequence: Synthetic oligonucleotide
  source 1..21: mol_type = other RNA
tgacttgggcaaaggtaggcc

SEQ ID NO: 375 - RNA, 21 nt, synthetic construct
  misc_feature 1..21: note = Description of Artificial Sequence: Synthetic oligonucleotide
  source 1..21: mol_type = other RNA
ccccttaacacatctgttacc

SEQ ID NO: 376 - RNA, 21 nt, synthetic construct
  misc_feature 1..21: note = Description of Artificial Sequence: Synthetic oligonucleotide
  source 1..21: mol_type = other RNA
cccttaactgatctgttaacc

SEQ ID NO: 377 - RNA, 21 nt, synthetic construct
  misc_feature 1..21: note = Description of Artificial Sequence: Synthetic oligonucleotide
  source 1..21: mol_type = other RNA
aactcatctcttatcttgccc

SEQ ID NO: 378 - RNA, 21 nt, synthetic construct
  misc_feature 1..21: note = Description of Artificial Sequence: Synthetic oligonucleotide
  source 1..21: mol_type = other RNA
gctgtggaattgtatcttgcc

SEQ ID NO: 379 - RNA, 21 nt, synthetic construct
  misc_feature 1..21: note = Description of Artificial Sequence: Synthetic oligonucleotide
  source 1..21: mol_type = other RNA
tgacttggggaaaggtgagcc

SEQ ID NO: 380 - RNA, 21 nt, synthetic construct
  misc_feature 1..21: note = Description of Artificial Sequence: Synthetic oligonucleotide
  source 1..21: mol_type = other RNA
aactcatctgttatcttggcc

SEQ ID NO: 381 - RNA, 21 nt, synthetic construct
  misc_feature 1..21: note = Description of Artificial Sequence: Synthetic oligonucleotide
  source 1..21: mol_type = other RNA
ccccttaactcatttgttccc

SEQ ID NO: 382 - RNA, 21 nt, synthetic construct
  misc_feature 1..21: note = Description of Artificial Sequence: Synthetic oligonucleotide
  source 1..21: mol_type = other RNA
tgacttgggcaaaggtagccc

SEQ ID NO: 383 - RNA, 19 nt, synthetic construct
  misc_feature 1..19: note = Description of Artificial Sequence: Synthetic oligonucleotide
  source 1..19: mol_type = other RNA
caatgtgactgctgacaaa

SEQ ID NO: 384 - RNA, 21 nt, synthetic construct
  misc_feature 1..21: note = Description of Artificial Sequence: Synthetic oligonucleotide
  source 1..21: mol_type = other RNA
tttgtcagcagtcacattgtc

SEQ ID NO: 385 - RNA, 21 nt, synthetic construct
  misc_feature 1..21: note = Description of Artificial Sequence: Synthetic oligonucleotide
  source 1..21: mol_type = other RNA
gcaggtcctcactttaatgcc

SEQ ID NO: 386 - RNA, 21 nt, synthetic construct
  misc_feature 1..21: note = Description of Artificial Sequence: Synthetic oligonucleotide
  source 1..21: mol_type = other RNA
gattaaagtgaggacctgctt

SEQ ID NO: 387 - RNA, 21 nt, synthetic construct
  misc_feature 1..21: note = Description of Artificial Sequence: Synthetic oligonucleotide
  source 1..21: mol_type = other RNA
ggcaatgtgactgctgacccc

SEQ ID NO: 388 - RNA, 21 nt, synthetic construct
  misc_feature 1..21: note = Description of Artificial Sequence: Synthetic oligonucleotide
  source 1..21: mol_type = other RNA
tgtcagcagtcacattgcctt

SEQ ID NO: 389 - RNA, 21 nt, synthetic construct
  misc_feature 1..21: note = Description of Artificial Sequence: Synthetic oligonucleotide
  source 1..21: mol_type = other RNA
gcaggtcctcactttaattcc

SEQ ID NO: 390 - RNA, 21 nt, synthetic construct
  misc_feature 1..21: note = Description of Artificial Sequence: Synthetic oligonucleotide
  source 1..21: mol_type = other RNA
ggcaatgtgactgctgatgcc

SEQ ID NO: 391 - RNA, 21 nt, synthetic construct
  misc_feature 1..21: note = Description of Artificial Sequence: Synthetic oligonucleotide
  source 1..21: mol_type = other RNA
gcaggtcctcactttaatccc

SEQ ID NO: 392 - RNA, 21 nt, synthetic construct
  misc_feature 1..21: note = Description of Artificial Sequence: Synthetic oligonucleotide
  source 1..21: mol_type = other RNA
ggcaatgtgactgctgatacc

SEQ ID NO: 393 - RNA, 21 nt, synthetic construct
  misc_feature 1..21: note = Description of Artificial Sequence: Synthetic oligonucleotide
  source 1..21: mol_type = other RNA
gcaggtcctgactttaatccc

SEQ ID NO: 394 - RNA, 21 nt, synthetic construct
  misc_feature 1..21: note = Description of Artificial Sequence: Synthetic oligonucleotide
  source 1..21: mol_type = other RNA
ggcaatgtgtctgctgatacc

SEQ ID NO: 395 - RNA, 22 nt, synthetic construct
  misc_feature 1..22: note = Description of Artificial Sequence: Synthetic oligonucleotide
  source 1..22: mol_type = other RNA
gattaaagtgaggacctgcttt

SEQ ID NO: 396 - RNA, 22 nt, synthetic construct
  misc_feature 1..22: note = Description of Artificial Sequence: Synthetic oligonucleotide
  source 1..22: mol_type = other RNA
tgtcagcagtcacattgccttt